(19) United States
(12) Patent Application Publication
(10) Pub. No.: US 2007/0128593 A1
(43) Pub. Date: Jun. 7, 2007

(54) Sga-1m, a cancer associated antigen, and uses thereof

(75) Inventors: Joseph M. Petroziello, Kenmore, WA (US); Che-Leung Law, Shoreline, WA (US); Alan F. Wahl, Mercer Island, WA (US)

Correspondence Address: SEATTLE GENETICS, INC., 21823 30TH DRIVE SE, BOTHELL, WA, 98021, US

(73) Assignee: SEATTLE GENETICS, INC.

(21) Appl. No.: 10/503,462

(22) PCT Filed: Feb. 3, 2003
(86) PCT No.: PCT/US03/02974

§ 371 Date: Aug. 2, 2004

Related U.S. Application Data
(60) Provisional application No. 60/353,826, filed on Feb. 1, 2002.

(51) Int. Cl. C12Q 1/68

(52) U.S. Cl. 435/6

(57) ABSTRACT

The present invention relates to a gene and gene product, SGA-1M that is differentially expressed in cancer tissues and cell lines. Suppression Subtractive Hybridization and microarray screening were used to screen for differential expression of SGA-1M in cancer tissues and cell lines. Expression analysis has demonstrated overexpression of SGA-1M in breast cancer tissue and breast cancer derived cell lines, in ovarian cancer, skin cancer, a cancer of the lymphoid system, thyroid cancer, pancreatic cancer, stomach cancer, and lung cancer. The gene is expressed as a 1.95 kb mRNA. The full length cDNA comprises two open reading frames encoding polypeptides of 221 and 75 amino acids, respectively. Monitoring expression levels of SGA-1M is useful for the diagnosis and prognosis of cancer as well as for evaluating the risk of developing certain types of cancers and the risk of metastasis of cancer. Reagents that target SGA-1M are useful for the treatment of cancer.

This application claims priority of U.S. Provisional Patent Application No. 60/353,826, filed Feb. 1, 2002, which is incorporated by reference herein in its entirety.

### 1. FIELD OF THE INVENTION

The invention relates generally to the field of cancer diagnosis, prognosis, treatment and prevention. More particularly, the present invention relates to methods of diagnosing, treating and preventing breast cancer, ovarian cancer, skin cancer, cancer of the lymphoid system, thyroid cancer, pancreatic cancer, and stomach cancer. Methods of using a nucleic acid and a protein, differentially expressed in tumor cells, and antibodies against the protein, to treat, diagnose or prevent cancer, are provided for by the present invention. The instant invention provides compositions comprising, and methods of using, products of a gene termed SGA-1M. Such SGA-1M gene products include SGA-1M proteins and nucleic acids. Such gene products, as well as their binding partners and antagonists, can be used for the prevention, diagnosis, prognosis and treatment of cancer.

### 2. BACKGROUND OF THE INVENTION

Cancer is characterized primarily by an increase in the number of abnormal cells derived from a given normal tissue, invasion of adjacent tissues by these abnormal cells, and lymphatic or blood-borne spread of malignant cells to regional lymph nodes and to distant sites (metastasis). Clinical data and molecular biologic studies indicate that cancer is a multistep process that begins with minor preneoplastic changes, which may under certain conditions progress to neoplasia.

Pre-malignant abnormal cell growth is exemplified by hyperplasia, metaplasia, or most particularly, dysplasia (for review of such abnormal growth conditions, see Robbins & Angell, 1976, Basic Pathology, 2d Ed., W. B. Saunders Co., Philadelphia, pp. 68-79) The neoplastic lesion may evolve clonally and develop an increasing capacity for growth, metastasis, and heterogeneity, especially under conditions in which the neoplastic cells escape the host's immune surveillance (Roitt, I., Brostoff, J. and Kale, D., 1993, Immunology, 3rd ed., Mosby, St. Louis, pps. 17.1-17.12).

The incidence of breast cancer, a leading cause of death in women, has been gradually increasing in the United States over the last thirty years. Its cumulative risk is relatively high, 1 in 8 women, for example, by age 85 in the United States. In fact, breast cancer is the most common cancer in women and the second most common cause of cancer death in the United States. In 1997, it was estimated that 181,000 new cases were reported in the U.S., and that 44,000 people would die of breast cancer (Parker et al., 1997, CA Cancer J. Clin. 47:5; Chu et al., 1996, J. Nat. Cancer Inst. 88:1571). While the mechanism of tumorigenesis for most breast carcinomas is largely unknown, there are genetic factors that can predispose some women to developing breast cancer (Miki et al., 1994, Science 266:66). The discovery and characterization of BRCA1 and BRCA2 has expanded our knowledge of genetic factors which can contribute to familial breast cancer. Germ-line mutations within these two loci are associated with a 50 to 85% lifetime risk of breast and/or ovarian cancer (Casey, 1997, Curr. Opin. Oncol. 2:88; Marcus et al., 1996, Cancer 77:697). Sporadic tumors, those not currently associated with a known germline mutation, constitute the majority of breast cancers. It is likely that other, non-genetic factors also have a significant effect on the etiology of the disease. Regardless of its origin, breast cancer morbidity and mortality increases significantly if it is not detected early in its progression. Thus, considerable effort has focused on the early detection of cellular transformation and tumor formation in breast tissue.

Only about 5% to 10% of breast cancers are associated with breast cancer susceptibility genes, BRCA1 and BRCA2. The cumulative lifetime risk of breast cancer for women who carry the mutant BRCA1 is predicted to be approximately 92%, while the cumulative lifetime risk for the non-carrier majority is estimated to be approximately 10%. BRCA1 is a tumor suppressor gene that is involved in DNA repair and cell cycle control, which are both important for the maintenance of genomic stability. More than 90% of all mutations reported so far result in a premature truncation of the protein product with abnormal or abolished function. The histology of breast cancer in BRCA1 mutation carriers differs from that in sporadic cases, but mutation analysis is the only way to find the carrier. Like BRCA1, BRCA2 is involved in the development of breast cancer, and like BRCA1 plays a role in DNA repair. However, unlike BRCA1, it is not involved in ovarian cancer.

Other genes have been linked to breast cancer, for example c-erb-2 (HER2) and p53 (Beenken et al. 2001, Ann. Surg. 233(5):630 ). Overexpression of c-erb-2 (HER2) and p53 have been correlated with poor prognosis (Rudolph et al. 2001, Hum. Pathol. 32(3):311), as has been aberrant expression products of mdm2 (Lukas et al. 2001, Cancer Res. 61(7):3212 ) and cyclin1 and p27 (Porter & Roberts, International Publication WO98/33450, published Aug. 6, 1998).

A marker-based approach to tumor identification and characterization promises improved diagnostic and prognostic reliability. Typically, the diagnosis of breast cancer and other types of cancer requires histopathological proof of the presence of the tumor. In addition to diagnosis, histopathological examinations also provide information about prognosis and selection of treatment regimens. Prognosis may also be established based upon clinical parameters such as tumor size, tumor grade, the age of the patient, and lymph node metastasis.

In clinical practice, accurate diagnosis of various subtypes of cancer is important because treatment options, prognosis, and the likelihood of therapeutic response all vary broadly depending on the diagnosis. Accurate prognosis, or determination of distant metastasis-free survival could allow the oncologist to tailor the administration of adjuvant chemotherapy, with patients having poorer prognoses being given the most aggressive treatment. Furthermore, accurate prediction of poor prognosis would greatly impact clinical trials for new breast cancer therapies, because potential study patients could then be stratified according to prognosis. Trials could then be limited to patients having poor prognosis, in turn making it easier to discern if an experimental therapy is efficacious. To date, no set of satisfactory predictors for prognosis based on the clinical information alone has been identified. The detection of BRCA1 or BRCA2 mutations represents a step towards the design of therapies to better control and prevent the appearance of these tumors.

It would, therefore, be beneficial to provide specific methods and reagents for the diagnosis, staging, prognosis, monitoring and treatment of cancer, including breast cancer, and to provide methods that would identify individuals with a predisposition for the onset of breast cancer, and other types of cancer, and hence are appropriate subjects for preventive therapy.

Intensive and systematic evaluation of gene expression patterns is essential in understanding the physiological mechanisms associated with cellular transformation and metastasis associated with cancer. Several techniques that permit comparison of gene expression in normal and cancerous cells are known in the art. Examples of these techniques include: Serial Analysis of Gene Expression (SAGE) (Velculescu et al., 1995, Science 270:484); Restriction Enzyme Analysis of Differentially Expressed Sequences (READS) (Prasher et al., 1999, Methods in Enzymology 303:258); Amplified Fragment Length Polymorphism (AFLP) (Bachem et al., 1996, Plant Journal 9:745); Representational Difference Analysis (RDA) (Hubank et al., 1994, Nucleic Acid Research 22:(25):5640); differential display (Liang et al., 1992, Cancer Research 52(24):6966); and suppression subtractive hybridization (SSH) (Diatchenko et al., 1996, Proc. Natl. Acad. Sci. USA 93:6025). Such differential expression methods have led the present inventors to the identification and characterization of the SGA-1M gene (see European Patent Application No. EP 1 067 182 A2 and PCT Application No. WO 01/12660) as a gene whose expression is associated with breast cancer and other types of cancer. This discovery by the present inventors has made possible the use of SGA-1M for the treatment, prevention and diagnosis of cancers, including but not limited to breast cancer.

### 3. SUMMARY OF THE INVENTION

The present invention relates to the discovery that a gene, SGA-1M, has an expression pattern that is up-regulated in cancer tissues and cell lines, e.g.,breast cancer tissues and cell lines. The inventors have also discovered that the expression of SGA-1 M is additionally upregulated in other cancers, for example in ovarian, thyroid, melanoma, lymphoma, pancreas, stomach, and lung cancers. Further, the present inventors have identified a novel second open reading frame in the SGA-1M gene. The invention relates to the use of said gene, gene products, and antagonists of said gene or gene products (SGA-1M cDNA, RNA, and/or protein) as targets for diagnosis, drug screening and therapies for cancer. The present invention also relates to the use of said genes or gene products or derivatives thereof as vaccines against cancer. In a preferred embodiment, the invention provides for methods of using the protein, SGA-1M, or nucleic acids which encode said protein for the treatment, prevention and diagnosis of cancer such as breast cancer.

In particular, the methods of the present invention include using nucleic acid molecules that encode the SGA-1M protein, including recombinant DNA molecules, cloned genes or degenerate variants thereof, and in particular naturally occurring variants which encode SGA-1M gene products. The methods of the present invention additionally include using cloning vectors, including expression vectors, containing the nucleic acid molecules encoding SGA-1M and hosts which contain such nucleic acid molecules. The methods of the present invention also encompass the use of SGA-1M gene products, fusion proteins, and antibodies directed against such SGA-1M gene products or conserved variants or fragments thereof. In one embodiment, a fragment or other derivative of an SGA-1M protein is at least 10 amino acids long. In another embodiment, a fragment of an SGA-1M nucleic acid or derivative thereof is at least 10 nucleotides long.

The nucleotide sequence of the cDNA of a human SGA-1M gene (SEQ ID NO:1) is provided. The nucleotide sequences of each of the two ORFs (SEQ ID NO:2 and SEQ ID NO:4) in the SGA-1M gene, as well as the amino acid sequences of their encoded gene products, are also provided (SEQ ID NO:3 and SEQ ID NO:5). As described by way of example in Section 6, the SGA-1M gene was cloned from the human derived breast cancer cell line MCF-7. The SGA-1M gene produces a transcript of approximately 1905 base pairs and encodes proteins of 221 and 75 amino acids. Transcripts were detected at higher levels in several breast cancer cell lines, and in breast tumors as compared to normal tissues. Elevated transcription levels of the SGA-1M gene were also detected in several other tumor types and cancer cells as described in FIG. 14 and in Section 6.

The present invention further relates to methods for the diagnostic evaluation and prognosis of cancer, preferably a carcinoma or adenocarcinoma in a subject animal. Preferably the subject is a mammal, more preferably the subject is a human. In a preferred embodiment the invention relates to methods for diagnostic evaluation and prognosis of breast cancer. For example, nucleic acid molecules of the invention can be used as diagnostic hybridization probes or as primers for diagnostic PCR analysis for detection of abnormal expression of the SGA-1M gene. In other embodiments, the invention relates to methods for diagnostic evaluation and prognosis of ovarian cancer, skin cancer, a cancer of the lymphoid system, thyroid cancer, pancreatic cancer, stomach cancer, or lung cancer.

Antibodies or other binding partners to the SGA-1M protein of the invention can be used in a diagnostic test to detect the presence of the SGA-1M gene product in body fluids, cells or in tissue biopsy. In specific embodiments, measurement of serum or cellular SGA-1M protein levels can be made to detect or stage breast cancer, e.g., infiltrative ductal carcinoma.

The present invention also relates to methods for the identification of subjects having a predisposition to cancer, e.g., breast cancer. The subject can be any animal, but preferably the subject is a mammal, and most preferably the subject is a human. In a non-limiting example nucleic acid molecules of the invention can be used as diagnostic hybridization probes or as primers for quantitative RT-PCR analysis to determine expression levels of the SGA-1M gene product. In another example, nucleic acid molecules of the invention can be used as diagnostic hybridization probes or as primers for diagnostic PCR analysis for the identification of SGA-1M naturally occurring or non-naturally occurring gene mutations, allelic variations and regulatory defects in the SGA-1M gene.

Imaging methods, for imaging the localization and/or amounts of SGA-1M gene products in a patient, are also provided for diagnostic and prognostic use.

Further, methods are presented for the treatment of cancer, including breast cancer. Such methods comprise the administration of compositions that are capable of modulating the level of SGA-1M gene expression and/or the level of SGA-1M gene product activity in a subject. The subject can be any animal, preferably a mammal, more preferably a human.

Still further, the present invention relates to methods for the use of the SGA-I M gene and/or SGA-1M gene products for the identification of compounds which modulate SGA-1M gene expression and/or the activity of SGA-1M gene products. Such compounds can be used as agents to prevent and/or treat breast cancer or any cancer wherein SGA-1M is expressed at levels that are higher than what is found in corresponding normal tissue. Such compounds can also be used to palliate the symptoms of the disease, and control the metastatic potential of breast cancer or any cancer wherein SGA-1M is expressed at levels that are higher than what is found in corresponding normal tissue.

The invention also provides methods of preventing cancer by administering the product of the SGA-1M gene or a fragment of the SGA-1M gene product in an amount effective to elicit an immune response in a subject. The subject can be any animal, preferably a mammal, more preferably a human. The invention also provides methods of treating or preventing cancer by administering the nucleic acid which encodes the SGA-1M gene product or a fragment of the nucleic acid which encodes the SGA-1M gene product in an amount effective to elicit an immune response. The invention further provides methods of treating or preventing cancer by administering a protein or a peptide encoded by the SGA-1M gene in an amount effective to elicit an immune response. The immune response can be either humoral or cellular or both. In a preferred embodiment the invention provides a method of immunizing against breast cancer.

The invention relates to screening assays to identify antagonists or agonists of the SGA-1M gene or gene product. Thus, the invention relates to methods of identifying agonists or antagonists of the SGA-1M gene or gene product and the use of said agonist or antagonist to treat or prevent breast cancer or other types of cancer.

The invention also provides methods of treating cancer by providing therapeutic amounts of an anti-sense nucleic acid molecule. An anti-sense nucleic molecule is a nucleic acid molecule that is the complement of all or a part of the SGA-1M gene sequence (SEQ ID NO:1) or SGA-1M ORFs (SEQ ID NO:2 and SEQ ID NO:4) and which therefore can hybridize to the SGA-1M gene or a fragment thereof. Hybridization of the anti-sense molecule can inhibit expression of the SGA-1M gene. In a preferred embodiment the method is used to treat breast cancer.

The invention also includes a kit for assessing whether a patient is afflicted with breast cancer or other types of cancer. This kit comprises reagents for assessing expression of an SGA-1M gene product.

In another aspect, the invention relates to a kit for assessing the suitability of each of a plurality of compounds for inhibiting cancer including breast cancer in a patient. The kit comprises a reagent for assessing expression of an SGA-1M gene product, and may also comprise a plurality of compounds.

In another aspect, the invention relates to a kit for assessing the presence of cancer cells. This kit comprises an antibody, wherein the antibody binds specifically with a protein corresponding to an SGA-1M gene product. The kit may also comprise a plurality of antibodies, wherein the plurality binds specifically with different epitopes on an SGA-1M gene product.

The invention also includes a kit for assessing the presence of cancer cells, wherein the kit comprises a nucleic acid (e.g.,oligonucleotide) probe. The probe binds specifically with a transcribed polynucleotide corresponding to an SGA-1M gene product. The kit may also comprise a plurality of probes, wherein each of the probes binds specifically with a transcribed polynucleotide corresponding to a different mRNA sequence transcribed from the SGA-1M gene.

Kits for diagnostic use, comprising in a container, primers for use in PCR that can amplify SGA-1M cDNA and/or genes and, in a separate container, a standard amount of SGA-1M cDNA are also provided.

The invention also provides transgenic non-human animals (e.g., mice) which express SGA-1M nucleic acids and proteins encoded by a transgene. Transgenic, non-human knockout animals (e.g., mice), in which an SGA-1M gene has been inactivated, are also provided.

Accordingly, the present invention provides a method of diagnosing cancer in a subject comprising detecting or measuring an SGA-1M gene product in a sample derived from said subject, wherein said SGA-1M gene product is (a) an RNA corresponding to SEQ ID NO:1, or a nucleic acid derived therefrom; (b) a protein comprising SEQ ID NO:3; (c) a protein comprising SEQ ID NO:5; (d) a nucleic acid comprising a sequence hybridizable to SEQ ID NO:1 or its complement under conditions of high stringency, or a protein comprising a sequence encoded by said hybridizable sequence; (e) a nucleic acid comprising a sequence hybridizable to SEQ ID NO:2 or its complement under conditions of high stringency, or a protein comprising a sequence encoded by said hybridizable sequence; (f) a nucleic acid comprising a sequence hybridizable to SEQ ID NO:4 or its complement under conditions of high stringency, or a protein comprising a sequence encoded by said hybridizable sequence; (g) a nucleic acid at least 90% homologous to SEQ ID NO:1 or its complement as determined using the NBLAST algorithm; (h) a nucleic acid at least 90% homologous to SEQ ID NO:2 or its complement as determined using the NBLAST algorithm, or a protein encoded thereby; or (i) a nucleic acid at least 90% homologous to SEQ ID NO:4 or its complement as determined using the NBLAST algorithm, or a protein encoded thereby; in which elevated levels of the SGA-1M gene product compared to a non-cancerous sample or a pre-determined standard value for a noncancerous sample, indicates the presence of cancer in the subject. In one embodiment of the foregoing diagnostic method, the subject is a human. In another embodiment, the cancer is breast cancer. In yet other embodiments, the sample is a tissue sample, a plurality of cells, or a bodily fluid.

The present invention further provides methods of staging cancer in a subject comprising detecting or measuring an SGA-1M gene product in a sample derived from said subject, wherein said SGA-1M gene product is a) an RNA corresponding to SEQ ID NO:1, or a nucleic acid derived therefrom; (b) a protein comprising SEQ ID NO:3; (c) a protein comprising SEQ ID NO:5; (d) a nucleic acid comprising a sequence hybridizable to SEQ ID NO:1 or its complement under conditions of high stringency, or a protein comprising a sequence encoded by said hybridizable sequence; (e) a nucleic acid comprising a sequence hybridizable to SEQ ID NO:2 or its complement under conditions of high stringency, or a protein comprising a sequence encoded by said hybridizable sequence; (f) a nucleic acid comprising a sequence hybridizable to SEQ ID NO:4 or its complement under conditions of high stringency, or a protein comprising a sequence encoded by said hybridizable sequence; (g) a nucleic acid at least 90% homologous to SEQ ID NO:1 or its complement as determined using the NBLAST algorithm; (h) a nucleic acid at least 90% homologous to SEQ ID NO:2 or its complement as determined using the NBLAST algorithm, or a protein encoded thereby; or (i) a nucleic acid at least 90% homologous to SEQ ID NO:4 or its complement as determined using the NBLAST algorithm, or a protein encoded thereby; in which elevated levels of the SGA-1M gene product compared to a non-cancerous sample or a pre-determined standard value for a noncancerous sample, indicates an advanced stage of cancer in the subject.

The present invention further provides methods for the treatment of cancer in a subject, comprising administering to the subject an amount effective for treatment of cancer of a compound that antagonizes an SGA-1M gene product, wherein said SGA-1M gene product is (a) an RNA corresponding to SEQ ID NO:1, or a nucleic acid derived therefrom; (b) a protein comprising SEQ ID NO:3; (c) a protein comprising SEQ ID NO:5; (d) a nucleic acid comprising a sequence hybridizable to SEQ ID NO:1 or its complement under conditions of high stringency, or a protein comprising a sequence encoded by said hybridizable sequence; (e) a nucleic acid comprising a sequence hybridizable to SEQ ID NO:2 or its complement under conditions of high stringency, or a protein comprising a sequence encoded by said hybridizable sequence; (f) a nucleic acid comprising a sequence hybridizable to SEQ ID NO:4 or its complement under conditions of high stringency, or a protein comprising a sequence encoded by said hybridizable sequence; (g) a nucleic acid at least 90% homologous to SEQ ID NO:1 or its complement as determined using the NBLAST algorithm; (h) a nucleic acid at least 90% homologous to SEQ ID NO:2 or its complement as determined using the NBLAST algorithm, or a protein encoded thereby; or (i) a nucleic acid at least 90% homologous to SEQ ID NO:4 or its complemerit as determined using the NBLAST algorithmn, or a protein encoded thereby. In one embodiment, the gene product whose expression is being decreased is a protein encoded by a nucleic acid comprising a nucleotide sequence with at least 90% sequence identity to SEQ ID NO:2. In another embodiment, the gene product whose expression is being decreased is a protein encoded by a nucleic acid comprising a nucleotide sequence with at least 90% sequence identity to SEQ ID NO:4. In other embodiment, the compound decreases expression of an RNA corresponding to SEQ ID NO:1, SEQ ID NO:2, or SEQ ID NO:4. The antagonist can be (i) a protein; (ii) a peptide; (iii) an organic molecule with a molecular weight of less than 500 daltons; (iv) an inorganic molecule with a molecular weight of less than 500 daltons; (v) an antisense oligonucleotide molecule that binds to said RNA and inhibits translation of said RNA; (vi) a ribozyme molecule that targets said RNA and inhibits translation of said RNA; (vii) an antibody that specifically or selectively binds to an SGA-1M gene product; (viii) a double stranded oligonucleotide that forms a triple helix with a promoter of an SGA-1M gene, wherein said SGA-1M gene is a nucleic acid at least 80% homologous to SEQ ID NO:1 or its complement as determined using the NBLAST algorithm; (ix) a double stranded oligonucleotide that forms a triple helix with a promoter of an SGA-1M gene, wherein said SGA-1M gene is a nucleic acid at least 80% homologous to SEQ ID NO:2 or its complement as determined using the NBLAST algorithm; or (x) a double stranded oligonucleotide that forms a triple helix with a promoter of an SGA-1M gene, wherein said SGA-1M gene is a nucleic acid at least 80% homologous to SEQ ID NO:4 or its complement as determined using the NBLAST algorithm. Where the compound is an antibody, in one embodiment, the antibody immunospecifically binds to a protein comprising the amino acid sequence of SEQ ID NO:3; in another, the antibody immunospecifically binds to a protein comprising the amino acid sequence of SEQ ID NO:5.

Th present invention further provides methods of vaccinating a subject against cancer comprising administering to the subject a molecule that elicits an immune response to an SGA-1M gene product, wherein said SGA-1M gene product is (a) an RNA corresponding to SEQ ID NO:1, or a nucleic acid derived therefrom; (b) a protein comprising SEQ ID NO:3; (c) a protein comprising SEQ ID NO:5; (d) a nucleic acid comprising a sequence hybridizable to SEQ ID NO:1 or its complement under conditions of high stringency, or a protein comprising a sequence encoded by said hybridizable sequence; (e) a nucleic acid comprising a sequence hybridizable to SEQ ID NO:2 or its complement under conditions of high stringency, or a protein comprising a sequence encoded by said hybridizable sequence; (f) a nucleic acid comprising a sequence hybridizable to SEQ ID NO:4 or its complement under conditions of high stringency, or a protein comprising a sequence encoded by said hybridizable sequence; (g) a nucleic acid at least 90% homologous to SEQ ID NO:1 or its complement as determined using the NBLAST algorithm; (h) a nucleic acid at least 90% homologous to SEQ ID NO:2 or its complement as determined using the NBLAST algorithm, or a protein encoded thereby; (i) a nucleic acid at least 90% homologous to SEQ ID NO:4 or its complement as determined using the NBLAST algorithm, or a protein encoded thereby; (0) a DNA molecule comprising SEQ ID NO:1; (k) a DNA molecule comprising SEQ ID NO:2; or (l) a DNA molecule comprising SEQ ID NO:4. In one embodiment, the immune response is a cellular immune response. In another embodiment, the immune response is a humoral immune response. In yet another embodiment, the immune response is both a cellular and a humoral immune response.

The present invention yet further provides methods of determining if a subject is at risk of developing cancer, said method comprising (I) measuring an amount of an SGA-1M gene product in a sample derived from the subject, wherein said SGA-1M gene product is: (a) an RNA corresponding to SEQ ID NO:1, or a nucleic acid derived therefrom; (b) a protein comprising SEQ ID NO:3; (c) a protein comprising SEQ ID NO:5; (d) a nucleic acid comprising a sequence hybridizable to SEQ ID NO:1 or its complement under conditions of high stringency, or a protein comprising a sequence encoded by said hybridizable sequence; (e) a nucleic acid comprising a sequence hybridizable to SEQ ID NO:2 or its complement under conditions of high stringency, or a protein comprising a sequence encoded by said hybridizable sequence; (f) a nucleic acid comprising a sequence hybridizable to SEQ ID NO:4 or its complement under conditions of high stringency, or a protein comprising a sequence encoded by said hybridizable sequence; (g) a nucleic acid at least 90% homologous to SEQ ID NO:1 or its complement as determined using the NBLAST algorithm; (h) a nucleic acid at least 90% homologous to SEQ ID NO:2 or its complement as determined using the NBLAST algorithm, or a protein encoded thereby; or (i) a nucleic acid at least 90% homologous to SEQ ID NO:4 or its complement as determined using the NBLAST algorithm, or a protein encoded thereby; and (II) comparing the amount of said SGA-1M gene product in the subject with the amount of SGA-1M gene product present in a non-cancerous sample or predetermined standard for a noncancerous sample, wherein an elevated amount of said SGA-1M gene product in the subject compared to the amount in the non-cancerous sample or predetermined standard for a noncancerous sample indicates a risk of developing cancer in the subject.

The present invention yet further provides methods of determining if a subject suffering from cancer is at risk of metastasis of said cancer, said method comprising measuring an amount of an SGA-1M gene product in a sample derived from the subject, wherein said gene product is (a) an RNA corresponding to SEQ ID NO:1, or a nucleic acid derived therefrom; (b) a protein comprising SEQ ID NO:3; (c) a protein comprising SEQ ID NO:5; (d) a nucleic acid comprising a sequence hybridizable to SEQ ID NO:1 or its complement under conditions of high stringency, or a protein comprising a sequence encoded by said hybridizable sequence; (e) a nucleic acid comprising a sequence hybridizable to SEQ ID NO:2 or its complement under conditions of high stringency, or a protein comprising a sequence encoded by said hybridizable sequence; (f) a nucleic acid comprising a sequence hybridizable to SEQ ID NO:4 or its complement under conditions of high stringency, or a protein comprising a sequence encoded by said hybridizable sequence; (g) a nucleic acid at least 90% homologous to SEQ ID NO:1 or its complement as determined using the NBLAST algorithm; (h) a nucleic acid at least 90% homologous to SEQ ID NO:2 or its complement as determined using the NBLAST algorithm, or a protein encoded thereby; or (i) a nucleic acid at least 90% homologous to SEQ ID NO:4 or its complement as determined using the NBLAST algorithm, or a protein encoded thereby; wherein an elevated amount of SGA-1M gene product in the subject compared to the amount in the non-cancerous sample, or in the sample from the subject with the non-metastasizing cancer, or the amount in the predetermined standard for a noncancerous or non-metastasizing sample, indicates a risk of developing metastasis of said cancer in the subject.

The present invention yet further provides methods of screening for a compound that binds with an SGA-1M molecule, said method comprising (I) contacting the SGA-1M molecule with a candidate agent, wherein said SGA-1M molecule is (a) an RNA corresponding to SEQ ID NO: 1, or a nucleic acid derived therefrom; (b) a protein comprising SEQ ID NO:3; (c) a protein comprising SEQ ID NO:5; (d) a nucleic acid comprising a sequence hybridizable to SEQ ID NO:1 or its complement under conditions of high stringency, or a protein comprising a sequence encoded by said hybridizable sequence; (e) a nucleic acid comprising a sequence hybridizable to SEQ ID NO:2 or its complement under conditions of high stringency, or a protein comprising a sequence encoded by said hybridizable sequence; (f) a nucleic acid comprising a sequence hybridizable to SEQ ID NO:4 or its complement under conditions of high stringency, or a protein comprising a sequence encoded by said hybridizable sequence; (g) a nucleic acid at least 90% homologous to SEQ ID NO:1 or its complement as determined using the NBLAST algorithm; (h) a nucleic acid at least 90% homologous to SEQ ID NO:2 or its complement as determined using the NBLAST algorithm, or a protein encoded thereby; or (i) a nucleic acid at least 90% homologous to SEQ ID NO:4 or its complement as determined using the NBLAST algorithm, or a protein encoded thereby; and (II) determining whether or not the candidate agent binds the SGA-1M molecule. The screening assay can be performed in vitro. In one embodiment, the SGA-1M molecule is anchored to a solid phase. In another embodiment, the candidate agent is anchored to a solid phase. In other embodiments, the screening assay is performed in the liquid phase. In yet other embodiments, the SGA-1M molecule is expressed on the surface of a cell or in the cytosol of a cell in step (I). In the latter embodiments, the SGA-1M molecule can be naturally expressed by the cell; alternatively, the cell can be engineered to express the SGA-1M molecule. In the foregoing screening methods, the candidate agent is preferably labeled, for example radioactively or enzymatically.

The present invention provides methods of screening for an intracellular protein that interacts with an SGA-1M gene product, said method comprising (I) immunoprecipitating the SGA-1M gene product from a cell lyrate, wherein said SGA-1M gene product is (a) an RNA corresponding to SEQ ID NO:1, or a nucleic acid derived therefrom; (b) a protein comprising SEQ ID NO:3; (c) a protein comprising SEQ ID NO:5; (d) a nucleic acid comprising a sequence hybridizable to SEQ ID NO:1 or its complement under conditions of high stringency, or a protein comprising a sequence encoded by said hybridizable sequence; (e) a nucleic acid comprising a sequence hybridizable to SEQ ID NO:2 or its complement under conditions of high stringency, or a protein comprising a sequence encoded by said hybridizable sequence; (f) a nucleic acid comprising a sequence hybridizable to SEQ ID NO:4 or its complement under conditions of high stringency, or a protein comprising a sequence encoded by said hybridizable sequence; (g) a nucleic acid at least 90% homologous to SEQ ID NO:1 or its complement as determined using the NBLAST algorithm; (h) a nucleic acid at least 90% homologous to SEQ ID NO:2 or its complement as determined using the NBLAST algorithm, or a protein encoded thereby; or (i) a nucleic acid at least 90% homologous to SEQ ID NO:4 or its complement as determined using the NBLAST algorithm, or a protein encoded thereby; and (II) determining whether or not any intracellular proteins bind to or form a complex with the SGA-1M gene product in the immunoprecipitate.

The present invention yet further provides methods of screening for a candidate agent that modulates expression level of an SGA-1M gene, said method comprising (I) contacting said SGA-1M gene with a candidate agent, wherein said SGA-1M gene is a nucleic acid at least 80% homologous to SEQ ID NO:1 as determined using the NBLAST algorithm; and (II) measuring the level of expression of an SGA-1M gene product, said SGA-1M gene product selected from the group consisting of an mRNA corresponding to SEQ ID NO:1, a protein comprising SEQ ID NO:3, and a protein comprising SEQ ID NO:5, wherein an increase or decrease in said level of expression relative to said level of expression in the absence of said candidate agent indicates that the candidate agent modulates expression of an SGA-1M gene.

The present invention yet further provides methods of screening for a compound that is a candidate cancer therapeutic agent. In certain embodiment, such a method of screening comprises: (a) contacting an SOA-1M polypeptide, either in vitro or in vivo (e.g., by contacting comprising cell that expresses the SGA-1M polypeptide) with a compound and (b) determining whether an SGA-1M activity is modulated (i.e., increased, inhibited or altered) by the compound, thereby identifying a candidate cancer therapeutic agent. A compound that modulates an SGA-1M activity is a candidate cancer therapeutic agent. In one embodiment, the SGA-1M polypeptide is an SGA-1M(A) polypeptide. In another embodiment, the SGA-1M polypeptide is an SGA-1M(B) polypeptide. In certain embodiments, the activity modulated is (a) a subcellular localization of the SGA-1M polypeptide, (b) an interaction between the SGA-1M polypeptide (e.g., a SGA-1M(A) polypeptide) and a binding partner (e.g., a Nedd-4 protein), (c) a post-translational modification of the SGA-1M polypeptide (e.g., ubiquitination of SGA-1M(A)), or (d) an activity of a protein (e.g., a sodium channel) whose activity is regulated or modulated by the SGA-1M polypeptide (e.g., a SGA-1M(A) polypeptide).

The present invention yet further provides a vaccine formulation for the prevention of cancer comprising (I) an immunogenic amount of an SGA-1M gene product, wherein said SGA-1M gene product is: (a) an RNA corresponding to SEQ ID NO:1, or a nucleic acid derived therefrom; (b) a protein comprising SEQ ID NO:3; (c) a protein comprising SEQ ID NO:5; (d) a nucleic acid comprising a sequence hybridizable to SEQ ID NO:1 or its complement under conditions of high stringency, or a protein comprising a sequence encoded by said hybridizable sequence; (e) a nucleic acid comprising a sequence hybridizable to SEQ ID NO:2 or its complement under conditions of high stringency, or a protein comprising a sequence encoded by said hybridizable sequence; (f) a nucleic acid comprising a sequence hybridizable to SEQ ID NO:4 or its complement under conditions of high stringency, or a protein comprising a sequence encoded by said hybridizable sequence; (g) a nucleic acid at least 90% homologous to SEQ ID NO: 1 or its complement as determined using the NBLAST algorithm; (h) a nucleic acid at least 90% homologous to SEQ ID NO:2 or its complement as determined using the NBLAST algorithm, or a protein encoded thereby; or (i) a nucleic acid at least 90% homologous to SEQ ID NO:4 or its complement as determined using the NBLAST algorithm, or a protein encoded thereby; and (II) a pharmaceutically acceptable excipient.

The present invention yet further provides and immunogenic composition comprising (I) a purified SGA-1M gene product in an amount effective at eliciting an immune response, wherein said gene product is (a) an RNA corresponding to SEQ ID NO:1, or a nucleic acid derived therefrom; (b) a protein comprising SEQ ID NO:3; (c) a protein comprising SEQ ID NO:5; (d) a nucleic acid comprising a sequence hybridizable to SEQ ID NO:1 or its complement under conditions of high stringency, or a protein comprising a sequence encoded by said hybridizable sequence; (e) a nucleic acid comprising a sequence hybridizable to SEQ ID NO:2 or its complement under conditions of high stringency, or a protein comprising a sequence encoded by said hybridizable sequence; (f) a nucleic acid comprising a sequence hybridizable to SEQ ID NO:4 or its complement under conditions of high stringency, or a protein comprising a sequence encoded by said hybridizable sequence; (g) a nucleic acid at least 90% homologous to SEQ ID NO: 1 or its complement as determined using the NBLAST algorithm; (h) a nucleic acid at least 90% homologous to SEQ ID NO:2 or its complement as determined using the NBLAST algorithm, or a protein encoded thereby; or (i) a nucleic acid at least 90% homologous to SEQ ID NO:4 or its complement as determined using the NBLAST algorithm, or a protein encoded thereby; and (II) an excipient.

The present invention yet further provides a pharmaceutical composition comprising an antibody which specifically or selectively binds to a protein consisting essentially of SEQ ID NO:3; and a pharmaceutically acceptable carrier. The present invention yet further provides a pharmaceutical composition comprising an antibody which specifically or selectively binds to a protein consisting essentially of SEQ ID NO:5; and a pharmaceutically acceptable carrier.

The present invention yet further provides pharmaceutical compositions comprising (I) an SGA-1M gene product, wherein said gene product is The present invention yet further provides a pharmaceutical composition comprising an antibody which specifically or selectively binds to a protein comprising SEQ ID NO:3; and a pharmaceutically acceptable carrier; and (II) a pharmaceutically acceptable carrier.

The present invention yet further provides a pharmaceutical composition comprising (I) a purified nucleic acid comprising SEQ ID NO:2 or SEQ ID NO:4; and (II) a pharmaceutically acceptable carrier.

The pharmaceutical compositions of the present invention can be formulated, inter alia, for delivery as an aerosol, for parenteral delivery, or for oral delivery.

The present invention yet further provides methods of diagnosing cancer in a subject comprising (I) administering to said subject a compound that specifically binds a protein consisting essentially of the amino acid sequence of SEQ ID NO:3, wherein said compound is bound to an imaging agent; and (II) obtaining an internal image of said subject by use of said imaging agent; wherein the localization or amount of said image indicates whether or not cancer is present in said subject. The present invention yet further provides methods of diagnosing cancer in a subject comprising (I) administering to said subject a compound that specifically binds a protein consisting essentially of the amino acid sequence of SEQ ID NO:5, wherein said compound is bound to an imaging agent; and (II) obtaining an internal image of said subject by use of said imaging agent; wherein the localization or amount of said image indicates whether or not cancer is present in said subject. In a preferred embodiment, the compound is an antibody. In a preferred mode of the embodiment, the antibody is conjugated to a radioactive metal and said obtaining step comprises recording a scintographic image obtained from the decay of the radioactive metal.

The present invention yet further provides kits that are useful for practicing the present methods. In one embodiment, such a kit comprises, in one or more containers, a pair of oligonucleotide primers, each primer comprising a nucleotide sequence with at least 5 complementary nucleotides to a different strand of a double-stranded nucleic acid comprising SEQ ID NO:1; and, in a separate container, a purified double-stranded nucleic acid comprising SEQ ID NO:1. In specific modes of the embodiment, each primer comprises a nucleotide sequence with at least 8, more preferably at least 10, yet more preferably at least 12, and most preferably at least 15 complementary nucleotides to a different strand of a double-stranded nucleic acid comprising SEQ ID NO:1.

The present invention yet further provides transgenic non-human animal which express from a transgene an SGA-1M gene product, for example, an RNA corresponding to SEQ ID NO:1, a protein comprising SEQ ID NO:3 or a protein comprising SEQ ID NO:5.

The present invention yet further provides a method of testing the effects of a candidate therapeutic compound comprising administering said compound to a transgenic non-human animal which express from a transgene an SGA-1M gene product; and determining any effects of said compound upon said transgenic non-human animal.

The present invention provides an isolated polypeptide comprising at least 8, at least 10, at least 15, at least 20 or at least 50 amino acids of SEQ ID NO:5. least 10 amino acids of SEQ ID NO:5. In one embodiment, the polypeptide is purified.

The present invention further provides an isolated polypeptide which is encoded by a nucleic acid molecule comprising a nucleotide sequence which is at least 90% identical to a nucleic acid consisting of the nucleotide sequence of any of SEQ ID NO:4.

The present invention further provides host cells comprising nucleic acids encoding the polypeptides of the invention operably linked to a promoter, and methods of expressing such polypeptides by culturing the host cells under conditions in which the nucleic acid molecule is expressed.

### 3.1 Definitions

SPECIFIC: a nucleic acid used in a reaction, such as a probe used in a hybridization reaction, a primer used in a PCR, or a nucleic acid present in a pharmaceutical preparation, is referred to as “specific” if it hybridizes or reacts only with the intended target. Similarly, a polypeptide is referred to as “specific” if it binds only to its intended target, such as a ligand, hapten, substrate, antibody, or other polypeptide. An antibody is referred to as “specific” if it binds only to the intended target. A marker is specific to a particular cell or tissue type if it is detectably expressed only in or on that cell or tissue type.

SELECTIVE: a nucleic acid used in a reaction, such as a probe used in a hybridization reaction, a primer used in a PCR, or a nucleic acid present in a pharmaceutical preparation, is referred to as “selective” if it hybridizes or reacts with the intended target more frequently, more rapidly, or with greater duration than it does with alternative substances. Similarly, a polypeptide is referred to as “selective” if it binds an intended target, such as a ligand, hapten, substrate, antibody, or other polypeptide more frequently, more rapidly, or with greater duration than it does to alternative substances. An antibody is referred to as “selective” if it binds via at least one antigen recognition site to the intended target more frequently, more rapidly, or with greater duration than it does to alternative substances. A marker is selective to a particular cell or tissue type if it is expressed predominantly in or on that cell or tissue type, particularly with respect to a biological sample of interest.

CORRESPOND OR CORRESPONDING: Between nucleic acids, “corresponding” means homologous to or complementary to a particular sequence or portion of the sequence of a nucleic acid. As between nucleic acids and polypeptides, “corresponding” refers to amino acids of a peptide in an order derived from the sequence or portion of the sequence of a nucleic acid or its complement. As between polypeptides (or peptides and polypeptides), “corresponding” refers to amino acids of a first polypeptide (or peptide) in an order derived from the sequence or portion of the sequence of a second polypeptide.

SGA-1M GENE PRODUCT: As used herein, unless otherwise indicated, the term “an SGA-1M gene product” includes, but it not limited, to the following molecules: an RNA corresponding to SEQ ID NO:1, or a nucleic acid derived therefrom; a protein comprising SEQ ID NO:3; a protein comprising SEQ ID NO:5; a nucleic acid comprising a sequence hybridizable to SEQ ID NO:1 or its complement under conditions of high stringency, or a protein comprising a sequence encoded by said hybridizable sequence; a nucleic acid comprising a sequence hybridizable to SEQ ID NO:2 or its complement under conditions of high stringency, or a protein comprising a sequence encoded by said hybridizable sequence; a nucleic acid comprising a sequence hybridizable to SEQ ID NO:4 or its complement under conditions of high stringency, or a protein comprising a sequence encoded by said hybridizable sequence; a nucleic acid at least 90% homologous to SEQ ID NO:1 or its complement as determined using the NBLAST algorithm; a nucleic acid at least 90% homologous to SEQ ID NO:2 or its complement as determined using the NBLAST algorithm, or a protein encoded thereby; a nucleic acid at least 90% homologous to SEQ ID NO:4 or its complement as determined using the NBLAST algorithm, or a protein encoded thereby; or a fragment or derivative of any of the foregoing proteins or nucleic acids, including a fragment or derivative that is capable of immunospecifically binding to an anti-SGA-1M antibody or encodes a protein that is capable of immunospecifically binding to an anti-SGA-1M antibody. Other SGA-1M gene products are described in Sections 5.1 and 5.2 below.

### 4. BRIEF DESCRIPTION OF THE FIGURES

FIG. 1. Breast-specific expression arrays of Suppression Subtractive Hybridization (SSH) clones probed with cDNA isolated from the breast cancer cell-line, MCF-7 (A) and normal Human Mammary Epithelial Cells (HMEC) (B). Tumor-selective genes Cytokeratin 8, and Cytokeratin 18 along with the control gene EF-1 are included for comparison.

FIG. 2. The SGA-1M transcript. The entire cDNA sequence of 1905 bp with coding sequences (CDS) spanning 173-835 bp (+2 frame) (A), and 1104-1328 bp (+3 frame) (B). The putative start ATG is located at position 173-175 bp (+2 frame) (A), and 1104-1106 bp (+3 frame) (B).

FIG. 3. Normal and transformed breast cells are evaluated for SGA-1M expression by Northern analysis. SGA-1M cDNA from 225-706 bp was amplified and used as probe. One ug of poly A+ RNA was loaded in each lane. Samples are as follows: (1) MCF-7, (2) normal Human Mammary Epithelial Cells, (3) SKBR-3, (4) MDA-MB-231, (5) MDA-MB-435s, (6) Hs578T, and (7) BT549. The control gene EF-1 was included for comparison.

FIG. 4. Semi-quantitative RT-PCR of normal vs. transformed breast cells. One hundred ng of poly A+ RNA was used to synthesize cDNA for this experiment. SGA-1M cDNA from 272-482 bp was amplified in this assay. RT-PCR products were visualized by ethidium bromide staining. Samples are loaded as follows: (1) MCF-7, (2) normal Human Mammary Epithelial Cells, (3) SKBR-3, (4) MDA-MB-231, (5) MDA-MB435s, (6) Hs578T, and (7) BT549. The control gene EF-1 was included for comparison.

FIG. 5. Semi-quantitative RT-PCR for SGA-1M on various ATCC tumor cell-lines. Five ug of total RNA was used to synthesize cDNA for this experiment. SGA-1M cDNA spanning 272-482 bp was amplified. RT-PCR products were separated on 1.2% agarose gels and visualized by ethidium bromide staining. Samples are loaded as follows: (1) T47D, (2) MCF-7, (3) SKBR-3, (4) MDA-MB231, (5) MDA-MB435s, (6) Hs578T, (7) BT549, (8) L2987, (9) WM2664, (10) NIH:OVCAR3, (11) SK-OV3, (12) PA-1, (13) Daudi, (14) Raji, and (15) Ramos. The control gene EF-1 was included for comparison.

FIG. 6. Tissue type and location of various poly A+ RNA's and controls found on the Multiple Tissue Expression Array (MTE), as illustrated in FIG. 7.

FIG. 7. Analysis of EF-1 control gene (A), and SGA-1M (B) transcript expression levels on the Multiple Tissue Expression Array, as detailed in FIG. 6. SGA-1M cDNA spanning 225-706 bp was amplified and used as a probe. The control gene EF-1 was included for comparison.

FIG. 8. Tissue type and location of 241 tumor/normal sample pairs isolated from individual patients as spotted on the Cancer Profiling Array (CPA), as illustrated in FIG. 9. Numbers across the top of the grid from left to right (1-48) represent patient isolate pairs. Letters (A-FF) are included as line designations for ease of data analysis. The distribution of the 241 patient isolates, includes: Breast (50), Uterus (42), Colon (35), Stomach (27), Ovary (14), Cervix (1), Lung (21), Kidney (20), Rectum (18), Small Intestine (2), Thyroid (6), Prostate (4), and Pancreas (1).

FIG. 9. SGA-1M transcript expression analysis on 241 patient isolates using the Cancer Profiling Array (CPA), as detailed in FIG. 8. Tumor/normal pairs with SGA-1M tumor-selective expression are indicated by arrows.

FIG. 10. Amino acid sequence for SGA-1M proteins spanning 221 aa (+2 open reading frame) (A), and 75 aa (+3 open reading frame)(B), as detailed in FIG. 2. Hydrophobic regions are indicated in bold and labeled with a TM designation. Hydrophilic regions corresponding to synthesized peptide used to raise polyclonal antibodies are noted as SGA-1M (1-4) and SGA-1M (1-2).

FIG. 11. Specificity of the anti-SGA-1M antibodies. SGA-1M/Myc-His fusion protein constructs were used to determine the specificity of anti-SGA-1M (1-2) and anti-SGA-1M (1-4). Detergent lysates were prepared from COS-7 cells transiently expressing SGA-1M/Myc-His. Anti-SGA-1M(1-2), anti-SGA-1M(1-4), and anti-Myc were used to immunoprecipitate SGA-1M/Myc-His from the cell lysates. The presence of endogenous SGA-1M and SGA-1M/Myc-His was detected by immunoblotting with either anti-Myc or anti-SGA-1M(1-4). Rabbit IgG and mouse IgG were included as negative controls for immunoprecipitation.

FIG. 12. Breast tissue Immunohistochemistry (IHC) staining using the established murine antibody for the tumor-selective marker BR96. Images include, staining with rabbit IgG alone (A), staining with BR96 on normal breast tissue (B), and staining with BR96 on primary breast tumor tissue (C).

FIG. 13. Breast tissue Immunohistochemistry (IHC) staining using anti-SGA-1M (1-2) and anti-SGA-1M (1-4) polyclonal antibodies, as outlined in FIG. 10. Images include staining with anti-SGA-1M (1-4) on normal breast tissue (A), breast tumor tissue (B), and peptide, (1-4) blocking on breast tumor tissue (C). In addition, staining with anti-SGA-1M (1-2) on normal breast tissue (D), breast tumor tissue (E), and peptide, (1-2) blocking on breast tumor tissue (F).

FIG. 14. IHC staining using the anti-SGA-1M (1-4) polyclonal antibody, as outlined in FIG. 10, on multiple carcinoma types. Images include positive staining with anti-SGA-1M (1-4) on breast adenocarcinoma (A), melanoma (B), thyroid carcinoma (C), lymphoma (D), pancreas adenocarcinoma (E), and stomach adenocarcinoma (F).

FIG. 15. Subcellular localization of SGA-1M. Subcellular localization was determined by analyzing the expression of an SGA-1M/green fluorescence protein (GFP) fusion construct. GFP alone (A) and (B), or SGA-1M/GFP (C) and (D) were transiently expressed in COS-7 or Vero cells. The localization of green fluorescence signals was determined by fluorescence microscopy.

FIG. 16. Alignments of SGA-1M with related GenBank nucleic acid sequences.

FIG. 17. Alignments of SGA-1M with related GenBank amino acid sequences.

### 5. DETAILED DESCRIPTION OF THE INVENTION

The present invention relates to the discovery that the SGA-1M gene is over-expressed in cancer cells and tissues such as breast cancer cells. The invention relates to methods of using the SGA-1M gene and/or the SGA-1M gene products to diagnose, treat and prevent cancer, e.g., breast cancer. The invention further relates to methods of using the SGA-1M gene or SGA-1M gene products to evaluate the prognosis of a patient diagnosed with cancer. The invention also relates to the discovery that the SGA-1M gene is over-expressed in metastatic cancer cells. Thus, the invention contemplates the use of the SGA-1M gene and/or gene products to evaluate a cancer patient's risk of the metastasis of said cancer, e.g.,breast cancer.

In the development of breast neoplasia and other cancers, there are a subset of genes that will be specifically expressed at various stages, and a certain number of these will be critical for the progression of malignancy, especially those associated with the metastatic spread of the disease. As described by way of example, infra, genes whose expression is associated with breast carcinomas in various stages of neoplastic development, were identified using Suppression Subtractive Hybridization (SSH) and high-throughput cDNA microarray (Chu et al., 1997, Proc. Natl. Acad. Sci. U.S.A. 94(19):10057; Kuang et al., 1998, Nuc. Acids Res. 26(4):1116 ). SSH generated cDNA libraries derived from the breast cancer cell line MCF-7 were screened using microarrays for genes which were expressed at elevated levels in the cancerous MCF-7 cells as compared to normal breast cells. A total of 576 clones were screened. Several previously identified breast cancer associated genes, as well as the SGA-1M gene were identified by this analysis. The details concerning the isolation and characterization of the full length SGA-1M clone and its association with cancer cell lines and tissues is described in detail in the examples provided infra.

The present invention encompasses methods for the diagnosis, prognosis and staging of breast cancer and other cancers, e.g., by the monitoring of the effect of a therapeutic treatment. Further provided are methods for the use of the SGA-1M gene and/or SGA-1M gene products in the identification of compounds which modulate the expression of the SGA-1M gene or the activity of the SGA-1M gene product. Expression of the SGA-1M gene is upregulated in various types of cancer cells including breast cancer cell lines and tissues. As such, the SGA-1M gene product can be involved in the mechanisms underlying the onset and development of breast cancer and other types of cancer as well as the regional infiltration and metastatic spread of cancer. Thus, the present invention also provides methods for the prevention and/or treatment of breast cancer and other types of cancer, and for the control of metastatic spread of breast cancer and other types of cancer that is based on modulation of the expression of the SGA-1M gene or gene product.

The invention further provides for screening assays and methods of identifying agonists and antagonists of the SGA-1M gene or gene product. The invention also provides methods of vaccinating an individual against cancer, including breast cancer, by administering an amount of the SGA-1M gene, gene product, or fragment thereof, in an amount which effectively elicits an immune response in a subject who has cancer or is at risk of developing cancer, including breast cancer.

### 5.1 The SGA-1M Gene

Nucleotide sequences which encode the SGA-1M gene open reading frame are described herein. The full-length SGA-1M cDNA (1905 bp) (SEQ ID NO:1) was cloned from a MCF-7 cDNA library. The DNA sequence contains two open reading frames (SEQ ID NO:2 and SEQ ID NO:4) that encode proteins of 221 and 75 amino acids, referred to herein as SGA-1M(A)(SEQ ID NO:3) and SGA-1M(B) (SEQ ID NO:5), respectively. The amino acid sequence of SGA-1M(A) shows 80% homology to the mouse homolog of Nedd 4.

The SGA-1M nucleic acids and derivatives used in the present invention include but are not limited to RNA corresponding to SEQ ID NO:1 or a nucleic acid derived therefrom, including but not limited to RNAs comprising SEQ ID NO:2 and/or SEQ ID NO:4; a nucleic acid comprising a sequence hybridizable to SEQ ID NO:1, SEQ ID NO:2, SEQ ID NO:4, or the complement of any of the foregoing nucleic acids; a nucleic acid at least 90% homologous to SEQ ID NO:1, SEQ ID NO:2, SEQ ID NO:4, or at least 90% homologous the complement of any of the foregoing nucleic acids (e.g., as determined using the NBLAST algorithm under default parameters). As used herein an “RNA corresponding to SEQ ID NO:1/2/4” means an RNA comprising a sequence that is the same or the (inverse) complement of SEQ ID NO:1/2/4, except that thymidines (T's) can be replaced with uridines (U's). Such RNAs corresponding to SEQ ID NO:1 include for example RNA encoded by a gene that gives rise to a cDNA of SEQ ID NO:1, as well as RNA of which the cDNA of SEQ ID NO:1 is a copy. A nucleic acid derived from such an RNA includes but is not limited to cDNA of said RNA, and cRNA (e.g., RNA that is derived from said cDNA; see, e.g., U.S. Pat. Nos. 5,545,522; 5,891,636; 5,716,785). In the present invention, hybridizability can be determined under low, moderate, or high stringency conditions and preferably is under conditions of high stringency.

The SGA-1M proteins and derivatives used in the present invention include, but are not limited to proteins (and other molecules) comprising SEQ ID NO:3, SEQ ID NO:5, proteins comprising a sequence encoded by the hybridizable (complementary) portion of a nucleic acid hybridizable to SEQ ID NO:2 or SEQ ID NO:4 or their complements, and proteins encoded by a nucleic acid at least 90% homologous to SEQ ID NO:1, SEQ ID NO:2, SEQ ID NO:4 or their complement, e.g., as determined using the NBLAST algorithm.

The SGA-1M nucleic acids used in the present invention include but are not limited to (a) a DNA comprising the DNA sequence shown in FIG. 2 (SEQ ID NO:1) or its complement; (b) any DNA sequence that hybridizes to the DNA sequences or their complements that encode the amino acid sequences shown in FIG. 2, under low, moderate or highly stringent conditions, as disclosed infra in Section 5.1.1; as well as proteins encoded by such nucleic acids. In a specific embodiment, nucleic acids used in the invention encode a gene product that has at least one conservative or silent substitution. The encoded proteins are also provided for use. Additional molecules that can be used in the invention include, but are not limited to, protein derivatives that can be made by altering their sequences by substitutions, additions or deletions, and their encoding nucleic acids. Due to the degeneracy of nucleotide coding sequences, other DNA sequences that encode substantially the same amino acid sequence as a component gene or cDNA can be used in the practice of the present invention. These include but are not limited to nucleotide sequences comprising all or portions of the component protein gene that are altered by the substitution of different codons that encode a functionally equivalent amino acid residue within the sequence, thus producing a silent change. Likewise, the derivatives of the invention include, but are not limited to, those containing, as a primary amino acid sequence, all or part of the amino acid sequence of a component protein, including altered sequences in which functionally equivalent amino acid residues are substituted for residues within the sequence resulting in a silent change. For example, one or more amino acid residues within the sequence can be substituted by another amino acid of a similar polarity (a “conservative amino acid substitution”) that acts as a functional equivalent, resulting in a silent alteration. Substitutes for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid.

The invention includes the use of the SGA-1M gene coding sequences which preferably hybridize under highly stringent or moderately stringent conditions as described infra in Section 5.1.1 to at least about 6, preferably about 12, more preferably about 18, consecutive nucleotides of the SGA-1M gene sequences described above as being useful for the detection of an SGA-1M gene product for the diagnosis and prognosis of cancer, e.g., an RNA corresponding to SEQ ID NO:1, or a nucleic acid derived therefrom; a nucleic acid comprising a sequence hybridizable to SEQ ID NO:1 or its complement under conditions of high stringency; a nucleic acid comprising a sequence hybridizable to SEQ ID NO:2 or its complement under conditions of high stringency; a nucleic acid comprising a sequence hybridizable to SEQ ID NO:4 or its complement under conditions of high stringency; a nucleic acid at least 90% homologous to SEQ ID NO:1 or its complement as determined using the NBLAST algorithm; a nucleic acid at least 90% homologous to SEQ ID NO:2 or its complement; or a nucleic acid at least 90% homologous to SEQ ID NO:4 or its complement.

The invention also includes the use of nucleic acid molecules, preferably DNA molecules, that preferably hybridize under highly stringent or moderately stringent conditions as described infra in Section 5.1.1 to, and are therefore the inverse complements of, the nucleic acid sequences (a) and (d)-(i), described, inter alia, in Section 3 above. These nucleic acid molecules may encode or act as SGA-1M gene coding sequence antisense molecules useful, for example, in SGA-1M gene regulation. With respect to SGA-1M gene regulation, such techniques can be used to modulate, for example, the phenotype and metastatic potential of breast cancer or other cancer cells. Further, such sequences may be used as part of ribozyme and/or triple helix sequences, also useful for SGA-1M gene regulation and thus may be used for the treatment and/or prevention of cancer.

In one embodiment, the invention encompasses methods of using the SGA-1M gene coding sequence or fragments and degenerate variants of DNA sequences which encode the SGA-1M gene or gene product, including naturally occurring and non-naturally occurring variants thereof. A non-naturally occurring variant is one that is engineered by man. A naturally occurring SGA gene, gene product, or variant thereof is one that is not engineered by man. In the methods of the invention wherein an SGA-1M gene product in a sample derived from a subject is detected or measured, naturally occurring SGA-1M gene products are detected, including, but not limited to wild-type SGA-1M gene products as well as mutants, allelic variants, splice variants, polymorphic variants, etc. In general, such mutants and variants are believed to be highly homologous to SEQ ID NO:1, SEQ ID NO:2 or SEQ ID NO:4, e.g., at least 90% homologous and/or hybridizable under high stringency conditions. In specific embodiments, the mutants and variants being detected or measured comprise ( or, if nucleic acids, encode) not more than 1, 2, 3, 4, or 5 point mutations (substitutions) relative to SEQ ID NO:1, SEQ ID NO:2 or SEQ ID NO:4, and or comprise or encode only conservative amino acid substitutions.

In other methods of the invention, wild-type, or naturally occurring variant, or non-naturally occurring variant SGA-1M sequences may be used in the methods of the invention (e.g., in vaccination, immunization, antisense, or ribozyme procedures).

An SGA-1M gene fragment may be a complementary DNA (cDNA) molecule or a genomic DNA molecule that may comprise one or more intervening sequences or introns, as well as regulating regions located beyond the 5′ and 3′ ends of the coding region or within an intron.

The present invention provides for methods of using isolated nucleic acid molecules encoding an SGA-1M protein, polypeptide, or fragments, derivatives, and variants thereof which include, both naturally occurring and non-naturally occurring variants or mutants. The invention also contemplates, for use in the methods of the invention, the use of 1) any nucleic acid that encodes an SGA-1M polypeptide of the invention; 2) any nucleic acid that hybridizes to the complement of the sequences disclosed herein, preferably under highly stringent conditions as disclosed infra in Section 5.1.1, and encodes a functionally equivalent gene product; and/or 3) any nucleic acid sequence that hybridizes to the complement of the sequences disclosed herein, preferably under moderately stringent conditions, as disclosed infra in Section 5.1.1 yet which still encodes a gene product that displays a functional activity of SGA-1M.

As discussed above, the invention also contemplates the use of isolated nucleic acid molecules that encode a variant protein or polypeptide. The variant protein or polypeptide can occur naturally or non-naturally. It can be engineered by introducing nucleotide substitutions, e.g., point mutations, or additions or deletions into the nucleotide sequence of SEQ ID NO:1, SEQ ID NO:2 or SEQ ID NO:4. In a specific embodiment, one or more, but not more than 5, 10, or 25 amino acid substitutions, additions or deletions are introduced into the encoded protein. Mutations can be introduced by standard techniques, such as site-directed mutagenesis and PCR-mediated mutagenesis. Preferably, conservative amino acid substitutions are made at one or more predicted non-essential amino acid residues. Following mutagenesis, the encoded protein can be expressed recombinantly and the activity of the protein can be determined.

In a specific embodiment, the invention provides for the use of SGA-1M derivatives and analogs of the invention which are functionally active, i.e., they are capable of displaying one or more known functional activities associated with a full-length (wild-type) SGA-1M-encoded protein. Such functional activities include but are not limited to antigenicity (ability to bind (or compete with SGA-1M(A) or SGA-1M(B) for binding) to an anti-SGA-1M(A) or anti-SGA-1(B) antibody, respectively), immunogenicity (ability to generate antibody which binds to SGA-1M(A) or SGA-1(B)), ability to bind (or compete with SGA-1M(A) or SGA-1(B) for binding) to other proteins or fragments thereof, ability to bind (or compete with SGA-1M(A) or SGA-1(B) for binding) to a receptor for SGA-1M.

Using all or a portion of the nucleic acid sequences of SEQ ID NO:1, for example SEQ ID NO:2, SEQ ID NO:4 or portions thereof, as a hybridization probe, nucleic acid molecules encoding an SGA-1M gene product can be isolated using standard hybridization and cloning techniques (See, e.g., Sambrook et al., Molecular Cloning: A Laboratory Manual, 2nd ed., Cold Spring Harbor Laboratory, Cold Spring Harbor Laboratory Press, Cold Spring Harbor, N.Y., 1989) for use in the methods of the invention.

In addition, gene products encoded by SGA-1M, including SGA-1M(A) or SGA-1M(B) peptide fragments, as well as specific or selective antibodies thereto, can be used for construction of fusion proteins to facilitate recovery, detection, or localization of another protein of interest. In addition, genes and gene products encoded for by SGA-1M (e.g., SGA-1M(A) or SGA-1M(B)) can be used as a research reagent, e.g., for genetic mapping.

Additionally, the present invention contemplates use of the nucleic acid molecules, polypeptides, and/or antagonists of gene products encoded for by the SGA-1M gene to screen, diagnose, prevent and/or treat disorders characterized by aberrant expression or activity of the SGA-1M(A) or SGA-1M(B) polypeptides, which include, cancers, such as but not limited to cancer of the breast, ovary, skin and lymphoid system.

The present invention encompasses the use of SGA-1M nucleic acid molecules comprising cDNA, genomic DNA, introns, exons, promoter regions, 5′ and 3′ regulatory regions of the gene, RNA, hnRNA, mRNA, regulatory regions within RNAs, and degenerate variants thereof in the methods of the invention. Promoter sequences for SGA-1M can be determined by promoter-reporter gene assays and in vitro binding assays.

In one embodiment, the invention comprises the use of a variant SGA-1M nucleic acid sequence that hybridizes to a naturally-occurring or non-naturally occurring variant SGA-1M nucleic acid molecule under stringent conditions as described infra in Section 5.1.1. In another embodiment, the invention contemplates the use of an SGA-1M variant nucleic acid sequence that hybridizes to a naturally-occurring or non-naturally occurring variant SGA-1M nucleic acid molecule under moderately stringent conditions as described infra in Section 5.1.1.

A nucleic acid molecule is intended to include DNA molecules (e.g., cDNA, genomic DNA), RNA molecules (e.g., hnRNA, pre-mRNA, mRNA), and DNA or RNA analogs generated using nucleotide analogs. The nucleic acid molecule can be single-stranded or double-stranded.

The SGA-1M gene sequences used in the methods of the invention are of human origin, however, homologs of SGA-1M isolated from other mammals may also be used in the methods of the invention. Thus, the invention also includes the use of SGA-1M homologs isolated from non-human animals such as non-human primates; rats; mice; farm animals including, but not limited to: cattle; horses; goats; sheep; pigs; etc.; household pets including, but not limited to: cats; dogs; etc. in the methods of the invention.

Still further, such molecules may be used as components of diagnostic and/or prognostic methods whereby, for example, the presence of a particular SGA-1M allele or alternatively spliced SGA-1M transcript responsible for causing or predisposing one to breast cancer or other cancers may be detected.

The invention also includes the use of transcriptional regulators which control the level of expression of an SGA-1M gene product. A transcriptional regulator can include, e.g., a protein which binds a DNA sequence and which up-regulates or down regulates the transcription of the SGA-1M gene. A transcriptional regulator can also include a nucleic acid sequence which can be either up stream or down stream from the SGA-1M gene and which binds an effector molecule that enhances or suppresses SGA-1M gene transcription.

Still further, the invention encompasses the use of SGA-1M gene coding sequences or fragments thereof as a screen in an engineered yeast system, including, but not limited to, the yeast two hybrid system as a method to identify proteins, peptides or nucleic acids related to the onset and or metastatic spread of cancer, including breast cancer.

The invention also encompasses the use of (a) DNA vectors that contain any of the foregoing SGA-1M coding sequences and/or their complements (e.g., antisense); (b) DNA expression vectors that contain any of the foregoing SGA-1M coding sequences operatively associated with a regulatory element that directs the expression of the coding sequences; and (c) genetically engineered host cells that contain any of the foregoing SGA-1M coding sequences operatively associated with a regulatory element that directs the expression of the coding sequences in the host cell. Cell lines and/or vectors which contain and/or express SGA-1M can be used to produce the SGA-1M gene product for use in the methods of the invention, e.g., vaccination against breast cancer or other cancers in which expression of SGA-1M is found to be elevated and screening assays for antagonists and agonists that bind, or interact with SGA-1M or suppress or enhance expression of SGA-1M.

As used herein, regulatory elements include, but are not limited to inducible and non-inducible promoters, enhancers, operators and other elements known to those skilled in the art that drive and regulate expression. Such regulatory elements include but are not limited to the cytomegalovirus (hCMV) immediate early promoter, the early or late promoters of SV40 adenovirus, the lac system, the trp system, the TAC system, the TRC system, the major operator and promoter regions of phage A, the control regions of fd coat protein, the promoter for 3-phosphoglycerate kinase, the promoters of acid phosphatase, and the promoters of the yeast α-mating factors.

The invention includes the use of fragments or derivatives of any of the nucleic acids disclosed herein in any of the methods of the invention. In various embodiments, a fragment or derivative comprises 10, 20, 50, 100, or 200 nucleotides of SEQ ID NO:1, SEQ ID NO:2 or SEQ ID NO:4, or encodes all or a fragment of SEQ ID NO:3 or SEQ ID NO:5. In the same or alternative embodiments, a nucleic acid is not more than 500, 1000, 2000, 5000, 7500, 10,000, 15,000, 20,000, 50,000 or 100,000 nucleotides in size.

In addition to the use of the SGA-1M gene sequences described above, homologs of such sequences, exhibiting extensive homology to the SGA-1M gene product present in other species can be identified and readily isolated, and used in the methods of the invention without undue experimentation, by molecular biological techniques well known in the art. Further, there can exist homolog genes at other genetic loci within the genome that encode proteins which have extensive homology to SGA-1M(A) and/or SGA-1(B). Such homologous genes, like SGA-1M, can encode two proteins, one or both of which are homologous to SGA-1M(A) and/or SGA-1(B). Alternatively, such homologous genes can encode a single protein with homology to SGA-1M(A) or SGA-1M(B). These genes can also be identified via similar techniques and used in the methods of the invention. Still further, there can exist alternatively spliced variants of the SGA-1M gene. The invention thus includes the use of any of these homologs in the methods of the invention.

As an example, in order to clone a mammalian SGA-1M gene homolog or variants using isolated human SGA-1M gene sequences as disclosed herein, such human SGA-1M gene sequences are labeled and used to screen a cDNA library constructed from mRNA obtained from appropriate cells or tissues (e.g., breast epithelial cells) derived from the organism of interest. With respect to the cloning of such a mammalian SGA-1M homolog, a mammalian breast cancer cell cDNA library may, for example, be used for screening. In one embodiment, such a screen would employ a probe corresponding to all or a portion of the SGA-1M(A) open reading frame (SEQ ID NO:2). In another embodiment, such a screen would employ a probe corresponding to all or a portion of the SGA-1M(B) open reading frame (SEQ ID NO:4). In yet another embodiments, such a screen would employ one or more probes corresponding to all or a portion of each of the SGA-1M(A) and SGA-1M(B) open reading frames, for example, a probe corresponding to the SGA-1M cDNA (SEQ ID NO:1).

The hybridization and wash conditions used should be of a low stringency, as described infra in Section 5.1.1 when the cDNA library is derived from a different type of organism than the one from which the labeled sequence was derived.

Alternatively, the labeled fragment may be used to screen a genomic library derived from the organism of interest, again, using appropriately stringent conditions well known to those of skill in the art.

Further, an SGA-1M gene homolog may be isolated from nucleic acid of the organism of interest by performing PCR using two degenerate oligonucleotide primer pools designed on the basis of amino acid sequences within an SGA-1M encoded gene product, for example by performing PCR using two degenerate oligonucleotide primer pools corresponding to portions of either SGA-1M(A) or SGA-1M(B). The template for the reaction may be cDNA obtained by reverse transcription of mRNA prepared from, for example, mammalian cell lines or tissue known or suspected to express an SGA-1M gene homology or allele.

The PCR product may be subcloned and sequenced to ensure that the amplified sequences represent the sequences of an SGA-1M-related nucleic acid sequence. The PCR fragment may then be used to isolate a full length cDNA clone by a variety of methods. For example, the amplified fragment may be labeled and used to screen a cDNA library, such as a bacteriophage cDNA library. Alternatively, the labeled fragment may be used to isolate genomic clones via the screening of a genomic library.

PCR technology may be utilized to isolate full length cDNA sequences. For example, RNA may be isolated, following standard procedures, from an appropriate cellular or tissue source (e.g., one known, or suspected, to express the SGA-1M gene, such as, for example, breast cancer cell lines). A reverse transcription reaction may be performed on the RNA using an oligonucleotide primer specific or selective for the most 5′ end of the amplified fragment for the priming of first strand synthesis. The resulting RNA/DNA hybrid may then be “tailed” with guanines using a standard terminal transferase reaction, the hybrid may be digested with RNAase H, and second strand synthesis may then be primed with a poly-C primer. Thus, cDNA sequences upstream of the amplified fragment may easily be isolated. For a review of PCR technology and cloning strategies which may be used, see, e.g., PCR Primer, 1995, Dieffenbach et al., ed., Cold Spring Harbor Laboratory Press; Sambrook et al., 1989, supra.

SGA-1M gene coding sequences may additionally be used to isolate SGA-1M gene alleles and mutant SGA-1M gene alleles. Such mutant alleles may be isolated from individuals either known or susceptible to or predisposed to have a genotype which contributes to the development of cancer, e.g., breast cancer, including metastasis. Such mutant alleles may also be isolated from individuals either known or susceptible to or predisposed to have a genotype which contributes to resistance to the development of cancer, e.g., breast cancer, including metastasis. Mutant alleles and mutant allele products may then be utilized in the screening, therapeutic and diagnostic methods and systems described herein. Additionally, such SGA-1M gene sequences can be used to detect SGA-1M gene regulatory (e.g., promoter) defects which can affect the development and outcome of cancer. Mutants can be isolated by any technique known in the art, e.g., PCR, screening genomic libraries, screening expression libraries.

As described below, the invention also relates to the use of an SGA-1M gene coding sequence or gene product in the methods of the invention. An SGA-1M gene coding sequence or gene product includes, but is not limited to an RNA corresponding to SEQ ID NO:1, SEQ ID NO:2 or SEQ ID NO:4, a nucleic acid derived therefrom, a protein comprising SEQ ID NO:3 or SEQ ID NO:5, a nucleic acid comprising a sequence hybridizable to SEQ ID NO:1, SEQ ID NO:2 or SEQ ID NO:4 under conditions of high stringency, or a protein comprising a sequence encoded by said hybridizable sequence or a nucleic acid at least 90% homologous to SEQ ID NO:1, SEQ ID NO:2 or SEQ ID NO:4 as determined by the NBLAST algorithm or a protein encoded thereby.

### 5.1.1 Hybrdization Conditions

A nucleic acid which is hybridizable to an SGA-1M nucleic acid (e.g., having a sequence as set forth in SEQ ID NO:1, SEQ ID NO:2 or SEQ ID NO:4), or to its reverse complement, or to a nucleic acid encoding an SGA-1M derivative, or to its reverse complement under conditions of low stringency can be used in the methods of the invention to detect the presence of an SGA-1M gene and/or presence or expression level of an SGA-1M gene product. By way of example and not limitation, procedures using such conditions of low stringency are as follows (see also Shilo and Weinberg, 1981, Proc. Natl. Acad. Sci. U.S.A. 78, 6789-6792). Filters containing DNA are pretreated for 6 h at 40° C. in a solution containing 35% formamide, 5×SSC, 50 mM Tris-HCl (pH 7.5), 5 mM EDTA, 0.1% PVP, 0.1% Ficoll, 1% BSA, and 500 μg/ml denatured salmon spermn DNA. Hybridizations are carried out in the same solution with the following modifications: 0.02% PVP, 0.02% Ficoll, 0.2% BSA, 100 μg/ml salmon sperm DNA, 10% (wt/vol) dextran sulfate, and 5-20×10⁶ cpm ³²P-labeled probe is used. Filters are incubated in hybridization mixture for 18-20 h at 40° C., and then washed for 1.5 h at 55° C. in a solution containing 2×SSC, 25 mM Tris-HCl (pH 7.4), 5 mM EDTA, and 0.1% SDS. The wash solution is replaced with fresh solution and incubated an additional 1.5 h at 60° C. Filters are blotted dry and exposed for autoradiography. If necessary, filters are washed for a third time at 65-68° C. and re-exposed to film. Other conditions of low stringency which may be used are well known in the art (e.g., as employed for cross-species hybridizations).

A nucleic acid which is hybridizable to an SGA-1M nucleic acid (e.g., having a sequence as set forth in SEQ ID NO:1, SEQ ID NO:2, or SEQ ID NO:4), or to its reverse complement, or to a nucleic acid encoding an SGA-1M derivative, or to its reverse complement under conditions of high stringency is also provided for use in the methods of the invention. By way of example and not limitation, procedures using such conditions of high stringency are as follows. Prehybridization of filters containing DNA is carried out for 8 h to overnight at 65° C. in buffer composed of 6×SSC, 50 mM Tris-HCl (pH 7.5), 1 mM EDTA, 0.02% PVP, 0.02% Ficoll, 0.02% BSA, and 500 μg/ml denatured salmon sperm DNA. Filters are hybridized for 48 h at 65° C. in prehybridization mixture containing 100 μg/ml denatured salmon sperm DNA and 5-20×10⁶ cpm of ³²P-labeled probe. Washing of filters is done at 37° C. for 1 h in a solution containing 2×SSC, 0.01% PVP, 0.01% Ficoll, and 0.01% BSA. This is followed by a wash in 0.1×SSC at 50° C. for 45 min before autoradiography. Other conditions of high stringency which may be used are well known in the art.

A nucleic acid which is hybridizable to an SGA-1M nucleic acid (e.g., having a sequence as set forth in SEQ ID NO:1, SEQ ID NO:2, or SEQ ID NO:4), or to its reverse complement, or to a nucleic acid encoding an SGA-1M derivative, or to its reverse complement under conditions of moderate stringency is also provided for use in the methods of the invention. For example, but not limited to, procedures using such conditions of moderate stringency are as follows: Filters containing DNA are pretreated for 6 hours at 55° C. in a solution containing 6×SSC, 5× Denhardt's solution, 0.5% SDS and 100 μg/ml denatured salmon sperm DNA. Hybridizations are carried out in the same solution with 5-20×10⁶ cpm ³²P-labeled probe. Filters are incubated in hybridization mixture for 18-20 hours at 55° C., and then washed twice for 30 minutes at 60° C. in a solution containing 1×SSC and 0.1% SDS. Filters are blotted dry and exposed for autoradiography. Washing of filters is done at 37° C. for 1 hour in a solution containing 2×SSC, 0.1% SDS. Other conditions of moderate stringency which may be used are well-known in the art. (see, e.g., Sambrook et al., 1989, Molecular Cloning, A Laboratory Manual, 2d Ed., Cold Spring Harbor Laboratory Press, Cold Spring Harbor, N.Y.; see also, Ausubel et al., eds., in the Current Protocols in Molecular Biology series of laboratory technique manuals, 1987-1997 Current Protocols,© 1994-1997 John Wiley and Sons, Inc.).

As used herein, the term “nucleic acid”, when referring to an SGA-1M nucleic acid, does not encompass (a) a genome or (b) a nucleic acid molecule when in a library, such as a genomic or cDNA library.

### 5.2. Protein Products of the SGA-1M Gene

In another embodiment, the present invention provides for the use of SGA-1M gene products, including SGA-1M(A), SGA-1M(B), or peptide fragments thereof which can be used for the generation of antibodies, in diagnostic assays, or for the identification of other cellular gene products involved in the development of cancer, such as, for example, breast cancer.

The amino acid sequences depicted in FIGS. 2A and 2B represent examples of SGA-1M gene products, i.e., SGA-1M(A) and SGA-1M(B) (SEQ ID NO:3 and SEQ ID NO:5, respectively). The SGA-1M gene products, sometimes referred to herein as an “SGA-1M proteins” or “SGA-1M polypeptides,” may additionally include those gene products encoded by the SGA-1M gene sequences described in Section 5.1, above.

In addition, SGA-1M derivatives may include proteins that have conservative amino acid substitution(s) and/or display a functional activity of an SGA-1M gene product, including but not limited to SGA-1M(A) and SGA-1M(B). Such a derivative may contain deletions, additions or substitutions of amino acid residues within the amino acid sequence encoded by the SGA-1M gene sequences described, above, in Section 5.1, but which result in a silent change, thus producing a functionally equivalent SGA-1M gene product.

In a specific embodiment, the invention provides a functionally equivalent protein that exhibits a substantially similar in vivo activity as an endogenous SGA-1M gene product encoded by an SGA-1M gene sequence described in Section 5.1, above. An in vivo activity of the SGA-1M gene product can be exhibited by, for example, preneoplastic and/or neoplastic transformation of a cell upon overexpression of the gene product, such as for example, may occur in the onset and progression and metastasis of breast cancer.

An SGA-1M gene product sequence preferably comprises an amino acid sequence that exhibits at least about 65% sequence similarity to SGA-1M(A) or SGA-1M(B), more preferably exhibits at least 70% sequence similarity to SGA-1M(A) or SGA-1M(B), yet more preferably exhibits at least about 75% sequence similarity to SGA-1M(A) or SGA-1M(B). In other embodiments, the SGA-1M gene product sequence preferably comprises an amino acid sequence that exhibits at least 85% sequence similarity to SGA-1M(A) or SGA-1M(B), yet more preferably exhibits at least 90% sequence similarity to to SGA-1M(A) or SGA-1M(B), and most preferably exhibits at least about 95% sequence similarity to SGA-1M(A) or SGA-1M(B).

In other embodiments of the present invention, an SGA-1M gene product sequence preferably-comprises an amino acid sequence that exhibits at least about 65% sequence identity to SGA-1M(A) or SGA-1M(B), more preferably exhibits at least 70% sequence identity to SGA-1M(A) or SGA-1M(B), yet more preferably exhibits at least about 75% sequence identity to SGA-1M(A) or SGA-1M(B). In yet other embodiments, the SGA-1M gene product sequence preferably comprises an amino acid sequence that exhibits at least 85% sequence identity to SGA-1M(A) or SGA-1M(B), yet more preferably exhibits at least 90% sequence identity to to SGA-1M(A) or SGA-1M(B), and most preferably exhibits at least about 95% sequence identity to SGA-1M(A) or SGA-1M(B).

The determination of percent identity between two sequences can be accomplished using a mathematical algorithm. A preferred, non-limiting example of a mathematical algorithm utilized for the comparison of two sequences is the algorithm of Karlin and Altschul (1990) Proc Natl Acad Sci. 87:2264-2268, modified as in Karlin and Altschul (1993) Proc Natl Acad Sci. 90:5873-5877. Such an algorithm is incorporated into the NBLAST and XBLAST programs of Altschul et al. (1990) J. Mol. Biol. 215:403-410. BLAST nucleotide searches can be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to a protein molecules of the invention. To obtain gapped alignments for comparison purposes, Gapped BLAST can be utilized as described in Altschul et al. (1997) Nucleic Acids Res. 25:3389-3402. Alternatively, PSI-Blast can be used to perform an iterated search which detects distant relationships between molecules (Id.). When utilizing BLAST, Gapped BLAST, and PSI-Blast programs, the default parameters of the respective programs (e.g., XBLAST and NBLAST) can be used. See http://www.ncbi.nlm.nih.gov.

Another preferred, non-limiting example of a mathematical algorithm utilized for the comparison of sequences is the algorithm of Myers and Miller, (1988) CABIOS 4:11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Additional algorithms for sequence analysis are known in the art and include ADVANCE and ADAM as described in Torellis and Robotti (1994) Comput. Appl. Biosci., 10:3-5; and FASTA described in Pearson and Lipman (1988) 85:2444-8. Within FASTA, ktup is a control option that sets the sensitivity and speed of the search. If ktup=2, similar regions in the two sequences being compared are found by looking at pairs of aligned residues; if ktup=1, single aligned amino acids are examined. ktup can be set to 2 or 1 for protein sequences, or from 1 to 6 for DNA sequences. The default if ktup is not specified is 2 for proteins and 6 for DNA. For a further description of FASTA parameters, see http://bioweb.pasteur.fr/docs/man/man/fasta.1.html#sect2.

The percent identity between two sequences can be determined using techniques similar to those described above, with or without allowing gaps. In calculating percent identity, only exact matches are counted. However, conservative substitutions should be considered in evaluating sequences that have a low percent identity with the SGA-1M sequences disclosed herein.

In a specific embodiment, molecules or protein comprising at least 10, 20, 30, 40 or 50 amino acids of SEQ ID NO:4, or at least 10, 20, 30, 40, 50, 75, 100, or 200 amino acids of SEQ ID NO:2 are used in the present invention.

### 5.2.1 Fusion Proteins

SGA-1M gene products can also include fusion proteins comprising an SGA-1M gene product sequence as described above operatively associated to a heterologous, component, e.g., peptide for use in the methods of the invention. Heterologous components can include, but are not limited to sequences which facilitate isolation and purification of fusion protein, or label components. Heterologous components can also include sequences which confer stability to the SGA-1M gene product. Such isolation and label components are well known to those of skill in the art.

The present invention encompasses the use of fusion proteins comprising the protein or fragment thereof encoded for by the SGA-1M gene open reading frames (SEQ ID NO:2 and SEQ ID NO:4) and a heterologous polypeptide (i.e., an unrelated polypeptide or fragment thereof, preferably at least 10, at least 20, at least 30, at least 40, at least 50, at least 60, at least 70, at least 80, at least 90 or at least 100 amino acids of the polypeptide). The fusion can be direct, but may occur through linker sequences. The heterologous polypeptide may be fused to the N-terminus or C-terminus of an SGA-1M gene product.

A fusion protein can comprise an SGA-1M gene product fused to a heterologous signal sequence at its N-terminus. Various signal sequences are commercially available. Eukaryotic heterologous signal sequences include, but art not limited to, the secretory sequences of melittin and human placental alkaline phosphatase (Stratagene; La Jolla, Calif.). Prokaryotic heterologous signal sequences useful in the methods of the invention include, but are not limited to, the phoA secretory signal (Sambrook et al., eds., Molecular Cloning: A Laboratory Manual, 2nd ed, Cold Spring Harbor Laboratory, Cold Spring Harbor Laboratory Press, Cold Spring Harbor, N.Y., 1989) and the protein A secretory signal (Pharmacia Biotech; Piscataway, N.J.).

The SGA-1M protein or fragment thereof encoded for by the SGA-1M open reading frames (SEQ ID NO:2 and SEQ ID NO:4) can be fused to tag sequences, e.g., a hexa-histidine peptide, such as the tag provided in a pQE vector (QIAGEN, Inc., Chatsworth, Calif., 91311), among others, many of which are commercially available for use in the methods of the invention. As described in Gentz et al., 1989, Proc. Natl. Acad Sci. USA, 86:821-824, for instance, hexa-histidine provides for convenient purification of the fusion protein. Other examples of peptide tags are the hemagglutinin “HA” tag, which corresponds to an epitope derived from the influenza hemagglutinin protein (Wilson et al., 1984, Cell, 37:767) and the “flag” tag (Knappik et al., 1994, Biotechniques, 17(4):754-761). These tags are especially useful for purification of recombinantly produced polypeptides of the invention.

Any fusion protein may be readily purified by utilizing an antibody specific or selective for the fusion protein being expressed. For example, a system described by Janknecht et al. allows for the ready purification of non-denatured fusion proteins expressed in human cell lines (Janknecht et al., 1991, Proc. Natl. Acad Sci. USA 88:8972). In this system, the gene of interest is subcloned into a vaccinia recombination plasmid such that the open reading frame of the gene is translationally fused to an amino-terminal tag consisting of six histidine residues. Extracts from cells infected with recombinant vaccinia virus are loaded onto Ni²⁺.nitriloacetic acid-agarose columns and histidine-tagged proteins are selectively eluted with imidazole-containing buffers.

An affinity label can also be fused at its amino terminal to the carboxyl terminal of the protein or fragment thereof encoded for by an SGA-1M open reading frame (SEQ ID NO:2 or SEQ ID NO:4) for use in the methods of the invention. The precise site at which the fusion is made in the carboxyl terminal is not critical. The optimal site can be determined by routine experimentation. An affinity label can also be fused at its carboxyl terminal to the amino terminal of the SGA-1M gene product for use in the methods of the invention.

A variety of affinity labels known in the art may be used, such as, but not limited to, the immunoglobulin constant regions, (Petty, 1996, Metal-chelate affinity chromatography, in Current Protocols in Molecular Biology, Vol. 2, Ed. Ausubel et al., Greene Publish. Assoc. & Wiley Interscience), glutathione S-transferase (GST; Smith, 1993, Methods Mol. Cell Bio. 4:220-229), the E. coli maltose binding protein (Guan et al., 1987, Gene 67:21-30), and various cellulose binding domains (U.S. Pat. Nos. 5,496,934; 5,202,247; 5,137,819; Tomme et al., 1994, Protein Eng. 7:117-123), etc. Other affinity labels may impart fluorescent properties to an SGA-1M gene product, e.g., green fluorescent protein and the like. Other affinity labels are recognized by specific binding partners and thus facilitate isolation by affinity binding to the binding partner which can be immobilized onto a solid support. Some affinity labels may afford the SGA-1M gene product novel structural properties, such as the ability to form multimers. These affinity labels are usually derived from proteins that normally exist as homopolymers. Affinity labels such as the extracellular domains of CD8 (Shiue et al., 1988, J. Exp. Med. 168:1993-2005), or CD28 (Lee et al., 1990, J. Immunol. 145:344-352), or fragments of the immunoglobulin molecule containing sites for interchain disulfide bonds, could lead to the formation of multimers.

As will be appreciated by those skilled in the art, many methods can be used to obtain the coding region of the above-mentioned affinity labels, including but not limited to, DNA cloning, DNA amplification, and synthetic methods. Some of the affinity labels and reagents for their detection and isolation are available commercially.

A preferred affinity label is a non-variable portion of the immunoglobulin molecule. Typically, such portions comprise at least a functionally operative CH2 and CH3 domain of the constant region of an immunoglobulin heavy chain. Fusions are also made using the carboxyl terminus of the Fc portion of a constant domain, or a region immediately amino-terminal to the CH1 of the heavy or light chain. Suitable immunoglobulin-based affinity label may be obtained from IgG-1, -2, -3, or -4 subtypes, IgA, IgE, IgD, or IgM, but preferably IgG1. Preferably, a human immunoglobulin is used when the SGA-1M gene product is intended for in vivo use for humans. Many DNA encoding immunoglobulin light or heavy chain constant regions are known or readily available from cDNA libraries. See, for example, Adams et al., Biochemistry, 1980, 19:2711-2719; Gough et al., 1980, Biochemistry, 19:2702-2710; Dolby et al., 1980, Proc. Natl. Acad Sci. U.S.A., 77:6027-6031; Rice et al., 1982, Proc. Nati. Acad. Sci. U.S.A., 79:7862-7865; Falkner et al., 1982, Nature, 298:286-288; and Morrison et al., 1984, Ann. Rev. Immunol, 2:239-256. Because many immunological reagents and labeling systems are available for the detection of immunoglobulins, the SGA-1M gene product-Ig fusion protein can readily be detected and quantified by a variety of immunological techniques known in the art, such as the use of enzyme-linked immunosorbent assay (ELISA), immunoprecipitation, fluorescence activated cell sorting (FACS), etc. Similarly, if the affinity label is an epitope with readily available antibodies, such reagents can be used with the techniques mentioned above to detect, quantitate, and isolate the SGA-1M gene product containing the affinity label. In many instances, there is no need to develop specific or selective antibodies to the SGA-1M gene product.

A fusion protein can comprise an SGA-1M gene product fused to the Fc domain of an immunoglobulin molecule or a fragment thereof for use in the methods of the invention. A fusion protein can also comprise an SGA-1M gene product fused to the CH2 and/or CH3 region of the Fc domain of an immunoglobulin molecule. Furthermore, a fusion protein can comprise an SGA-1M gene product fused to the CH2, CH3, and hinge regions of the Fc domain of an immunoglobulin molecule (see Bowen et al., 1996, J. Immunol. 156:442-49). This hinge region contains three cysteine residues which are normally involved in disulfide bonding with other cysteines in the Ig molecule. Since none of the cysteines are required for the peptide to function as a tag, one or more of these cysteine residues may optionally be substituted by another amino acid residue, such as for example, serine.

Various leader sequences known in the art can be used for the efficient secretion of the SGA-1M gene product from bacterial and mammalian cells (von Heijne, 1985, J. Mol. Biol. 184:99-105). Leader peptides are selected based on the intended host cell, and may include bacterial, yeast, viral, animal, and mammalian sequences. For example, the herpes virus glycoprotein D leader peptide is suitable for use in a variety of mammalian cells. A preferred leader peptide for use in mammalian cells can be obtained from the V-J2-C region of the mouse immunoglobulin kappa chain (Bernard et al., 1981, Proc. Natl. Acad Sci. 78:5812-5816). Preferred leader sequences for targeting SGA-1M gene product expression in bacterial cells include, but are not limited to, the leader sequences of the E. coli proteins OmpA (Hobom et al., 1995, Dev. Biol. Stand. 84:255-262), Pho A (Oka et al., 1985, Proc. Natl. Acad. Sci 82:7212-16), OmpT (Johnson et al., 1996, Protein Expression 7:104-113), LamB and OmpF (Hoffman & Wright, 1985, Proc. Natl. Acad Sci. USA 82:5107-51 11), β-lactamase (Kadonaga et al., 1984, J. Biol. Chem. 259:2149-54), enterotoxins (Morioka-Fujimoto et al., 1991, J. Biol. Chem. 266:1728-32), and the Staphylococcus aureus protein A (Abrahmsen et al., 1986, Nucleic Acids Res. 14:7487-7500), and the B. subtilis endoglucanase (Lo et al., Appl. Environ. Microbiol. 54:2287-2292), as well as artificial and synthetic signal sequences (MacIntyre et al., 1990, Mol. Gen. Genet. 221:466-74; Kaiser et al, 1987, Science, 235:312-317).

A fusion protein can comprise an SGA-1M gene product and a cell permeable peptide, which facilitates the transport of a protein or polypeptide across the plasma membrane for use in the methods of the invention. Examples of cell permeable peptides include, but are not limited to, peptides derived from hepatitis B virus surface antigens (e.g., the PreS2- domain of hepatitis B virus surface antigens), herpes simplex virus VP22, antennapaedia, 6H, 6K, and 6R. See, e.g., Oess et al., 2000, Gene Ther. 7:750-758, DeRossi et al., 1998, Trends Cell Biol 8(2):84-7, and Hawiger, 1997, J. Curr Opin Immunol 9(2):189-94.

Fusion proteins can be produced by standard recombinant DNA techniques or by protein synthetic techniques, e.g., by use of a peptide synthesizer. For example, a nucleic acid molecule encoding a fusion protein can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, e.g., Current Protocols in Molecular Biology, Ausubel et al., eds., John Wiley & Sons, 1992).

The nucleotide sequence coding for a fusion protein can be inserted into an appropriate expression vector, i.e., a vector which contains the necessary elements for the transcription and translation of the inserted protein-coding sequence. The expression of a fusion protein may be regulated by a constitutive, inducible or tissue-specific or -selective promoter. It will be understood by the skilled artisan that fusion proteins, which can facilitate solubility and/or expression, and can increase the in vivo half-life of the protein or fragment thereof encoded for by an SGA-1M ORF (SEQ ID NO:2 or SEQ ID NO:4) and thus are useful in the methods of the invention. The SGA-1M gene products or peptide fragments thereof, or fusion proteins can be used in any assay that detects or measures SGA-1M gene products or in the calibration and standardization of such assay.

The methods of invention encompass the use of SGA-1M gene products or peptide fragments thereof, which may be produced by recombinant DNA technology using techniques well known in the art. Thus, methods for preparing the SGA-1M gene polypeptides and peptides of the invention by expressing nucleic acid containing SGA-1M gene sequences are described herein. Methods which are well known to those skilled in the art can be used to construct expression vectors containing SGA-1M gene product coding sequences (including but not limited to SEQ ID NO:2 and SEQ ID NO:4) and appropriate transcriptional and translational control signals. These methods include, for example, in vitro recombinant DNA techniques, synthetic techniques, and in vivo genetic recombination. See, for example, the techniques described in Sambrook et al., 1989, supra, and Ausubel et al., 1989, supra. Alternatively, RNA capable of encoding SGA-1M gene product sequences may be chemically synthesized using, for example, synthesizers (see e.g., the techniques described in Oligonucleotide Synthesis, 1984, Gait, M. J. ed., IRL Press, Oxford).

### 5.2.2 Expression Systems

A variety of host-expression vector systems may be utilized to express the SGA-1M gene coding sequences for use in the methods of the invention. Such host-expression systems represent vehicles by which the coding sequences of interest may be produced and subsequently purified, but also represent cells which may, when transformed or transfected with the appropriate nucleotide coding sequences, exhibit the SGA-1M gene product of the invention in situ. These include but are not limited to microorganisms such as bacteria (e.g., E. coli, B. subtilis) transformed with recombinant bacteriophage DNA, plasmid DNA or cosmid DNA expression vectors containing SGA-1M gene product coding sequences; yeast (e.g., Saccharomyces, Pichia) transformed with recombinant yeast expression vectors containing the SGA-1M gene product coding sequences; insect cell systems infected with recombinant virus expression vectors (e.g., baculovirus) containing the SGA-1M gene product coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing SGA-1M gene product coding sequences; or mammalian cell systems (e.g., COS, CHO, BHK, 293, 3T3) harboring recombinant expression constructs containing promoters derived from the genome of mammalian cells (e.g., metallothionein promoter) or from mammalian viruses (e.g., the adenovirus late promoter; the vaccinia virus 7.5K promoter).

In bacterial systems, a number of expression vectors may be advantageously selected depending upon the use intended for the SGA-1M gene product being expressed. For example, when a large quantity of such a protein is to be produced, for the generation of pharmaceutical compositions of SGA-1M protein or for raising antibodies to SGA-1M protein, vectors which direct the expression of high levels of fusion protein products that are readily purified may be desirable. Such vectors include, but are not limited, to the E. coli expression vector pUR278 (Ruther et al., 1983, EMBO J. 2:1791), in which the SGA-1M gene product coding sequence may be ligated individually into the vector in frame with the lac Z coding region so that a fusion protein is produced; pIN vectors (Inouye & Inouye, 1985, Nucleic Acids Res. 13:3101; Van Heeke & Schuster, 1989, J. Biol. Chem. 264:5503); and the like. pGEX vectors may also be used to express foreign polypeptides as fusion proteins with glutathione S-transferase (GST). In general, such fusion proteins are soluble and can easily be purified from lysed cells by adsorption and binding to a matrix glutathione-agarose beads followed by elution in the presence of free glutathione. The pGEX vectors ate designed to include, e.g.,thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The SGA-1M gene coding sequence may be cloned individually into non-essential regions (for example the polyhedrin gene) of the virus and placed under control of an AcNPV promoter (for example the polyhedrin promoter). Successful insertion of SGA-1M gene coding sequence will result in inactivation of the polyhedrin gene and production of non-occluded recombinant virus (i.e., virus lacking the proteinaceous coat coded for by the polyhedrin gene). These recombinant viruses are then used to infect Spodoptera frugiperda cells in which the inserted gene is expressed (e.g., see Smith et al., 1983, J. Virol. 46:584; Smith, U.S. Pat. No. 4,215,051).

In mammalian host cells, a number of viral-based expression systems may be utilized. In cases where an adenovirus is used as an expression vector, the SGA-1M gene coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing SGA-1M gene product in infected hosts. (See, e.g., Logan & Shenk, 1984, Proc. Natl. Acad Sci. USA 81:3655). Specific initiation signals may also be required for efficient translation of inserted SGA-1M gene product coding sequences. These signals include the ATG initiation codon and adjacent sequences. In cases where an entire SGA-1M gene, including its own initiation codon and adjacent sequences, is inserted into the appropriate expression vector, no additional translational control signals may be needed. However, in cases where only a portion of the SGA-1M gene coding sequence is inserted, exogenous translational control signals, including, perhaps, the ATG initiation codon, must be provided. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (See Bittner et al., 1987, Methods in Enzymol. 153:516).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERO, BHK, HeLa, COS, MDCK, 293, 3T3, WI38, and in particular, breast cancer cell lines such as, for example, BT483, Hs578T, HTB26, BT20 and T47D, and normal mammary gland cell line such as, for example, CRL7030 and Hs578Bst.

For long-term, high-yield production of recombinant proteins, stable expression is preferred. For example, cell lines which stably express the SGA-1M gene product may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the SGA-1M gene product. Such engineered cell lines may be particularly useful in screening and evaluation of compounds that affect the endogenous activity of the SGA-1M gene product.

A number of selection systems may be used, including but not limited to the herpes simplex virus thymidine kinase (Wigler et al., 1977, Cell 11:223), hypoxanthine-guanine phosphoribosyltransferase (Szybalska & Szybalski, 1962, Proc. Natl. Acad Sci. USA 48:2026), and adenine phosphoribosyltransferase (Lowy et al., 1980, Cell 22:817) genes can be employed in tk⁻, hgprt⁻ or aprt⁻ cells, respectively. Also, antimetabolite resistance can be used as the basis of selection for the following genes: dhfr, which confers resistance to methotrexate (Wigler et al., 1980, Proc Natl. Acad Sci. USA 77:3567; O'Hare et al., 1981, Proc. Natl. Acad Sci. USA 78:1527); gpt, which confers resistance to mycophenolic acid (Mulligan & Berg, 1981, Proc. Natl. Acad Sci. USA 78:2072); neo, which confers resistance to the aminoglycoside G-418 (Colberre-Garapin et al., 1981, J. Mol. Biol. 150: 1); and hygro, which confers resistance to hygromycin (Santerre et al., 1984, Gene 30:147).

In another embodiment, the expression characteristics of SGA-1M within a cell, cell line or microorganism may be modified by inserting a DNA regulatory element heterologous to the SGA-1M gene into the genome of a cell, stable cell line or cloned microorganism such that the inserted regulatory element is operatively linked with the SGA-1M gene and controls, modulates or activates transcription of the SGA-1M gene. For example, an endogenous SGA-1M gene which is normally “transcriptionally silent”, i.e., an endogenous SGA-1M gene which is normally not expressed, or is expressed only at very low levels in a cell line or microorganism, may be activated by inserting a regulatory element which is capable of promoting the expression of a normally expressed gene product in that cell line or microorganism. Alternatively, a transcriptionally silent, endogenous SGA-1M gene may be activated by insertion of a promiscuous regulatory element that works across cell types.

A heterologous regulatory element may be inserted into a stable cell line or cloned microorganism, such that it is operatively linked with and activates expression of an endogenous SGA-1M gene, using techniques, such as targeted homologous recombination, which are well known to those of skill in the art, and described e.g., in Chappel, U.S. Pat. No. 5,272,071; PCT publication No. WO 91/06667, published May 16, 1991; Skoultchi, U.S. Pat. No. 5,981,214; Treco et al., U.S. Pat. No. 5,968,502 and PCT publication No. WO 94/12650, published Jun. 9, 1994. Alternatively, non-targeted, e.g., non-homologous recombination techniques which are well-known to those of skill in the art and described, e.g., in PCT publication No. WO 99/15650, published Apr. 1, 1999, may be used.

### 5.2.3 SGA-1M Transgenic Animals

The SGA-1M gene products can also be expressed in transgenic animals. Animals of any species, including, but not limited to, mice, rats, rabbits, guinea pigs, sheep, pigs, micro-pigs, goats, and non-human primates, e.g., baboons, monkeys, and chimpanzees may be used to generate SGA-1M transgenic animals.

Transgenic animals that over- or mis-express an SGA-1M gene product may be used in any of the methods of the invention. For example transgenic animals may be used to study the in vivo effects of enhanced expression levels of SGA-1M and the onset, diagnosis or prognosis of cancer. Transgenic animals would be useful to screen antagonists or agonists of SGA-1M. Transgenic animals could be used to screen the in vivo effects of anti-sense or ribozyme therapeutic molecules in the treatment of cancer. Transgenic animals could be used to screen for methods of vaccinating against cancer using an SGA-1M gene product or a portion thereof.

Further, SGA-1M knock out animals are also useful in the methods of the invention. For example, animals with disruptions in only SGA-1M(A) or SGA-1M(B) can be useful in assessing the relative contribution of each of these gene products to the cancer state, as well as assessing the positive effect of a cancer therapeutic candidate.

For over- or mis-expression of an SGA-1M gene product, any technique known in the art may be used to introduce the SGA-1M gene product into animals to produce the founder lines of transgenic animals. Such techniques include, but are not limited to pronuclear microinjection (Hoppe and Wagner, 1989, U.S. Pat. No. 4,873,191); retrovirus mediated gene transfer into germ lines (Van der Putten et al., 1985, Proc. Natl. Acad Sci. USA 82:6148); gene targeting in embryonic stem cells (Thompson et al., 1989, Cell 56:313); electroporation of embryos (Lo, 1983, Mol Cell. Biol. 3:1803); and sperm-mediated gene transfer (Lavitrano et al., 1989, Cell 57:717); etc. For a review of such techniques, see Gordon, 1989, Transgenic Animals, Intl. Rev. Cytol. 115:171.

The methods of the invention provide for the use of transgenic animals that carry the SGA-1M transgene in all their cells, as well as animals which carry the transgene in some, but not all their cells, i.e., mosaic animals.

The transgene may be integrated as a single transgene or in concatamers, e.g., head-to-head tandems or head-to-tail tandems. The transgene may also be selectively introduced into and activated in a particular cell type by following, for example, the teaching of Lasko et al. (Lasko et al., 1992, Proc. Natl. Acad Sci. USA 89:6232). The regulatory sequences required for such a cell-type specific activation will depend upon the particular cell type of interest, and will be apparent to those of skill in the art.

When it is desired that the SGA-1M transgene be integrated into the chromosomal site of the endogenous SGA-1M gene, for example to disrupt the expression of SGA-1M(A) or SGA-1M(B), gene targeting is preferred. Briefly, when such a technique is to be utilized, vectors containing some nucleotide sequences homologous to the endogenous SGA-1M gene are designed for the purpose of integrating, via homologous recombination with chromosomal sequences, into and partially or wholly disrupting the function of the nucleotide sequence of the endogenous SGA-1M gene. The transgene may also be selectively introduced into a particular cell type, thus inactivating the endogenous SGA-1M gene in only that cell type, by following, for example, the teaching of Gu et al. (Gu et al., 1994, Science 265:103). The regulatory sequences required for such a cell-type specific inactivation will depend upon the particular cell type of interest, and will be apparent to those of skill in the art.

Methods for the production of single-copy transgenic animals with chosen sites of integration are also well known to those of skill in the art. See, for example, Bronson et al. (Bronson, S. K. et al., 1996, Proc. Natl. Acad. Sci. USA 93:9067).

Once transgenic animals have been generated, the expression of the recombinant SGA-1M gene may be assayed utilizing standard techniques. Initial screening may be accomplished by Southern blot analysis or PCR techniques to analyze animal tissues to assay whether integration of the transgene has taken place. The level of mRNA expression of the transgene in the tissues of the transgenic animals may also be assessed using techniques which include but are not limited to Northern blot analysis of tissue samples obtained from the animal, in situ hybridization analysis, and RT-PCR. Samples of SGA-1M gene-expressing tissue, may also be evaluated immunocytochemically using antibodies specific or selective for the SGA-1M(A) and/or SGA-1M(B) gene product.

### 5.3. Antibodies to SGA-1M Gene Products

The methods of the present invention encompass the use of antibodies or fragments thereof capable of specifically or selectively recognizing one or more SGA-1M gene product epitopes or epitopes of conserved variants or peptide fragments of the SGA-1M gene products. Such antibodies may include, but are not limited to, polyclonal antibodies, monoclonal antibodies (mAbs), humanized or chimeric antibodies, single chain antibodies, Fab fragments, F(ab′)₂ fragments, Fv fragments, fragments produced by a Fab expression library, anti-idiotypic (anti-Id) antibodies, and epitope-binding fragments of any of the above.

Such antibodies may be used, for example, in the detection of an SGA-1M gene product in an biological sample and may, therefore, be utilized as part of a diagnostic or prognostic technique whereby patients may be tested for abnormal levels of SGA-1M gene products, and/or for the presence of abnormal forms of the such gene products. Such antibodies may also be included as a reagent in a kit for use in a diagnostic or prognostic technique. Such antibodies may also be utilized in conjunction with, for example, compound screening methods, as described, below, in Section 5.5, for the evaluation of the effect of test compounds on SGA-1 M gene product levels and/or activity. Additionally, such antibodies can be used in conjunction with the gene therapy techniques described, below, in Section 5.6.4, to, for example, evaluate the normal and/or engineered SGA-1M-expressing cells prior to their introduction into the patient.

Antibodies to the SGA-1M gene product may additionally be used in a method for the inhibition of SGA-1M gene product activity. Thus, such antibodies may, therefore, be utilized as part of cancer treatment methods.

Described herein are methods for the production of antibodies or fragments thereof. Any of such antibodies or fragments thereof may be produced by standard immunological methods or by recombinant expression of nucleic acid molecules encoding the antibody or fragments thereof in an appropriate host organism.

For the production of antibodies against an SGA-1M gene product, various host animals may be immunized by injection with an SGA-1M gene product, or a portion thereof. Such host animals may include but are not limited to rabbits, mice, and rats, to name but a few. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum.

Polyclonal antibodies are heterogeneous populations of antibody molecules derived from the sera of animals immunized with an antigen, such as an SGA-1M gene product, or an antigenic functional derivative thereof. For the production of polyclonal antibodies, host animals such as those described above, may be immunized by injection with SGA-1M gene product supplemented with adjuvants as also described above.

Monoclonal antibodies, which are homogeneous populations of antibodies to a particular antigen, may be obtained by any technique which provides for the production of antibody molecules by continuous cell lines in culture. These include, but are not limited to, the hybridoma technique of Kohler and Milstein, (1975, Nature 256:495; and U.S. Pat. No. 4,376,110), the human B-cell hybridoma technique (Kosbor et al., 1983, Immunology Today 4:72; Cole et al., 1983, Proc. Natl. Acad Sci. USA 80:2026), and the EBV-hybridoma technique (Cole et al., 1985, Monoclonal Antibodies And Cancer Therapy, Alan R. Liss, Inc., pp. 77). Such antibodies may be of any immunoglobulin class including IgG, IgM, IgE, IgA, IgD and any subclass thereof. The hybridoma producing the mAb of this invention may be cultivated in vitro or in vivo. Production of high titers of mAbs in vivo makes this the presently preferred method of production.

Techniques developed for the production of“chimeric antibodies” (Morrison et al., 1984, Proc. Natl. Acad. Sci., 81, 6851-6855; Neuberger et al., 1984, Nature 312, 604-608; Takeda et al., 1985, Nature 314, 452-454) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable region derived from a murine mAb and a human immunoglobulin constant region. (See, e.g., Cabilly et al., U.S. Pat. No. 4,816,567; and Boss et al., U.S. Pat. No. 5,816,397). The invention thus contemplates chimeric antibodies that are specific or selective for an SGA-1M gene product.

Examples of techniques that have been developed for the production of humanized antibodies are known in the art. (See, e.g., Queen, U.S. Pat. No. 5,585,089 and Winter, U.S. Pat. No. 5,225,539) An immunoglobulin light or heavy chain variable region consists of a “framework” region interrupted by three hypervariable regions, referred to as complementarity-determining regions (CDRs). The extent of the framework region and CDRs have been precisely defined (see, “Sequences of Proteins of Immunological Interest”, Kabat, E. et al., U.S. Department of Health and Human Services (1983). Briefly, humanized antibodies are antibody molecules from non-human species having one or more CDRs from the non-human species and framework regions from a human immunoglobulin molecule. The invention includes the use of humanized antibodies that are specific or selective for an SGA-1M gene product in the methods of the invention.

The methods of the invention encompasses the use of an antibody or derivative thereof comprising a heavy or light chain variable domain, said variable domain comprising (a) a set of three complementarity-determining regions (CDRs), in which said set of CDRs are from a monoclonal antibody to a gene product encoded for by an SGA-1M open reading frame (SEQ ID NO:2 or SEQ ID NO:4), and (b) a set of four framework regions, in which said set of framework regions differs from the set of framework regions in the monoclonal antibody to a gene product encoded by an SGA-1M open reading frame (SEQ ID NO:2 or SEQ ID NO:4), and in which said antibody or derivative thereof immunospecifically binds to the gene product encoded for by the SGA-1M gene sequence. Preferably, the set of framework regions is from a human monoclonal antibody, e.g., a human monoclonal antibody that does not bind the gene product encoded for by the SGA-1M gene sequence.

Phage display technology can be used to increase the affinity of an antibody to an SGA-1M gene product. This technique would be useful in obtaining high affinity antibodies to an SGA-1M gene product that could be used for the diagnosis and prognosis of a subject with cancer. The technology, referred to as affinity maturation, employs mutagenesis or CDR walking and re-selection using the SGA-1M gene product antigen to identify antibodies that bind with higher affinity to the antigen when compared with the initial or parental antibody (see, e.g., Glaser et al., 1992, J. Immunology 149:3903). Mutagenizing entire codons rather than single nucleotides results in a semi-randomized repertoire of amino acid mutations. Libraries can be constructed consisting of a pool of variant clones each of which differs by a single amino acid alteration in a single CDR and which contain variants representing each possible amino acid substitution for each CDR residue. Mutants with increased binding affinity for the antigen can be screened by contacting the immobilized mutants with labeled antigen. Any screening method known in the art can be used to identify mutant antibodies with increased avidity to the antigen (e.g., ELISA) (See Wu et al., 1998, Proc Natl. Acad Sci. USA 95:6037; Yelton et al., 1995, J. Immunology 155:1994). CDR walking which randomizes the light chain is also possible (See Schier et al, 1996, J. Mol. Bio. 263:551).

Alternatively, techniques described for the production of single chain antibodies (U.S. Pat. No. 4,946,778; Bird, 1988, Science 242:423; Huston et al., 1988, Proc. Natl. Acad Sci. USA 85:5879; and Ward et al., 1989, Nature 334:544) can be adapted to produce single chain antibodies against SGA-1M gene products. Single chain antibodies are formed by linking the heavy and light chain fragments of the Fv region via an amino acid bridge, resulting in a single chain polypeptide. Techniques for the assembly of functional Fv fragments in E. coli may also be used (Skerra et al, 1988, Science 242:1038).

The methods of the invention include using an antibody to an SGA-1M polypeptide, peptide or other derivative, or analog thereof that is a bispecific antibody (see generally, e.g., Fanger and Drakeman, 1995, Drug News and Perspectives 8:133-137). Bispecific antibodies can be used for example to treat or prevent cancer in a subject that expresses elevated levels of an SGA-1M gene product. Such a bispecific antibody is genetically engineered to recognize both (1) an epitope and (2) one of a variety of “trigger” molecules, e.g., Fc receptors on myeloid cells, and CD3 and CD2 on T cells, that have been identified as being able to cause a cytotoxic T-cell to destroy a particular target. Such bispecific antibodies can be prepared either by chemical conjugation, hybridoma, or recombinant molecular biology techniques known to the skilled artisan.

Antibody fragments which recognize specific epitopes may be generated by known techniques. For example, such fragments include but are not limited to: the F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed (Huse et al., 1989, Science 246:1275-1281) to allow rapid and easy identification of monoclonal Fab fragments with the desired specificity.

### 5.3.1 SGA-1M Antibody Conjugates

The SGA-1M antibodies of the present invention can be conjugated to therapeutic agents for use in the methods of the present invention. Particular suitable moieties for conjugation to anti-CD70 antibodies of the invention are chemotherapeutic or cytotoxic agents, pro-drug converting enzymes, radioactive isotopes or compounds, or toxins.

In certain embodiments, the SGA-1M antibodies of the invention are conjugated to a radionuclide (e.g., alpha-emitters such as, for example, ²¹²Bi, ²¹¹At, or beta-emitters such as, for example, ¹³¹I, ⁹⁰Y, or ⁶⁷Cu).

In other embodiments, the SGA-1M antibodies of the invention are conjugated to non-classical therapeutic agents such as toxins. Such toxins include, but are not limited to, abrin, ricin A, pseudomonas exotoxin, or diphtheria toxin.

In yet other embodiments, the SGA-1M antibodies of the invention are fused or conjugated to a pro-drug converting enzyme. Exemplary pro-drug converting enzymes are carboxypeptidase G2, beta-glucuronidase, penicillin-V-amidase, penicillin-G-arnidase, β-lactamase, β-glucosidase, nitroreductase and carboxypeptidase A. Such fusion proteins and conjugated are useful therapeutic agents when co-administered with a pro-drug.

In other embodiments, the SGA-1M antibodies of the invention are conjugated to cytotoxic agents. Examples of cytotoxic agents that can be conjugated to an anti-SGA-1M antibody include alkylating agents, anthracyclines, antibiotics, antifolates, antimetabolites, antitubulin agents, auristatins, chemotherapy sensitizers, DNA minor groove binders, DNA replication inhibitors, duocarmycins, etoposides, fluorinated pyrimidines, lexitropsins, nitrosoureas, platinols, purine antimetabolites, puromycins, radiation sensitizers, steroids, taxanes, topoisomerase inhibitors, vinca alkaloids, purine antagonists, and dihydrofolate reductase inhibitors. In more specific embodiments, the cytotoxic agent can be an androgen, anthramycin (AMC), asparaginase, 5-azacytidine, azathioprine, bleomycin, busulfan, buthionine sulfoximine, camptothecin, carboplatin, carmustine (BSNU), CC-1065, chlorambucil, cisplatin, colchicine, cyclophosphamide, cytarabine, cytidine arabinoside, cytochalasin B, dacarbazine, dactinomycin (formerly actinomycin), daunorubicin, decarbazine, docetaxel, doxorubicin, an estrogen, 5-fluordeoxyuridine, 5-fluorouracil, gramicidin D, hydroxyurea, idarubicin, ifosfamide, irinotecan, lomustine (CCNU), mechlorethamine, melphalan, 6-mercaptopurine, methotrexate, mithramycin, mitomycin C, mitoxantrone, nitroimidazole, paclitaxel, plicamycin, procarbizine, streptozotocin, tenoposide, 6-thioguanine, thioTEPA, topotecan, vinblastine, vincristine, vinorelbine, VP-16, VM-26, azothioprine, mycophenolate mofetil, methotrexate, acyclovir, gangcyclovir, zidovudine, vidarabine, ribavarin, azidothymidine, cytidine arabinoside, amantadine, dideoxyuridine, iododeoxyuridine, poscamet, and trifluridine.

In certain preferred embodiments, the cytotoxic agent conjugated to an anti-SGA-1M antibody is selected from the group consisting of an enediyne, a lexitropsin, a duocarmycin, a taxane, a puromycin, a dolastatin, an auristatin, a maytansinoid, and a vinca alkaloid. In certain, more specific embodiments, the cytotoxic agent is paclitaxel, docetaxel, CC-1065, SN-38, topotecan, morpholino-doxorubicin, rhizoxin, cyanomorpholino-doxorubicin, dolastatin-1 0, echinomycin, combretastatin, calicheamicin, maytansine, DM-1, auristatin E, AEB, AEVB, AEFP, MMAE, or netropsin. The structures of AEB, AEVB, AEFP and MMAE and methods of making conjugating these cytotoxic agents to an antibody are described in U.S. provisional application No. 60/400,403, filed Jul. 31, 2002, and 60/427,897, filed Nov. 20, 2002, each of which is incorporated herein in its entirety.

In other preferred embodiments, the cytotoxic agent of an anti-SGA-1M antibody-cytotoxic agent conjugate is an anti-tubulin agent. In more specific embodiments, the cytotoxic agent is selected from the group consisting of a vinca alkaloid, a podophyllotoxin, a taxane, a baccatin derivative, a cryptophysin, a maytansinoid, a combretastatin, a dolastatin and an auristatin. In more specific embodiments, the cytotoxic agent is vincristine, vinblastine, vindesine, vinorelbine, VP-16, camptothecin, paclitaxel, docetaxel, epithilone A, epithilone B, nocodazole, colchicine, colcimid, estramustine, cemadotin, discodermolide, maytansine, DM-1, AEFP, auristatin E, AEB, AEVB, AEFP, MMAE or eleutherobin.

In certain specific embodiments, the anti-SGA-1M antibody of an anti-SGA-1M antibody-cytotoxic agent conjugate of the invention is conjugated to the cytotoxic agent via a linker, wherein the linker is peptide linker. In specific embodiments, the anti-SGA-1M antibody of an anti-SGA-1M antibody-cytotoxic agent conjugate of the invention is conjugated to the cytotoxic agent via a linker, wherein the linker is a valine-citrulline (val-cit) linker, a phenylalanine-lysine (phe-lys) linker, a hydrazone linker, a thioether linker, or a disulfide linker. In certain embodiments, the anti-SGA-1M antibody of an anti-SGA-1M antibody-cytotoxic agent conjugate of the invention is conjugated to the cytotoxic agent via a peptide linker.

In certain embodiments, the anti-SGA-1M antibody of an anti-SGA-1M antibody-cytotoxic agent conjugate of the invention is conjugated to the cytotoxic agent via a linker, wherein the linker is hydrolyzable at a pH of less than 5.5. In a specific embodiment the linker is hydrolyzable at a pH of less than 5.0.

In certain embodiments, the anti-SGA-1M antibody of an anti-SGA-1M antibody-cytotoxic agent conjugate of the invention is conjugated to the cytotoxic agent via a linker, wherein the linker is cleavable by a protease. In a specific embodiment, the protease is a lysosomal protease. In other specific embodiments, the protease is, inter alia, a membrane-associated protease, an intracellular protease, or an endosomal protease.

### 5.4. Uses of the SGA-1M Gene, Gene Products, and Antibodies

In various embodiments, the present invention provides various uses of the SGA-1M gene, the SGA-1M(A) and SGA-1M(B) polypeptides and peptide fragments thereof, and of antibodies directed against the SGA-1M(A) and SGA-1M(B) polypeptides and peptide fragments. Such uses include, for example, prognostic and diagnostic evaluation of cancer, and the identification of subjects with a predisposition to a cancer, as described, below. The invention also includes methods of treating and preventing cancer. The invention includes methods of vaccinating against cancer. The methods of the invention can be used for the treatment, prevention, vaccination, diagnosis, staging and or prognosis of any cancer, or tumor, for example, but not limited to, any of the tumors or cancers listed below in Table 1.

Malignancies and related disorders, cells of which type can be tested in vitro (and/or in vivo), and upon observing the appropriate assay result, treated according to the methods of the present invention, include but are not limited to those listed in Table 1 (for a review of such disorders, see Fishman et al., 1985, Medicine, 2d Ed., J. B. Lippincott Co., Philadelphia):

TABLE 1
MALIGNANCIES AND RELATED DISORDERS

| Leukemia | | | |
|---|---|---|---|
| | acute leukemia | | |
| | | acute lymphocytic leukemia | |
| | | acute myelocytic leukemia | |
| | | | myeloblastic |
| | | | promyelocytic |
| | | | myelomonocytic |
| | | | monocytic |
| | | | erythroleukemia |

| | chronic leukemia | |
|---|---|---|
| | | chronic myelocytic (granulocytic) leukemia |
| | | chronic lymphocytic leukemia |
| Polycythemia vera | | |
| Lymphoma | | |
| | Hodgkin's disease | |
| | non-Hodgkin's disease | |
| Multiple myeloma | | |
| Waldenström's macroglobulinemia | | |
| Heavy chain disease | | |
| Solid tumors | | |
| | sarcomas and carcinomas | |
| | | fibrosarcoma |
| | | myxosarcoma |
| | | liposarcoma |
| | | chondrosarcoma |
| | | osteogenic sarcoma |
| | | chordoma |
| | | angiosarcoma |
| | | endotheliosarcoma |
| | | lymphangiosarcoma |
| | | lymphangioendotheliosarcoma |
| | | synovioma |
| | | mesothelioma |
| | | Ewing's tumor |
| | | leiomyosarcoma |
| | | rhabdomyosarcoma |
| | | colon carcinoma |
| | | pancreatic cancer |
| | | breast cancer |
| | | ovarian cancer |
| | | prostate cancer |
| | | squamous cell carcinoma |
| | | basal cell carcinoma |
| | | adenocarcinoma |
| | | sweat gland carcinoma |
| | | sebaceous gland carcinoma |
| | | papillary carcinoma |
| | | papillary adenocarcinomas |
| | | cystadenocarcinoma |
| | | medullary carcinoma |
| | | bronchogenic carcinoma |
| | | renal cell carcinoma |
| | | hepatoma |
| | | bile duct carcinoma |
| | | choriocarcinoma |
| | | seminoma |
| | | embryonal carcinoma |
| | | Wilms' tumor |
| | | cervical cancer |
| | | testicular tumor |
| | | lung carcinoma |
| | | small cell lung carcinoma |
| | | bladder carcinoma |
| | | epithelial carcinoma |
| | | glioma |
| | | astrocytoma |
| | | medulloblastoma |
| | | craniopharyngioma |
| | | ependymoma |
| | | pinealoma |
| | | hemangioblastoma |
| | | acoustic neuroma |
| | | oligodendroglioma |
| | | menangioma |
| | | melanoma |
| | | neuroblastoma |
| | | retinoblastoma |

In a preferred embodiment the methods of the invention are directed at diagnosis, prognosis, treatment and prevention of breast cancer (e.g., breast adenocarcinoma). In other embodiments, the cancer is ovarian cancer, skin cancer (e.g., melanoma), a cancer of the lymphoid system (e.g., lymphoma), thyroid cancer (e.g., thyroid carcinoma), pancreatic cancer (e.g., pancreas adenocarcinoma), stomach cancer (e.g., stomach adenocarcinoma), or lung cancer (e.g., lung adenocarcinoma).

In a preferred embodiment the methods of the invention are directed at diagnosis, prognosis, treatment and prevention of a carcinoma (e.g., thyroid carcinoma). In another preferred embodiment the methods of the invention are directed at diagnosis, prognosis, treatment and prevention of an adenocarcinoma (e.g., breast adenocarcinoma, pancreas adenocarcinoma, stomach adenocarcinoma and lung adenocarcinoma).

In certain embodiments, the present invention is not directed at diagnosis, prognosis, treatment and prevention of kidney cancer, rectal cancer, prostate cancer, cancer of the small intestine, liver cancer, or cancer colon cancer.

The invention further provides for screening assays to identify antagonists or agonists of the SGA1-M gene or gene product. Thus, the invention encompasses methods for identify molecules which up regulate or down regulate expression of the SGA-1M gene.

The nucleic acid molecules, proteins, protein homologs, and antibodies described herein can be used in one or more of the following methods: a) screening assays; b) detection assays (e.g., chromosomal mapping, tissue typing); c) predictive medicine (e.g., diagnostic assays, prognostic assays, monitoring clinical trials, and pharnacogenomics); and d) methods of treatment (e.g., therapeutic and prophylactic). For example, an SGA-1M gene product can be used to modulate (i) cellular proliferation; (ii) cellular differentiation; and/or (iii) cellular adhesion. Isolated nucleic acid molecules which encode the SGA-1M gene or a fragment or an open reading frame thereof can be used to express proteins (e.g., via a recombinant expression vector in a host cell in gene therapy applications), to detect MRNA (e.g., in a biological sample) or a genetic lesion, and to modulate activity of an SGA-1M polypeptide. In addition, an SGA-1M gene product can be used to screen drugs or compounds which modulate activity or expression of the SGA-1M gene product as well as to treat disorders characterized by insufficient or excessive production of the SGA-1M gene product or production of a form the SGA-1M gene product which has decreased or aberrant activity compared to the wild type protein. In addition, the antibodies that specifically or selectively bind to an SGA-1M gene product can be used to detect, isolate, and modulate activity of the SGA-1M gene product.

In one embodiment, the present invention provides a variety of methods for the diagnostic and prognostic evaluation of cancer, including breast cancer. Such methods may, for example, utilize reagents such as the SGA-1M gene nucleotide sequences described in Sections 5. 1, and antibodies directed against SGA-1M gene products, including peptide fragments thereof, as described, above, in Section 5.2. Specifically, such reagents may be used, for example, for: (1) the detection of the presence of SGA-1M gene mutations, or the detection of either over- or under-expression of SGA-1M gene mRNA, preneoplastic or neoplastic, relative to normal cells or the qualitative or quantitative detection of other allelic forms of SGA-1M transcripts which may correlate with breast cancer or susceptibility toward neoplastic changes, and (2) the detection of an over-abundance of an SGA-1M gene product relative to the non-disease state or relative to a predetermined non-cancerous standard or the presence of a modified (e.g., less than full length) SGA-1M gene product which correlates with a neoplastic state or a progression toward neoplasia or metastasis.

The methods described herein may be performed, for example, by utilizing pre-packaged diagnostic test kits comprising at least one specific or selective SGA-1M gene nucleic acid or anti-SGA-1M(A) or anti-SGA-1M(B) antibody reagent described herein, which may be conveniently used, e.g., in clinical settings or in home settings, to diagnose patients exhibiting preneoplastic or neoplastic abnormalities, and to screen and identify those individuals exhibiting a predisposition to such neoplastic changes.

Nucleic acid-based detection techniques are described, below, in Section 5.4.1. Peptide detection techniques are described, below, in Section 5.4.2.

### 5.4.1. Detection of SGA-1M Gene Nucleic Acid Molecules

In a preferred embodiment, the invention involves methods to assess quantitative and qualitative aspects of SGA-1M gene expression. In one example the increased expression of an SGA-1M gene or gene product indicates a predisposition for the development of cancer. Alternatively, enhanced expression levels of an SGA-1M gene or gene product can indicate the presence of cancer in a subject or the risk of metastasis of said cancer in said subject. Techniques well known in the art, e.g., quantitative or semi-quantitative RT PCR or Northern blot, can be used to measure expression levels of SGA-1M. Methods which describe both qualitative and quantitative aspects of SGA-1M gene or gene product expression are described in detail in the examples infra. The measurement of SGA-1M gene expression levels can include measuring naturally occurring SGA-LM transcripts and variants thereof as well as non-naturally occurring variants thereof, however for the diagnosis and/or prognosis of cancer in a subject the SGA-1M gene product is preferably a naturally occurring SGA-1M gene product or variant thereof. Thus, the invention relates to methods of diagnosing or predicting cancer in a subject by measuring the expression of the SGA-1M gene in a subject. For example the increased level of mRNA encoded for by the SGA-1M cDNA (SEQ ID NO:1), or other gene product, as compared to a non-cancerous sample or a non-canctrous predetermined standard would indicate the presence of cancer in said subject or the increased risk of developing cancer in said subject.

In another example the increased level of mRNA encoded for by the SGA-1M cDNA (SEQ ID NO:1), or other gene product, as compared to a non-cancerous sample or a non-cancerous predetermined standard would indicate the risk of metastasis of the cancer in said subject or the likelihood of a poor prognosis in said subject.

In another example, RNA from a cell type or tissue known, or suspected, to express the SGA-1M gene, such as breast cancer cells, or other types of cancer cells, including metastases, may be isolated and tested utilizing hybridization or PCR techniques as described, above. The isolated cells can be derived from cell culture or from a patient. The analysis of cells taken from culture may be a necessary step in the assessment of cells to be used as part of a cell-based gene therapy technique or, alternatively, to test the effect of compounds on the expression of the SGA-1M gene. Such analyses may reveal both quantitative and qualitative aspects of the expression pattern of the SGA-1M gene, including activation or inactivation of SGA-1M gene expression and presence of alternatively spliced SGA-1M transcripts.

In one embodiment of such a detection scheme, a cDNA molecule is synthesized from an RNA molecule of interest by reverse transcription. All or part of the resulting cDNA is then used as the template for a nucleic acid amplification reaction, such as a PCR or the like. The nucleic acid reagents used as synthesis initiation reagents (e.g., primers) in the reverse transcription and nucleic acid amplification steps of this method are chosen from among the SGA-1M gene nucleic acid reagents described in Section 5.1. The preferred lengths of such nucleic acid reagents are at least 9-30 nucleotides.

For detection of the amplified product, the nucleic acid amplification may be performed using radioactively or non-radioactively labeled nucleotides. Alternatively, enough amplified product may be made such that the product may be visualized by standard ethidium bromide staining or by utilizing any other suitable nucleic acid staining method.

RT-PCR techniques can be utilized to detect differences in SGA-1M transcript size which may be due to normal or abnormal alternative splicing. Additionally, such techniques can be performed using standard techniques to detect quantitative differences between levels of full length and/or alternatively spliced SGA-1M transcripts detected in normal individuals relative to those individuals having cancer or exhibiting a predisposition toward neoplastic changes.

In the case where detection of particular alternatively spliced species is desired, appropriate primers and/or hybridization probes can be used, such that, in the absence of such sequence, no amplification would occur. Alternatively, primer pairs may be chosen utilizing the sequence data depicted in FIG. 2 to choose primers which will yield fragments of differing size depending on whether a particular exon is present or absent from the transcript SGA-1M transcript being utilized.

As an alternative to amplification techniques, standard Northern analyses can be performed if a sufficient quantity of the appropriate cells can be obtained. The preferred length of a probe used in a Northern analysis is 9-50 nucleotides. Utilizing such techniques, quantitative as well as size related differences between SGA-1M transcripts can also be detected.

Additionally, it is possible to perform such SGA-1M gene expression assays in situ, i.e., directly upon tissue sections (fixed and/or frozen) of patient tissue obtained from biopsies or resections, such that no nucleic acid purification is necessary. Nucleic acid reagents such as those described in Section 5.1 may be used as probes and/or primers for such in situ procedures (see, e.g., Nuovo, G. J., 1992, PCR In Situ Hybridization: Protocols And Applications, Raven Press, N.Y.).

Mutations or polymorphisms within the SGA-1M gene can be detected by utilizing a number of techniques. Nucleic acid from any nucleated cell can be used as the starting point for such assay techniques, and may be isolated according to standard nucleic acid preparation procedures which are well known to those of skill in the art. For the detection of SGA-1M mutations, any nucleated cell can be used as a starting source for genomic nucleic acid. For the detection of,SGA-1M transcripts or SGA-1M gene products, any cell type or tissue in which the SGA-1M gene is expressed, such as, for example, breast cancer cells, including metastases, may be utilized.

Genomic DNA may be used in hybridization or amplification assays of biological samples to detect abnormalities involving SGA-1M gene structure, including point mutations, insertions, deletions and chromosomal rearrangements. Such assays may include, but are not limited to, direct sequencing (Wong, C. et al., 1987, Nature 330:384), single stranded conformational polymorphism analyses (SSCP; Orita, M. et al., 1989, Proc. Natl. Acad. Sci. USA 86:2766), heteroduplex analysis (Keen, T. J. el al., 1991, Genomics 11:199; Perry, D. J. & Carrell, R. W., 1992), denaturing gradient gel electrophoresis (DGGE; Myers, R. M. et al., 1985, Nucl. Acids Res. 13:3131), chemical mismatch cleavage (Cotton, R. G. et al., 1988, Proc. Natl. Acad Sci. USA 85:4397) and oligonucleotide hybridization (Wallace, R. B. et al., 1981, Nucl. Acids Res. 9:879; Lipshutz, R. J. et al., 1995, Biotechniques 19:442).

Diagnostic methods for the detection of SGA-1M nucleic acid molecules, in patient samples or other appropriate cell sources, may involve the amplification of specific gene sequences, e.g., by the polymerase chain reaction (PCR; See Mullis, K. B., 1987, U.S. Pat. No. 4,683,202), followed by the analysis of the amplified molecules using techniques well known to those of skill in the art, such as, for example, those listed above. Utilizing analysis techniques such as these, the amplified sequences can be compared to those which would be expected if the nucleic acid being amplified contained only normal copies of the SGA-1M gene in order to determine whether an SGA-1M gene mutation exists.

Further, well-known genotyping techniques can be performed to type polymorphisms that are in close proximity to mutations in the SGA-1M gene itself. These polymorphisms can be used to identify individuals in families likely to carry mutations. If a polymorphism exhibits linkage disequilibrium with mutations in the SGA-1M gene, it can also be used to identify individuals in the general population likely to carry mutations. Polymorphisms that can be used in this way include restriction fragment length polymorphisms (RFLPs), which involve sequence variations in restriction enzyme target sequences, single-base polymorphisms and simple sequence repeat polymorphisms (SSLPs).

For example, Weber (U.S. Pat. No. 5,075,217) describes a DNA marker based on length polymorphisms in blocks of (dC-dA)n-(dG-dT)n short tandem repeats. The average separation of (dC-dA)n-(dG-dT)n blocks is estimated to be 30,000-60,000 bp. Markers which are so closely spaced exhibit a high frequency co-inheritance, and are extremely useful in the identification of genetic mutations, such as, for example, mutations within the SGA-1M gene, and the diagnosis of diseases and disorders related to SGA-1M mutations.

Also, Caskey et al. (U.S. Pat. No. 5,364,759), describe a DNA profiling assay for detecting short tri and tetra nucleotide repeat sequences. The process includes extracting the DNA of interest, such as the SGA-1M gene, amplifying the extracted DNA, and labeling the repeat sequences to form a genotypic map of the individual's DNA.

An SGA-1M probe could be used to directly identify RFLPs. Additionally, an SGA-1M probe or primers derived from the SGA-1M sequence could be used to isolate genomic clones such as YACs, BACs, PACs, cosmids, phage or plasmids. The DNA contained in these clones can be screened for single-base polymorphisms or simple sequence length polymorphisms (SSLPs) using standard hybridization or sequencing procedures.

Alternative diagnostic methods for the detection of SGA-1M gene expression, SGA-1M gene mutations or polymorphisms can include hybridization techniques which involve for example, contacting and incubating nucleic acids including recombinant DNA molecules, cloned genes or degenerate variants thereof, obtained from a sample, e.g., derived from a patient sample or other appropriate cellular source, with one or more labeled nucleic acid reagents including recombinant DNA molecules, cloned genes or degenerate variants thereof, as described in Section 5.1, under conditions favorable for the specific or selective annealing of these reagents to their complementary sequences within the SGA-1M gene. Preferably, the lengths of these nucleic acid reagents are at least 9 to 50 nucleotides. After incubation, all non-annealed nucleic acids are removed from the nucleic acid:SGA-1M molecule hybrid. The presence of nucleic acids which have hybridized, if any such molecules exist, is then detected. Using such a detection scheme, the nucleic acid from the cell type or tissue of interest can be immobilized, for example, to a solid support such as a membrane, or a plastic surface such as that on a microtiter plate or polystyrene beads. In this case, after incubation, non-annealed, labeled nucleic acid reagents of the type described in Section 5.1 are easily removed. Detection of the remaining, annealed, labeled SGA-1M nucleic acid reagents is accomplished using standard techniques well-known to those in the art. The SGA-1M gene sequences to which the nucleic acid reagents have annealed can be compared to the annealing pattern expected from a normal SGA-1 M gene sequence in order to determine whether an SGA-1M gene mutation is present.

### 5.4.2. Detection of SGA-1M-Encoded Proteins

Detection of the SGA-1M gene product includes the detection of the proteins encoded for by SEQ ID NO:2 and/or SEQ ID NO:4. Detection of elevated levels of SGA-1M(A) and/or SGA-1M(B), compared to a non-cancerous sample or a non-cancerous predetermined standard can indicate the presence of, or predisposition to developing cancer in a subject. Detection of elevated levels of said protein, in a subject compared to a non-cancerous sample or a non-cancerous predetermined standard can indicate the likelihood of metastasis of a cancer in the subject, and/or poor prognosis for the subject. The diagnosis and/or prognosis of cancer involves the detection of naturally occurring SGA-1M polypeptides in a subject. Detection of an SGA-1M polypeptide can be by any method known in the art.

Antibodies directed against naturally occurring SGA-1M(A), SGA-1M(B), or naturally occurring variants thereof or peptide fragments thereof, which are discussed, above, in Section 5.2, may be used as diagnostics and prognostics, as described herein. Such diagnostic methods, may be used to detect abnormalities in the level of SGA-1M gene expression, or abnormalities in the structure and/or temporal, tissue, cellular, or subcellular location of the SGA-1M-encoded polypeptide. Antibodies, or fragments of antibodies, such as those described below, may be used to screen potentially therapeutic compounds in vitro to determine their effects on SGA-1M gene expression and SGA-1M-encoded polypeptide production. The compounds which have beneficial effects on cancer, e.g., breast cancer can be identified and a therapeutically effective dose determined.

The tissue or cell type to be analyzed will generally include those which are known, or suspected, to express the SGA-1M gene, such as, for example, cancer cells including breast cancer cells, ovarian cancer cells, skin cancer cells, lymphoid cancer cells, and metastatic forms thereof. The protein isolation methods employed herein may, for example, be such as those described in Harlow and Lane (Harlow, E. and Lane, D., 1988, Antibodies: A Laboratory Manual, Cold Spring Harbor Laboratory Press, Cold Spring Harbor, N.Y.). The isolated cells can be derived from cell culture or from a patient. The analysis of cells taken from culture may be a necessary step to test the effect of compounds on the expression of the SGA-1M gene.

Preferred diagnostic methods for the detection of SGA-1M gene products or conserved variants or peptide fragments thereof, may involve, for example, immunoassays wherein the SGA-1M gene products or conserved variants, including gene products which are the result of alternatively spliced transcripts, or peptide fragments are detected by their interaction with an anti-SGA-1M gene product-specific or -selective antibody.

For example, antibodies, or fragments of antibodies, such as those described above in Section 5.3, useful in the present invention may be used to quantitatively or qualitatively detect the presence of SGA-1M-encoded polypeptides or naturally occurring variants or peptide fragments thereof. The antibodies (or fragments thereof) useful in the present invention may, additionally, be employed histologically, as in immunofluorescence or immunoelectron microscopy, for in situ detection of SGA-1M gene products or conserved variants or peptide fragments thereof In situ detection may be accomplished by removing a histological specimen from a subject, such as paraffin embedded sections of tissue, e.g., breast tissues and applying thereto a labeled antibody of the present invention. The antibody (or fragment) is preferably applied by overlaying the labeled antibody (or fragment) onto a biological sample. Since the SGA-1M gene product is present in the cytoplasm, it may be desirable to introduce the antibody inside the cell, for example, by making the cell membrane permeable. The SGA-1M polypeptides may also be expressed on the cell surface, thus cells can be directly labeled by applying antibodies that are specific or selective for the SGA-1M polypeptides or fragment thereof to the cell surface.

Through the use of such a procedure, it is possible to determine not only the presence of the SGA-1M gene product, or naturally occurring variants thereof or peptide fragments, but also its distribution in the examined tissue. Using the methods of the present invention, those of ordinary skill will readily perceive that any of a wide variety of histological methods (such as staining procedures) can be modified in order to achieve such in situ detection.

Immunoassays for SGA-1M-encoded polypeptides or conserved variants or peptide fragments thereof will typically comprise contacting a sample, such as a biological fluid, tissue or a tissue extract, freshly harvested cells, or lysates of cells which have been incubated in cell culture, in the presence of an antibody that specifically or selectively binds to an SGA-1M gene product, e.g., a detectably labeled antibody capable of identifying SGA-1M polypeptides or conserved variants or peptide fragments thereof, and detecting the bound antibody by any of a number of techniques well-known in the art (e.g., Western blot, ELISA, FACS).

The biological sample may be brought in contact with and immobilized onto a solid phase support or carrier such as nitrocellulose, or other solid support which is capable of immobilizing cells, cell particles or soluble proteins. The support may then be washed with suitable buffers followed by treatment with the detectably labeled antibody that selectively or specifically binds to an SGA-1M-encoded polypeptide. The solid phase support may then be washed with the buffer a second time to remove unbound antibody. The amount of bound label on solid support may then be detected by conventional means.

By “solid phase support or carrier” is intended any support capable of binding an antigen or an antibody. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present invention. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet, test strip, etc. Preferred supports include polystyrene beads. Those skilled in the art will know many other suitable carriers for binding antibody or antigen, or will be able to ascertain the same by use of routine experimentation.

The anti-SGA-1M(A) or anti-SGA-1M(B) antibody can be detectably labeled by linking the same to an enzyme and using the labeled antibody in an enzyme immunoassay (EIA) (Voller, A., “The Enzyme Linked Immunosorbent Assay (ELISA)”, 1978, Diagnostic Horizons 2:1, Microbiological Associates Quarterly Publication, Walkersville, Md.); Voller, A. et al, 1978, J. Clin. Pathol. 31:507-520; Butler, J. E., 1981, Meth. Enzymol. 73:482; Maggio, E. (ed.), 1980, Enzyme Immunoassay, CRC Press, Boca Raton, Fla.,; Ishikawa, E. et al., (eds.), 1981, Enzyme Immunoassay, Kgaku Shoin, Tokyo). The enzyme which is bound to the antibody will react with an appropriate substrate, preferably a chromogenic substrate, in such a manner as to produce a chemical moiety which can be detected, for example, by spectrophotometric, fluorimetric or by visual means. Enzymes which can be used to detectably label the antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoarnylase and acetylcholinesterase. The detection can be accomplished by colorimetric methods which employ a chromogenic substrate for the enzyme. Detection may also be accomplished by visual comparison of the extent of enzymatic reaction of a substrate in comparison with similarly prepared standards.

Detection may also be accomplished using any of a variety of other immunoassays. For example, by radioactively labeling the antibodies or antibody fragments, it is possible to detect SGA-1M-encoded polyepeptides through the use of a radioimmunoassay (RIA) (see, for example, Weintraub, B., Principles of Radioimmunoassays, Seventh Training Course on Radioligand Assay Techniques, The Endocrine Society, March, 1986). The radioactive isotope can be detected by such means as the use of a gamma counter or a scintillation counter or by autoradiography.

It is also possible to label the antibody with a fluorescent compound. When the fluorescently labeled antibody is exposed to light of the proper wave length, its presence can then be detected due to fluorescence. Among the most commonly used fluorescent labeling compounds are fluorescein isothiocyanate, rhodamine, pbycoerythrin, phycocyanin, allophycocyanin, o-phthaldehyde and fluorescamine.

The antibody can also be detectably labeled using fluorescence emitting metals such as ¹⁵²Eu, or others of the lanthanide series. These metals can be attached to the antibody using such metal chelating groups as diethylenetriaminepentacetic acid (DTPA) or ethylenediaminetetraacetic acid (EDTA).

The antibody also can be detectably labeled by coupling it to a chemiluminescent compound. The presence of the chemiluminescent-tagged antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

Likewise, a bioluminescent compound may be used to label the antibody of the present invention. Bioluminescence is a type of chemiluminescence found in biological systems in, which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

In various embodiments, the present invention provides methods for the measurement of SGA-1M polyepeptides, and the uses of such measurements in clinical applications using SGA-1M(A) or SGA-1M(B)-specific or -selective antibodies.

The measurement of SGA-1M polypepeptides of the invention can be valuable in detecting and/or staging breast cancer and other cancers (e.g., ovarian cancer, skin cancer (e.g., melanoma), a cancer of the lymphoid system (e.g., lymphoma), thyroid cancer (e.g., thyroid carcinoma), pancreatic cancer (e.g., pancreas adenocarcinoma), stomach cancer (e.g., stomach adenocarcinoma), or lung cancer (e.g., lung adenocarcinoma)) in a subject, in screening of these and other cancers in a population, in differential diagnosis of the physiological condition of a subject, and in monitoring the effect of a therapeutic treatment on a subject.

The present invention also provides for the detecting, diagnosing, or staging of breast cancer and other cancers (e.g., ovarian cancer, skin cancer (e.g., melanoma), a cancer of the lymphoid system (e.g., lymphoma), thyroid cancer (e.g., thyroid carcinoma), pancreatic cancer (e.g., pancreas adenocarcinoma), stomach cancer (e.g., stomach adenocarcinoma), or lung cancer (e.g., lung adenocarcinoma)), or the monitoring of treatment of such cancers by measuring the level of expression of the SGA-1M polypeptide. In addition to the SGA-1M polypeptide at least one other marker,;such as receptors or differentiation antigens can also be measured. For example, serum markers selected from, for example but not limited to, carcinoembryonic antigen (CEA), CA15-3, CA549, CAM26, M29, CA27.29 and MCA can be measured in combination with the SGA-1M polypeptide to detect, diagnose, stage, or monitor treatment of breast cancer and other cancers. In another embodiment, the prognostic indicator is the observed change in different marker levels relative to one another, rather than the absolute levels of the markers present at any one time. These measurements can also aid in predicting therapeutic outcome and in evaluating and monitoring the overall disease status of a subject.

In a specific embodiment of the invention, soluble SGA-1M polypeptide alone or in combination with other markers can be measured in any body fluid of the subject including but not limited to blood, serum, plasma, milk, urine, saliva, pleural efflusions, synovial fluid, spinal fluid, tissue infiltrations and tumor infiltrates. In another embodiment the SGA-1M polypeptide is measured in tissue samples or cells directly. The present invention also contemplates a kit for measuring the level of SGA-1M expression in a biological sample and the use of said kit to diagnose a subject with cancer. Alternatively said kit could be used to determine the prognosis of a cancer patient or the risk of metastasis of said cancer.

Any of numerous immunoassays can be used in the practice of the methods of the instant invention, such as those described in Section 5.4.2. Antibodies, or antibody fragments containing the binding domain, which can be employed include but are not limited to suitable antibodies among those in Section 5.3 and other antibodies known in the art or which can be obtained by procedures standard in the art such as those described in Section 5.3.

### 5.4.2.1 In Vivo Imaging Using Antibodies to an SGA-1M Polypeptide

Current diagnostic and therapeutic methods make use of antibodies to target imaging agents or therapeutic substances, e.g., to tumors. Thus, labeled antibodies specific or selective for an SGA-1M polypepeptide can be used in the methods of the invention for the in vivo imaging, detection, and treatment of cancer in a subject.

Antibodies may be linked to chelators such as those described in U.S. Pat. No. 4,741,900 or U.S. Pat. No. 5,326,856. The antibody-chelator complex may then be radiolabeled to provide an imaging agent for diagnosis or treatment of disease. The antibodies may also be used in the methods that are disclosed in U.S. Pat. No. 5,449,761 for creating a radiolabeled antibody for use in imaging or radiotherapy.

In in vivo diagnostic applications, specific tissues or even specific cellular disorders, e.g., cancer, may be imaged by administration of a sufficient amount of a labeled antibodies using the methods of the instant invention.

A wide variety of metal ions suitable for in vivo tissue imaging have been tested and utilized clinically. For imaging with radioisotopes, the following characteristics are generally desirable: (a) low radiation dose to the patient; (b) high photon yield which permits a nuclear medicine procedure to be performed in a short time period; (c) ability to be produced in sufficient quantities; (d) acceptable cost; (e) simple preparation for administration; and (f) no requirement that the patient be sequestered subsequently. These characteristics generally translate into the following: (a) the radiation exposure to the most critical organ is less than 5 rad; (b) a single image can be obtained within several hours after infusion; (c) the radioisotope does not decay by emission of a particle; (d) the isotope can be readily detected; and (e) the half-life is less than four days (Lamb and Kramer, “Commercial Production of Radioisotopes for Nuclear Medicine”, In Radiotracers For Medical Applications, Vol. 1, Rayudu (Ed.), CRC Press, Inc., Boca Raton, pp. 17-62). Preferably, the metal is technetium-99m.

By way of illustration, the targets that one may image include any solid neoplasm, certain organs such a lymph nodes, parathyroids, spleen and kidney, sites of inflammation or infection (e.g., macrophages at such sites), myocardial infarction or thromboses (neoantigenic determinants on fibrin or platelets), and the like evident to one of ordinary skill in the art. Furthermore, the neoplastic tissue may be present in bone, internal organs, connective tissue, or skin.

As is also apparent to one of ordinary skill in the art, one may use the methods of the present invention in in vivo therapeutics (e.g., using radiotherapeutic metal complexes), especially after having diagnosed a diseased condition via the in vivo diagnostic method described above, or in in vitro diagnostic application (e.g., using a radiometal or a fluorescent metal complex).

Accordingly, a method of diagnosing cancer by obtaining an image of an internal region of a subject is contemplated in the instant invention which comprises administering to a subject an effective amount of an antibody composition specific or selective for an SGA-1M polypeptide conjugated with a metal in which the metal is radioactive, and recording the scintigraphic image obtained from the decay of the radioactive metal. Likewise, a method is contemplated of enhancing a magnetic resonance (MR) image of an internal region of a subject which comprises administering to a subject an effective amount of an antibody composition containing a metal in which the metal is paramagnetic, and recording the MR image of an internal region of the subject.

Other methods include a method of enhancing a sonographic image of an internal region of a subject comprising administering to a subject an effective amount of an antibody composition containing a metal and recording the sonographic image of an internal region of the subject. In this latter application, the metal is preferably any non-toxic heavy metal ion. A method of enhancing an X-ray image of an internal region of a subject is also provided -which comprises administering to a subject an antibody composition containing a metal, and recording the X-ray image of an internal region of the subject. A radioactive, non-toxic heavy metal ion is preferred.

### 5.4.3. Detecting and Staging Cancer in a Subject

The methods of the present invention include measurement of naturally occurring SGA-1M polypeptide, or naturally occurring variants thereof, or fragment thereof, soluble SGA-1M polypeptide or intra-cellular SGA-1M polypeptides to detect breast cancer, ovarian cancer, skin cancer (e.g., melanoma), a cancer of the lymphoid system (e.g., lymphoma), thyroid cancer (e.g., thyroid carcinoma), pancreatic cancer (e.g., pancreas adenocarcinoma), stomach cancer (e.g., stomach adenocarcinoma), or lung cancer (e.g., lung adenocarcinoma)) or other cancers in a subject or to stage such cancers in a subject.

Staging refers to the grouping of patients according to the extent of their disease. Staging is useful in choosing treatment for individual patients, estimating prognosis, and comparing the results of different treatment programs. Staging of breast cancer for example is performed initially on a clinical basis, according to the physical examination and laboratory radiologic evaluation. The most widely used clinical staging system is the one adopted by the International Union against Cancer (UICC) and the American Joint Committee on Cancer (AJCC) Staging and End Results Reporting. It is based on the tumor-nodes-metastases (TNM) system as detailed in the 1988 Manual for Staging of Cancer. Breast cancer diseases or conditions which may be detected and/or staged in a subject according to the present invention include but are not limited to those listed in Table 2.

TABLE 2
STAGING OF BREAST CANCER

| T | PRIMARY TUMORS | |
|---|---|---|
| TX | Primary tumor cannot be assessed | |
| T0 | No evidence of primary tumor | |
| Tis | Carcinoma in situ: intraductal carcinoma, lobular carcinoma, | |
| | or Paget's disease with no tumor | |
| T1 | Tumor 2 cm or less in its greatest dimension | |
| | a. | 0.5 cm or less in greatest dimension |
| | b. | Larger than 0.5 cm, but not larger than 1 cm in greatest |
| | | dimension |
| | c. | Larger than 1 cm, but not larger than 2 cm in greatest |
| | | dimension |
| T2 | Tumor more than 2 cm but not more than 5 cm in greatest | |
| | dimension | |
| T3 | Tumor more than 5 cm in its greatest dimension | |
| T4 | Tumor of any size with direct extension to chest wall or | |
| | to skin. Chest wall includes ribs, intercostal muscles, | |
| | and serratus anterior muscle, but not pectoral muscle. | |
| | a. | Extension to chest wall |
| | b. | Edema (including peau d'orange), ulceration of the skin |
| | | of the breast, or satellite skin nodules confined to |
| | | the same breast |
| | c. | Both of the above |
| | d. | Inflammatory carcinoma |

Dimpling of the skin, nipple retraction, or any other skin changes
except those in T4b may occur in T1, T2 or T3 without affecting
the classification.

| | |
|---|---|
| N | REGIONAL LYMPH NODES |
| NX | Regional lymph nodes cannot be (e.g., previously removed) |
| N0 | No regional lymph node metastases |
| N1 | Metastasis to movable ipsilateral axillary node(s) |
| N2 | Metastases to ipsilateral axillary nodes fixed to one another |
| | or to other structures |
| N3 | Metastases to ipsilateral internal mammary lymph node(s) |
| M | DISTANT METASTASIS |
| M0 | No evidence of distant metastasis |
| M1 | Distant metastases (including metastases to ipsilateral |
| | supraclavicular lymph nodes) |

Methods of staging of cancers other than breast cancer (e.g., ovarian cancer, skin cancer (e.g., melanoma), a cancer of the lymphoid system (e.g., lymphoma), thyroid cancer (e.g., thyroid carcinoma), pancreatic cancer (e.g., pancreas adenocarcinoma), stomach cancer (e.g., stomach adenocarcinoma), or lung cancer (e.g., lung adenocarcinoma)) are well known to the skilled artisan and can be used in the methods of the present invention.

Any immunoassay, such as those described in Section 5.4.2 can be used to measure the amount of SGA-1M polypeptide or soluble SGA-1M polypeptide which is compared to a baseline level. This baseline level can be the amount which is established to be normally present in the tissue or body fluid of subjects with various degrees of the disease or disorder. An amount present in the tissue or body fluid of the subject which is similar to a standard amount, established to be normally present in the tissue or body fluid of the subject during a specific stage of cancer or breast cancer, is indicative of the stage of the disease in the subject. The baseline level could also be the level present in the subject prior to the onset of disease or the amount present during remission of the disease.

In specific embodiments of this aspect of the invention, measurements of levels of the SGA-1M polypeptide or soluble SGA-1M polypeptide can be used in the detection of infiltrative ductal carcinoma (IDC) or the presence of metastases or both. Increased levels of SGA-1M polypeptides or soluble SGA-1M polypeptide are associated with metastases.

In another embodiment of the invention, the measurement of soluble SGA-1M polypeptide, intra-cellular SGA-1M polypeptide, fragments thereof or immunologically related molecules can be used to differentially diagnose in a subject a particular disease phenotype or physiological condition as distinct as from among two or more phenotypes or physiological conditions. For example, measurements of SGA-1M polypeptide or soluble SGA-1M polypeptide levels may be used in the differential diagnosis of infiltrative ductal carcinoma, as distinguished from ductal carcinoma in situ or benign fibroadenomas. To this end, for example, the measured amount of the SGA-1M polypeptide is compared with the amount of the molecule normally present in the tissue, cells or body fluid of a subject with one of the suspected physiological conditions. A measured amount of the SGA-1M polypeptide similar to the amount normally present in a subject with one of the physiological conditions, and not normally present in a subject with one or more of the other physiological conditions, is indicative of the physiological condition of the subject.

As an alternative to measuring levels of SGA-1M polypeptides in the foregoing staging methods, levels of SGA-1M transcript can be measured, for example by the methods described in Section 5.4.1, supra.

### 5.4.4. Monitoring the Effect of a Therapeutic Treatment

The present invention provides a method for monitoring the effect of a therapeutic treatment on a subject who has undergone the therapeutic treatment.

Clinicians very much need a procedure that can be used to monitor the efficacy of cancer treatments. SGA-1M-encoded polypeptides and/or transcripts can be identified and detected in breast cancer patients or other cancer patients with different manifestations of disease, providing a sensitive assay to monitor therapy. The therapeutic treatments which may be evaluated according to the present invention include but are not limited to radiotherapy, surgery, chemotherapy, vaccine administration, endocrine therapy, immunotherapy, and gene therapy, etc. The chemotherapeutic regimens include, but are not limited to administration of drugs such as, for example, methotrexate, fluorouracil, cyclophosphamide, doxorubicin, and taxol. The endocrine therapeutic regimens include, but are not limited to administration of tamoxifen, progestins, etc.

The method of the invention comprises measuring at suitable time intervals before, during, or after therapy, the amount of an SGA-1M transcript or polypeptide (including soluble polypeptide), or any combination of the foregoing. Any change or absence of change in the absolute or relative amounts of the SGA-1M gene products can be identified and correlated with the effect of the treatment on the subject.

In particular, the serum- or cell- associated levels of an SGA-1M-encoded polypeptide can bear a direct relationship with severity of breast cancer, or other cancer, risk of metastasis of said cancer and poor prognosis. Since serum- or cell-associated SGA-1M polypeptide levels are generally undetectable or negligible in normal individuals, generally, a decrease in the level of detectable SGA-1M polypeptide after a therapeutic treatment is associated with efficacious treatment.

In a preferred aspect, the approach that can be taken is to determine the levels of soluble or cell associated SGA-1M polyepeptide levels at different time points and to compare these values with a baseline level. The baseline level can be either the level of the SGA-1M polypeptide present in normal, disease free individuals; and/or the levels present prior to treatment, or during remission of disease, or during periods of stability. These levels can then be correlated with the disease course or treatment outcome.

### 5.4.5. Prognostic Assays

The methods described herein can furthermore be utilized as prognostic assays to identify subjects having or at risk of developing cancer or another disease or disorder associated with aberrant expression or activity of an SGA-1M polypeptide. For example, the assays described herein, such as the preceding diagnostic assays or the following assays, can be utilized to identify a subject having or at risk of developing cancer, e.g., breast cancer, ovarian cancer, skin cancer, a cancer of the lymphoid system, thyroid cancer, pancreatic cancer, stomach cancer, or lung cancer, or another disorder associated with aberrant expression or activity of an SGA-1M polypeptide. Thus, the present invention provides a method in which a test sample is obtained from a subject and an SGA-1M polypeptide or nucleic acid (e.g., mRNA, genomic DNA) of the invention is detected, wherein the presence of the polypeptide or nucleic acid is diagnostic for a subject having or at risk of developing a disease or disorder associated with aberrant expression or activity of the SGA-1M polypeptide, e.g., cancer. As used herein, a “test sample” refers to a biological sample obtained from a subject of interest. For example, a test sample can be a biological fluid (e.g., serum), cell sample, or tissue.

The prognostic assays described herein, for example, can be used to identify a subject having or at risk of developing disorders such as cancers, for example, hormone-sensitive cancer such as breast cancer.

In another example, prognostic assays described herein can be used to identify a subject having or at risk of developing related disorders associated with expression of polypeptides or nucleic acids of the invention.

Furthermnore, the prognostic assays described herein can be used to determine whether a subject can be administered an agent (e.g., an agonist, antagonist, peptidomimetic, protein, peptide, nucleic acid, small molecule, or other drug candidate) to treat cancer or another disease or disorder associated with aberrant expression or activity of an SGA-1M polypeptide. For example, such methods can be used to determine whether a subject can be effectively treated with a specific agent or class of agents (e.g., agents of a type which decrease activity or expression level of an SGA-1M transcript or polypeptide). Thus, the present invention provides methods for determining whether a subject can be effectively treated with an agent for a disorder associated with aberrant expression or activity of the SGA-1M transcript or polypeptide in which a test sample is obtained and the polypeptide or nucleic acid encoding the polypeptide is detected (e.g., wherein the presence of the polypeptide or nucleic acid is diagnostic for a subject that can be administered the agent to treat a disorder associated with aberrant expression or activity of the SGA-1M transcript or polypeptide).

The methods of the invention can also be used to detect genetic lesions or mutations in an SGA-1M gene, thereby determining if a subject with the lesioned gene is at increased or reduced risk for a disorder characterized by aberrant expression or activity of a polypeptide of the invention, e.g., cancer. In one embodiment, the methods include detecting, in a sample of cells from the subject, the presence or absence of a genetic lesion or mutation characterized by at least one of an alteration affecting the integrity of a gene encoding an SGA-1M polypeptide, or the mis-expression of the gene encoding an SGA-1M polypeptide. For example, such genetic lesions or mutations can be detected by ascertaining the existence of at least one of: 1) a deletion of one or more nucleotides from an SGA-1M gene; 2) an addition of one or more nucleotides to an SGA-1M gene; 3) a substitution of one or more nucleotides of an SGA-1M gene i.e. a point mutation; 4) a chromosomal rearrangement of an SGA-1M gene; 5) an alteration in the level of a messenger RNA transcript of an SGA-1M gene; 6) an aberrant modification of an SGA-1M gene, such as of the methylation pattern of the genomic DNA; 7) the presence of a non-wild type splicing pattern of a messenger RNA transcript of an SGA-1M gene; 8) a non-wild type level of the protein encoded by an SGA-1M gene; 9) an allelic loss of an SGA-1M gene; and 10) an inappropriate post-translational modification of a protein encoded by an SGA-1M gene. As described herein, there are a large number of assay techniques known in the art which can be used for detecting lesions in a gene.

In certain embodiments, methods for the detection of the lesion involves the use of a probe/primer in a polymerase chain reaction (PCR) (See, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077; and Nakazawa et al. (1994) Proc Natl Acad Sci. USA 91:360), the latter of which can be particularly useful for detecting point mutations in a gene (see, e.g., Abravaya el al. (1995) Nucleic Acids Res. 23:675). These methods are useful in the diagnosis and prognosis of cancer in a subject. This method can include the steps of collecting a sample of cells from a patient, isolating nucleic acid (e.g., genomic, mRNA or both) from the cells of the sample, contacting the nucleic acid sample with one or more primers which specifically hybridize to the selected gene under conditions such that hybridization and amplification of the gene or gene product (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Mutations in a selected gene from a sample cell or tissue can also be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,498,531) can be used to score for the presence of specific mutations by development or loss of a ribozyme cleavage site.

In other embodiments, methods are provided whereby genetic mutations can be identified by hybridizing a sample and control nucleic acids, e.g., DNA or RNA, to high density arrays comprising hundreds or thousands of oligonucleotides probes (Cronin et al. 1996, Human Mutation 7:244; Kozal et al. 1996, Nature Medicine 2:753). For example, genetic mutations can be identified in two-dimensional arrays containing light-generated DNA probes as described in Cronin et al., supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

Sequencing reactions known in the art can be used to directly sequence the selected gene and detect mutations in the SGA-1M gene by comparing the sequence of the sample nucleic acids with the corresponding wild-type (control) sequence. Examples of sequencing reactions include those based on techniques developed by Maxim and Gilbert (Maxim and Gilbert, 1977, Proc Natl Acad Sci. USA 74:560) or Sanger (Sanger et al. 1977, Proc Natl Acad Sci. USA 74:5463). Such methods are useful in the diagnosis and prognosis of a subject with cancer. It is also contemplated that any of a variety of automated sequencing procedures can be utilized when performing the diagnostic assays (Naeve et al., 1995, BioTechniques 19:448), including sequencing by mass spectrometry (see, e.g., PCT Publication No. WO 94/16101; Cohen et al. 1996, Adv. Chromatogr. 36:127; and Griffin et al., 1993, Appl. Biochem. Biotechnol. 38:147).

Furthermore, the presence of an SGA-1M nucleic acid molecule or polypeptide of the invention can be correlated with the presence or expression level of other cancer-related proteins, such as for example, androgen receptor, estrogen receptor, adhesion molecules (e.g., E-cadherin), proliferation markers (e.g., MIB-1), tumor-suppressor genes (e.g., TP53, retinoblastoma gene product), vascular endothelial growth factor (Lissoni et al., 2000, Int J Biol Markers. 15(4):308), Rad51 (Maacke et al., 2000, Int J Cancer. 88(6):907), cyclin D1, BRCA1, BRCA2, or carcinoembryonic antigen.

The methods described herein may be performed, for example, by utilizing pre-packaged diagnostic kits comprising at least one nucleic acid probe or antibody reagent described herein, which may be conveniently used, e.g., in clinical settings to diagnose patients exhibiting symptoms or family history of a disease or illness involving a gene encoding a polypeptide of the invention. Furthermore, any cell type or tissue, e.g., cancerous breast cells or tissue, in which the SGA-1M gene is expressed may be utilized in the prognostic assays described herein.

### 5.5. Screening Assays for Compounds that Modulate SGA-1M Activity

The present invention further provides methods for the identification of compounds that may, through their interaction with the SGA-1M gene or SGA-1M gene product, affect the onset, progression and metastatic spread of breast cancer, ovarian cancer, skin cancer, a cancer of the lymphoid system, thyroid cancer, pancreatic cancer, stomach cancer, or lung cancer.

The following assays are designed to identify: (i) compounds that bind to SGA-1M gene products; (ii) compounds that bind to other proteins that interact with an SGA-1M gene product; (iii) compounds that interfere with the interaction of the SGA-1M gene product with other proteins; (iv) compounds that modulate the activity of an SGA-1M gene (i.e., modulate the level of SGA-1M gene expression, including transcription of the SGA-1M gene and/or translation of its encoded transcript, and/or modulate the level of SGA-1M-encoded polyepeptide activity); and (iv) compounds that modulate the activity of an SGA-1M gene product (e.g., modulate the acitvity of an SGA-1M-encoded polypeptide).

Assays may additionally be utilized which identify compounds which bind to SGA-1M gene regulatory sequences (e.g., promoter sequences), which may modulate the level of SGA-1M gene expression (see e.g., Platt, K. A., 1994, J. Biol. Chem. 269:28558).

Such proteins that interact with SGA-1M may be involved in the onset, development and metastatic spread of breast cancer, ovarian cancer, skin cancer, a cancer of the lymphoid system, thyroid cancer, pancreatic cancer, stomach cancer, or lung cancer, or other cancers.

The present invention also provides methods of using isolated SGA-1M nucleic acid molecules, or derivatives thereof, as probes that can be used to screen for DNA-binding proteins, including but not limited to proteins that affect DNA conformation or modulate transcriptional activity (e.g., enhancers, transcription factors). In another embodiment, such probes can be used to screen for RNA-binding factors, including but not limited to proteins, steroid hormones, or other small molecules. In yet another embodiment, such probes can be used to detect and identify molecules that bind or affect the pharmacokinetics or activity (e.g., enzymatic activity) of the SGA-1M gene or gene product. The proteins or nucleic acid binding factors or transcriptional modulators identified by a screening assay would provide an appropriate target for anti-cancer therapeutics.

In one embodiment, a screening assay of the invention can identify a test compound that is useful for increasing or decreasing the translation of one or both SGA-1M ORFs, for example, by binding to one or more regulatory elements in the 5′ untranslated region, the 3′ untranslated region, or the coding regions of the MRNA. Compounds that bind to mRNA can, inter alia, increase or decrease the rate of mRNA processing, alter its transport through the cell, prevent or enhance binding of the mRNA to ribosomes, suppressor proteins or enhancer proteins, or alter mRNA stability. Accordingly, compounds that increase or decrease mRNA translation can be used to treat or prevent disease. For example, diseases such as cancer, associated with overproduction of proteins, such as SGA-1M(A) or SGA-1M(B), can be treated or prevented by decreasing translation of the mRNA that codes for the overproduced protein, thus inhibiting production of the protein.

Accordingly, in one embodiment, a compound identified by a screening assay of the invention inhibits the production of an SGA-1M protein. In a further embodiment, the compound inhibits the translation of an SGA-1M mRNA. In yet another embodiment, the compound inhibits transcription of the SGA-1M gene.

The invention provides a method for identifying modulators, ie., candidate or test compounds or agents (e.g., peptides, peptidomimetics, small molecules or other drugs) which bind to the SGA-1M gene product or fragments thereof or have a stimulatory or inhibitory effect on, for example, expression or activity of the SGA-1M gene product or fragments thereof.

Compounds identified via assays such as those described herein may be useful, for example, in elaborating the biological function of the SGA-1M gene product, and for ameliorating symptoms of e.g., breast cancer, ovarian cancer, skin cancer, a cancer of the lymphoid system, thyroid cancer, pancreatic cancer, stomach cancer, or lung cancer or other types of cancer. Assays for testing the effectiveness of compounds, identified by, for example, techniques such as those described in Section 5.5.1, are discussed, below, in Section 5.5.3. It is to be noted that the compositions of the invention include pharmaceutical compositions comprising one or more of the compounds identified via such methods. Such pharmaceutical compositions can be formulated, for example, as discussed, below, in Section 5.7.

### 5.5.1. In Vitro Screening Assays for Compounds that Bind to the SGA-1M Gene Product

In vitro systems may be designed to identify compounds capable of interacting with, e.g., binding to, the SGA-1M gene product of the invention. Compounds identified may be useful, for example, in modulating the activity of wild type and/or mutant SGA-1M gene products, may be useful in elaborating the biological function of the SGA-1M gene product, may be utilized in screens for identifying compounds that disrupt normal SGA-1M gene product interactions, or may in themselves disrupt such interactions. Thus said compounds would be useful in treating, preventing and diagnosing cancer. In a particular embodiment said compounds are useful in the treatment, prevention and diagnosis of breast cancer, ovarian cancer, skin cancer, a cancer of the lymphoid system, thyroid cancer, pancreatic cancer, stomach cancer, or lung cancer.

The principle of the assays used to identify compounds that interact with the SGA-1M gene product involves preparing a reaction mixture of the SGA-1M gene product and the test compound under conditions and for a time sufficient to allow the two components to interact with, e.g., bind to, thus forming a complex, which can represent a transient complex, which can be removed and/or detected in the reaction mixture. These assays can be conducted in a variety of ways. For example, one method to conduct such an assay would involve anchoring SGA-1M gene product or the test substance onto a solid phase and detecting SGA-1M gene product/test compound complexes anchored on the solid phase at the end of the reaction. In one embodiment of such a method, the SGA-1M gene product may be anchored onto a solid surface, and the test compound, which is not anchored, may be labeled, either directly or indirectly.

In practice, microtiter plates may conveniently be utilized as the solid phase. The anchored component may be immobilized by non-covalent or covalent attachments. Non-covalent attachment may be accomplished by simply coating the solid surface with a solution of the protein and drying. Alternatively, an immobilized antibody, preferably a monoclonal antibody, specific or selective for the protein to be immobilized may be used to anchor the protein to the solid surface. The surfaces may be prepared in advance and stored.

In order to conduct the assay, the nonimmobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously nonimmobilized component is pre-labeled, the detection of label immobilized on the surface indicates that complexes were formed. Where the previously nonimmobilized component is not pre-labeled, an indirect label can be used to detect complexes anchored on the surface; e.g., using a labeled antibody specific or selective for the previously nonimmobilized component (the antibody, in turn, may be directly labeled or indirectly labeled with a labeled anti-Ig antibody).

Alternatively, a reaction can be conducted in a liquid phase, the reaction products separated from unreacted components, and complexes detected; e.g., using an immobilized antibody specific or selective for SGA-1M gene product or the test compound to anchor any complexes formed in solution, and a labeled antibody specific or selective for the other component of the possible complex to detect anchored complexes.

### 5.5.2 Assays for Proteins that Interact with the SGA-1M Gene Product

Any method suitable for detecting protein-protein interactions may be employed for identifying SGA-1M protein-protein interactions. Proteins that interact with SGA-1M will be potential therapeutics for the treatment of cancer. Thus the assays described below are useful in identifying proteins that can be used in methods to treat cancer. Proteins that interact with SGA-1M can also be used in the diagnosis of cancer. Thus the assays described below are also useful in methods to diagnose cancer.

Among the traditional methods which may be employed are co-immunoprecipitation, crosslinking and co-purification through gradients or chromatographic columns (e.g., size exclusion chromatography). Utilizing procedures such as these allows for the isolation of intracellular proteins which interact with SGA-1M gene products. Once isolated, such an intracellular protein can be identified and can, in turn, be used, in conjunction with standard techniques, to identify additional proteins with which it interacts. For example, at least a portion of the amino acid sequence of the intracellular protein which interacts with the SGA-1M gene product can be ascertained using techniques well known to those of skill in the art, such as via the Edman degradation technique (see, e.g., Creighton, 1983, Proteins: Structures and Molecular Principles, W.H. Freeman & Co., N.Y., pp. 34-49). The amino acid sequence obtained may be used as a guide for the generation of oligonucleotide mixtures that can be used to screen for gene sequences encoding such intracellular proteins. Screening may be accomplished, for example, by standard hybridization or PCR techniques. Techniques for the generation of oligonucleotide mixtures and the screening are well-known. (See, e.g., Ausubel, supra., and PCR Protocols: A Guide to Methods and Applications, 1990, Innis, M. et al., eds. Academic Press, Inc., New York).

Additionally, methods may be employed which result in the simultaneous identification of genes which encode a protein interacting with the SGA-1M protein. These methods include, for example, probing expression libraries with labeled SGA-1M protein, using SGA-1M protein in a manner similar to the well known technique of antibody probing of λgt11 libraries.

One method which detects protein interactions in vivo, the two-hybrid system, can be used. One version of this system has been described (Chien et al., 1991, supra) and is commercially available from Clontech (Palo Alto, Calif.).

### 5.5.3. Assays for Compounds that Interfere with SGA-1M Gene Product/Macromolecular Interaction

The SGA-1M gene product may, in vivo, interact with one or more macromolecules, such as proteins or nucleic acids. For purposes of this discussion, such macromolecules are referred to herein as “interacting partners”. In certain embodiments, the interacting partner is one that is identified according to the methods described in Section 5.5.2 above. In other embodiments, the interacting partner is a Nedd4 protein (see, e.g., Joliffe et al., 2000, J. Biochem. 351:557). Compounds that disrupt SGA-1M interactions in this way may be useful in regulating the activity of the SGA-1M gene product, including mutant SGA-1M gene products. Such compounds may include, but are not limited to small molecules and peptides, and the like, as described, for example, in Section 5.5.1. above, which would be capable of gaining access to the SGA-1M gene product. Thus the assays described below are useful in identifying proteins and or nucleic acids that can be used in methods to treat cancer. Proteins and nucleic acids that interact with SGA-1M can also be used in the diagnosis of cancer, e.g., breast cancer. Thus the assays described below are also useful in methods to diagnose cancer, e.g., breast cancer.

The basic principle of the assay systems used to identify compounds that interfere with the interaction between the SGA-1M gene product and its interacting partner or partners involves preparing a reaction mixture containing the SGA-1M gene product, and the interacting partner under conditions and for a time sufficient to allow the two to interact and bind, thus forming a complex. In order to test a compound for inhibitory activity, the reaction mixture is prepared in the presence and absence of the test compound. The test compound may be initially included in the reaction mixture, or may be added at a time subsequent to the addition of SGA-I M gene product and its intracellular interacting partner. Control reaction mixtures are incubated without the test compound or with a placebo. The formation of any complexes between the SGA-1M gene protein and the interacting partner is then detected. The formation of a complex in the control reaction, but not in the reaction mixture containing the test compound, indicates that the compound interferes with the interaction of the SGA-1M gene protein and the interacting partner. Additionally, complex formation within reaction mixtures containing the test compound and normal SGA-1M gene protein may also be compared to complex formation within reaction mixtures containing the test compound and a mutant SGA-1M gene protein. This comparison may be important in those cases wherein it is desirable to identify compounds that disrupt interactions of mutant but not normal SGA-1M gene proteins.

The assay for compounds that interfere with the interaction of the SGA-1M gene products and interacting partners can be conducted in a heterogeneous or homogeneous formnat. Heterogeneous assays involve anchoring either the SGA-1M gene product or the binding partner onto a solid phase and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between the SGA-1M gene products and the interacting partners, e.g., by competition, can be identified by conducting the reaction in the presence of the test substance; i.e., by adding the test substance to the reaction mixture prior to or simultaneously with the SGA-1M gene protein and intracellular interacting partner. Alternatively, test compounds that disrupt preformed complexes, e.g., compounds with higher binding constants that displace one of the components from the complex, can be tested by adding the test compound to the reaction mixture after complexes have been formed. The various formats are described briefly below.

In a heterogeneous assay system, either the SGA-1M gene product or the interacting partner, is anchored onto a solid surface, while the non-anchored species is labeled, either directly or indirectly. In practice, microtiter plates are conveniently utilized. The anchored species may be immobilized by non-covalent or covalent attachments. Non-covalent attachment may be accomplished simply by coating the solid surface with a solution of the SGA-1M gene product or interacting partner and drying. Alternatively, an immobilized antibody specific or selective for the species to be anchored may be used to anchor the species to the solid surface. The surfaces may be prepared in advance and stored.

In order to conduct the assay, the partner of the immobilized species is exposed to the coated surface with or without the test compound. After the reaction is complete, unreacted components are removed (e.g., by washing) and any complexes formed will remain immobilized on the solid surface. The detection of complexes anchored on the solid surface can be accomplished in a number of ways. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific or selective for the initially non-immobilized species (the antibody, in turn, may be directly labeled or indirectly labeled with a labeled anti-Ig antibody). Depending upon the order of addition of reaction components, test compounds which inhibit complex formation or which disrupt preformed complexes can be detected.

Alternatively, the reaction can be conducted in a liquid phase in the presence or absence of the test compound, the reaction products separated from unreacted components, and complexes detected; e.g., using an immobilized antibody specific or selective for one of the interacting components to anchor any complexes formed in solution, and a labeled antibody specific or selective for the other partner to detect anchored complexes. Again, depending upon the order of addition of reactants to the liquid phase, test compounds which inhibit complex or which disrupt preformed complexes can be identified.

In an alternate embodiment of the invention, a homogeneous assay can be used. In this approach, a preformed complex of the SGA-1M gene protein and the interacting partner is prepared in which either the SGA-1M gene product or its interacting partner is labeled, but the signal generated by the label is quenched due to complex formation (see, e.g., U.S. Pat. No. 4,109,496 by Rubenstein). The addition of a test substance that competes with and displaces one of the species from the preformed complex will result in the generation of a signal above background. In this way, test substances which disrupt SGA-1M gene protein/intracellular interacting partner interaction can be identified.

In a particular embodiment, the SGA-1M gene product can be prepared for immobilization using recombinant DNA techniques described in Section 5.1, above. For example, the SGA-1M coding region can be fused to a glutathione-S-transferase (GST) gene using a fusion vector, such as pGEX-5X-1, in such a manner that its interacting activity is maintained in the resulting fusion protein. The intracellular interacting partner can be purified and used to raise a monoclonal antibody, using methods routinely practiced in the art and described above, in Section 5.2. This antibody can be labeled with the radioactive isotope ¹²⁵I, for example, by methods routinely practiced in the art. In a heterogeneous assay, e.g., the GST-SGA-1M fusion protein can be anchored to glutathione-agarose beads. The intracellular interacting partner can then be added in the presence or absence of the test compound in a manner that allows interaction, e.g., binding, to occur. At the end of the reaction period, unbound material can be washed away, and the labeled monoclonal antibody can be added to the system and allowed to bind to the complexed components. The interaction between the SGA-1M gene protein and the intracellular interacting partner can be detected by measuring the amount of radioactivity that remains associated with the glutathione-agarose beads. A successful inhibition of the interaction by the test compound will result in a decrease in measured radioactivity.

Alternatively, the GST-SGA-1M gene fusion protein and the intracellular interacting partner can be mixed together in liquid in the absence of the solid glutathione-agarose beads. The test compound can be added either during or after the species are allowed to interact. This mixture can then be added to the glutathione-agarose beads and unbound material is washed away. Again the extent of inhibition of the SGA-1M gene product/interacting partner interaction can be detected by adding the labeled antibody and measuring the radioactivity associated with the beads.

In certain embodiments, assays for compounds that interfere with the interaction between an SGA-1M gene product and a binding partner are performed using full length SGA-1M(A) protein or a fusion protein comprising the SGA-1M(A) protein. In other embodiments, assays for compounds that interfere with the interaction between an SGA-1M gene product and a binding partner are performed using a portion of the SGA-1M(A) protein or a fusion protein comprising a portion of the SGA-1M(A) protein, for example a portion containing one, two or all three PY motifs of SGA-1M(A) (located at amino acids 39-42, 64-67, and 74-76 of the SGA-1M (A) open reading frame). Where the binding partner is a Nedd-4 protein, the entire Nedd-4 protein or a portion of the Nedd-4 protein containing one or more WW motifs can be used.

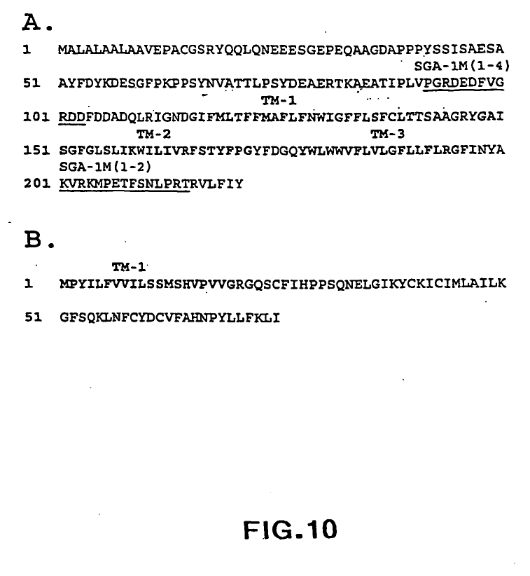

In other embodiments, assays for compounds that interfere with the interaction between an SGA-1M gene product and a binding partner are performed using full length SGA-1M(B) protein or a fusion protein comprising the SGA-1M(B) protein. In other embodiments, assays for compounds that interfere with the interaction between an SGA-1M gene product and a binding partner are performed using a portion of the SGA-1M(B) protein or a fusion protein comprising a portion of the SGA-1M(B) protein, for example a portion containing one or both CXXC motifs of SGA-1M(B) (see FIG. 10B).

### 5.5.4. Cell-Based Assays for Identification of Compounds which Modulate SGA-1M Activity

Cell-based methods are presented herein which identify compounds capable of treating e.g., breast cancer, ovarian cancer, skin cancer, a cancer of the lymphoid system, thyroid cancer, pancreatic cancer, stomach cancer, lung cancer and other cancers by modulating SGA-1M activity or expression levels. Specifically, such assays identify compounds which affect SGA-1M-dependent processes, such as but not limited to changes in cell morphology, cell division, differentiation, adhesion, motility, or phosphorylation, dephosphorylation of cellular proteins. Such assays can also identify compounds which affect SGA-1M expression levels or gene activity directly. Compounds identified via such methods can, for example, be utilized in methods for treating e.g., breast cancer, ovarian cancer, skin cancer, a cancer of the lymphoid system, thyroid cancer, pancreatic cancer, stomach cancer, lung cancer and other cancers, as well as metastases thereof.

In one embodiment, an assay is a cell-based assay in which a cell which expresses a membrane-bound form of the SGA-1M gene product, or a biologically active portion thereof, on the cell surface is contacted with a test compound and the ability of the test compound to bind to the polypeptide determined. In another embodiment the SGA-1M gene product is cytosolic. The cell, for example, can be a yeast cell or a cell of mammalian origin. Determining the ability of the test compound to bind to the polypeptide can be accomplished, for example, by coupling the test compound with a radioisotope or enzymatic label such that binding of the test compound to the polypeptide or biologically active portion thereofcan be determined by detecting the labeled compound in a complex. For example, test compounds can be labeled with ¹²⁵I, ³⁵S, ¹⁴C, or ³H, either directly or indirectly, and the radioisotope detected by direct counting of radio-emission or by scintillation counting. Alternatively, test compounds can be enzymatically labeled with, for example, horseradish peroxidase, alkaline phosphatase, or luciferase, and the enzymatic label detected by determination of conversion of an appropriate substrate to product. In a preferred embodiment, the assay comprises contacting a cell which expresses a membrane-bound form of a polypeptide of the invention, or a biologically active portion thereof, on the cell surface with a known compound which binds the polypeptide to form an assay mixture, contacting the assay mixture with a test compound, and determining the ability of the test compound to interact with the polypeptide, wherein determining the ability of the test compound to interact with the polypeptide comprises determining the ability of the test compound to preferentially bind to the polypeptide or a biologically active portion thereof as compared to the known compound.

In another embodiment, the cell-based assays are based on expression of the SGA-1M gene product in a mammalian cell and measuring the SGA-1M-dependent process. Any mammalian cells that can express the SGA-1M gene and allow the finctioning of the SGA-1M gene product can be used, in particular, cancer cells derived from the breast, such as MCF-7, BT483, Hs578T, HTB26, BT20 and T47D. Normal mammary gland cell lines such as, for example, CRL7030 and Hs578Bst, may also be used provided that an SGA-1M gene product is produced. Other mammalian cell lines that can be used include, but are not limited to CHO, HeLa, NIH3T3, and Vero. Recombinant expression of the SGA-1M gene in these cells can be achieved by methods described in Section 5.2. In these assays, cells producing functional SGA-1M gene products are exposed to a test compound for an interval sufficient for the compound to modulate the activity of the SGA-1M gene product. The activity of SGA-1M gene product can be measured directly or indirectly through the dectetion or measurement of SGA-1M-dependent cellular processes. As a control, a cell not producing the SGA-1M gene product may be used for comparisons. Depending on the cellular process, any techniques known in the art may be applied to detect or measure it.

In another embodiment a cell or cell line which is capable of expressing SGA-1M is contacted with a test compound which is believed to modulate expression of the SGA-1M gene. Expression levels of the SGA-1M gene can be monitored in the presence or absence of the test compound. Alternatively expression levels can be monitored in the presence of a test compound as compared to expression levels of the SGA-1M gene in the presence of a control compound or a placebo. Any method known in the art can be used to monitor SGA-1M gene expression. As an example, but not as a limitation, such methods can include Western blot, Northern Blot, and quantitative RT-PCR.

In yet another embodiment cells which express the SGA-1M gene product, e.g., MCF-7 cells are made permeable, e.g., by treatment with a mild detergent and exposed to a test compound. Binding of the test compound can be detected directly (e.g., radioactively labeling the test compound) or indirectly (antibody detection) or by any means known in the art.

In one embodiment, a cellular assay for SGA-1M activity entails examining the effect of a test compound on the subcellular localization of a SGA-1M gene product. In one embodiment, the effect of a test compound is assayed to determine whether the compound alters the subcellular localization of SGA-1M(A) protein from the endoplasmic reticulum or Golgi apparatus to a different cellular compartment (e.g., to the cytoplasm or the plasma membrane)

In another embodiment, a cellular assay for SGA-1M activity entails examining the effect of a test compound on the extent of ubiquitination of an SGA-1M(A) protein. A compound that results in altered ubiquitination of SGA-1M(A) protein may modulate the interaction between an SGA-1M(A) protein and its binding partners, for example a Nedd-4 protein. Such compounds can be assayed for their direct effect on SGA-1M(A), for example by testing their binding to SGA-1M(A) protein.

In another embodiment, a cellular assay for SGA-1M activity entails examining the effect of a test compound on the activity of a sodium channel, for example the amelioride-sensitive epithelial sodium channel (ENaC) (see, e.g., Harvey et al., 2001, J. Biol. Chem. 276:8597-8601). Once a compound is identified that has a modulatory effect on a sodium channel, the binding of the compound to SGA-1M(A) protein is assayed to determine whether it exerts its effect through SGA-1M(A). Alternatively, to determine whether a test compound exerts its effect on sodium channel activity through SGA-1M(A), the effect of the compound is compared in cellular systems that express SGA-1M(A) protein with cellular systems that do not express SGA-1M(A), and a compound is likely to exert its effect on sodium channel activity through SGA-1M(A) if it only modulated sodium channel activity in SGA-1M(A)-expressing cells. Examples of sodium channel assays include assays of membrane localization and patch clamp assays in Xenopus oocytes (Harvey et al., supra).

Any compound can be used in a cell based assay to test if it affects SGA-1M activity or expression levels. The compound can be a protein, a peptide, a nucleic acid, an antibody or fragment thereof, a small molecule, an organic molecule or an inorganic molecule. (e.g., steroid, pharmaceutical drug). A small molecule is considered a non-peptide compound with a molecular weight of less than 500 daltons.

### 5.6. Methods for Treatment of Cancer

Described below are methods and compositions for treating cancer e.g., breast cancer, ovarian cancer, skin cancer, a cancer of the lymphoid system, thyroid cancer, pancreatic cancer, stomach cancer, or lung cancer, using the SGA-1M gene or gene product as a therapeutic target. The outcome of a treatment is to at least produce in a treated subject a healthful benefit, which in the case of cancer, including breast cancer, includes but is not limited to remission of the cancer, palliation of the symptoms of the cancer, and/or control of metastatic spread of the cancer.

All such methods comprise methods which modulate SGA-1M gene activity and/or expression which in turn modulate the phenotype of the treated cell.

As discussed, above, successful treatment of cancers, e.g., breast cancer, ovarian cancer, skin cancer, a cancer of the lymphoid system, thyroid cancer, pancreatic cancer, stomach cancer, or lung cancer, can be brought about by techniques which serve to decrease SGA-1M activity. Activity can be decreased by, for example, directly decreasing SGA-1M gene product activity and/or by decreasing the level of SGA-1M gene expression. Thus the invention provides methods of treating a subject with cancer by administering to said subject an effective amount of a compound that antagonizes an SGA-1M gene product.

For example, compounds such as those identified through assays described, above, in Section 5.5, above, which decrease SGA-1M activity can be used in accordance with the invention to treat breast cancer or other cancers, e.g., ovarian cancer, skin cancer, a cancer of the lymphoid system, thyroid cancer, pancreatic cancer, stomach cancer, or Jung cancer. As discussed in Section 5.5, above, such molecules can include, but are not limited to proteins, nucleic acids, peptides, including soluble peptides, and small organic or inorganic molecules, and can be referred to as SGA-1M antagonists. Techniques for the determination of effective doses and administration of such compounds are described, below, in Section 5.7.

Further, antisense and ribozyme molecules which inhibit SGA-1M gene expression can also be used in accordance with the invention to reduce the level of SGA-1M gene expression, thus effectively reducing the level of SGA-1M gene product present, thereby decreasing the level of SGA-1M activity. The invention therefore relates to a pharmaceutical composition comprising an SGA-1M gene product. Still further, triple helix molecules can be utilized in reducing the level of SGA-1M gene activity. Such molecules can be designed to reduce or inhibit either wild type, or if appropriate, mutant target gene activity. Small organic or inorganic molecules can also be used to inhibit SGA-1M gene expression and/or inhibit production or activity of an SGA-1M gene product. Techniques for the production and use of such molecules are well known to those of skill in the art.

### 5.6.1. Antisense Molecules

Anti-sense nucleic acid molecules which are complementary to nucleic acid sequences contained within the SGA-1M gene as shown in FIG. 2 (SEQ ID NO:1), including but not limited to anti-sense nucleic acid molecules complementary to SEQ ID NO:2 and SEQ ID NO:4, can be used to treat any cancer, in which the expression level of the SGA-1M gene is elevated in cancerous cells or tissue as compared to normal cells or tissue or a predetermined non-cancerous standard. Thus in one embodiment of the invention a method of treating breast cancer is provided whereby a patient suffering from breast cancer, ovarian cancer, skin cancer, a cancer of the lymphoid system, thyroid cancer, pancreatic cancer, stomach cancer, or lung cancer is treated with an effective amount of an SGA-1M anti-sense nucleic acid molecule.

Antisense approaches involve the design of oligonucleotides (either DNA or RNA) that are complementary to SGA-1M gene mRNA. The antisense oligonucleotides will bind to the complementary SGA-1M gene mRNA transcripts and prevent translation. Absolute complementarity, although preferred, is not required. A sequence “complementary” to a portion of an RNA, as referred to herein, means a sequence having sufficient complementarity to be able to hybridize with the non-poly A portion of the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

Oligonucleotides that are complementary to the 5′ end of the message, e.g., the 5′ untranslated sequence up to and including the AUG initiation codon, should work most efficiently at inhibiting translation. However, sequences complementary to the 3′ untranslated sequences of mRNAs have also been shown to be effective at inhibiting translation of mRNAs as well. (See generally, Wagner, R., 1994, Nature 372:333). Thus, oligonucleotides complementary to the 5′-non-translated region, the 3′-non-translated region, or the non-translated, non-coding region between the two SGA-1M open reading frames of the SGA-1M gene (referred to herein after as the “intervening region”, as shown, for example, in FIG. 2, could be used in an antisense approach to inhibit translation of endogenous SGA-1M gene mRNA.

Oligonucleotides complementary to the 5′ untranslated region of the mRNA should include the complement of the AUG start codon. Antisense oligonucleotides complementary to mRNA coding regions are less efficient inhibitors of translation but could be used in accordance with the invention. Whether designed to hybridize to the 5′-, 3′-, intervening, or coding region of SGA-1M gene mRNA, antisense nucleic acids should be at least six nucleotides in length, and are preferably oligonucleotides ranging from 6 to about 50 nucleotides in length. In specific aspects the oligonucleotide is at least 10 nucleotides, at least 17 nucleotides, at least 25 nucleotides or at least 50 nucleotides.

Regardless of the choice of target sequence, it is preferred that in vitro studies are first performed to quantitate the ability of the antisense oligonucleotide to inhibit gene expression. It is preferred that these studies utilize controls that distinguish between antisense gene inhibition and nonspecific biological effects of oligonucleotides. It is also preferred that these studies compare levels of the target RNA or protein with that of an internal control RNA or protein. Additionally, it is envisioned that results obtained using the antisense oligonucleotide are compared with those obtained using a control oligonucleotide. It is preferred that the control oligonucleotide is of approximately the same length as the test oligonucleotide and that the nucleotide sequence of the oligonucleotide differs from the antisense sequence no more than is necessary to prevent specific hybridization to the target sequence.

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors in vivo), or agents facilitating transport across the cell membrane (see, e.g., Letsinger et al., 1989, Proc. Natl. Acad. Sci. USA 86:6553; Lemaitre et al., 1987, Proc. Natl. Acad. Sci. USA 84:648; PCT Publication No. WO88/09810, published Dec. 15, 1988) or the blood-brain barrier (see, e.g., PCT PublicationNo. WO89/10134, published Apr. 25, 1988), hybridization-triggered cleavage agents. (see, e.g., Krol el al., 1988, BioTechniques 6:958) or intercalating agents. (see, e.g., Zon, 1988, Pharm. Res. 5:539). To this end, the oligonucleotide may be conjugated to another molecule, e.g., a peptide, hybridization triggered cross-linking agent, transport agent, hybridization-triggered cleavage agent, etc.

The antisense oligonucleotide may comprise at least one modified base moiety which is selected from, the group including but not limited to 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine,5 ′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

The antisense oligonucleotide may also comprise at least one modified sugar moiety selected from the group including but not limited to arabinose, 2-fluoroarabinose, xylulose, and hexose.

In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet another embodiment, the antisense oligonucleotide is an α-anomeric oligonucleotide. An α-anomeric oligonucleotide forms specific double-stranded hybrids with complementary RNA in which, contrary to the usual β-units, the strands run parallel to each other (Gautier et al., 1987, Nucl. Acids Res. 15:6625). The oligonucleotide is a 2′-0-methylribonucleotide (Inoue et al., 1987, Nucl. Acids Res. 15:6131), or a chimeric RNA-DNA analogue (Inoue et al., 1987, FEBS Lett. 215:327).

The SGA-1M anti sense nucleic acid sequence can comprise the complement of any contiguous segment within the sequence of the SGA-1M gene (SEQ ID NO:1).

In one embodiment of the present invention, the SGA-1M antisense nucleic acid sequence is about 50 bp in length. In certain specific embodiments, the SGA-1M antisense nucleic acid sequence comprises the sequence complementary to nucleotides 1-50, 51-100, 101-150, 151-200, 201-250, 251-300, 301-350, 351-400, 401-450, 451-500, 501-550, 551-600, 601-650, 651-700, 701-750, 751-800, 801-850, 851-900, 901-950, 951-1000, 1001-1050, 1051-1100, 1101-1150, 1151-1200, 1201-1250, 1251-1300, 1301-1350, 1351-1400, 1401-1450, 1451-1500, 1501-1550, 1551-1600, 1601-1650, 1651-1700, 1701-1750, 1751-1800, 1801-1850, or 1851-1900 of SEQ ID NO:1.

In another embodiment the SGA-1M antisense nucleic acid sequence is about 100 bp in length. In certain specific embodiments, the SGA-1M antisense nucleic acid sequence comprises the sequence from nucleotides 1-100, 51-150, 101-200, 151-250, 201-300, 251-350, 301-400, 351-450, 401-500, 451-550, 501-600, 551-650, 601-700, 651-750, 701-800, 75-850, 801-900, 851-950, 901-1000, 951-1050, 1001-1100, 1051-1150, 1101-1200, 1151-1250, 1201-1300, 1251-1350, 1301-1400, 1351-1450, 1401-1500, 1451-1550, 1501-1600, 1551-1650, 1601-1700, 1651-1750, 1701-1800, 1751-1850, or 1801-1900 of SEQ ID NO:1.

In another embodiment the SGA-1M antisense nucleic acid sequence is about 200 bp in length. In a particular embodiment, the SGA-1M antisense nucleic acid sequence comprises the sequence from nucleotides 1-200, 101-300, 201-400, 301-500, 401-600, 501-700, 601-800, 701-900, 801-1000, 901-1100, 1001-1200, 1101-1300, 1201-1400, 1301-1500, 1401-1600, 1501-1700, 1601-1800 or 1701-1900 of SEQ ID NO: 1.

In another embodiment the SGA-1M antisense nucleic acid sequence is about 400 bp in length. In a particular embodiment, the SGA-1M antisense nucleic acid sequence comprises the sequence from nucleotides 1-400, 101-500, 201-600, 301-700, 401-800, 501-900, 601-1000, 701-1100, 801-1200, 901-1300, 1001-1400, 1101-1500, 1201-1600, 1301-1700, 1401-1800, or 1501-900 of SEQ ID NO:1.

Oligonucleotides of the invention may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein et al. (1988, Nucl. Acids Res. 16:3209), methylphosphonate oligonucleotides can be prepared by use of controlled pore glass polymer supports (Sarin et al., 1988, Proc. Natl. Acad. Sci. U.S.A. 85:7448), etc.

While antisense nucleotides complementary to the SGA-1M coding region could be used, those complementary to the transcribed untranslated region are most preferred.

The antisense molecules should be delivered to cells which express the SGA-1M gene in vivo. A number of methods have been developed for delivering anrtisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systemically.

However, it is often difficult to achieve intracellular concentrations of the antisense sufficient to suppress translation of endogenous mRNAs. Therefore a preferred approach utilizes a recombinant DNA construct in which the antisense oligonucleotide is placed under the control of a strong pot III or pol II promoter. The use of such a construct to transfect target cells in the patient will result in the transcription of sufficient amounts of single stranded RNAs that will form complementary base pairs with the endogenous SGA-1M gene transcripts and thereby prevent translation of the SGA-1M gene mRNA. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human cells. Such promoters can be inducible or constitutive. Such promoters include but are not limited to: the SV40 early promoter region (Bernoist and Chambon, 1981, Nature 290:304), the promoter contained in the 3′ long terminal repeat of Rous sarcoma virus (Yamamoto et al., 1980, Cell 22:787), the herpes thymidine kinase promoter (Wagner et al., 1981, Proc. Natl. Acad Sci. USA 78:1441), the regulatory sequences of the metallothionein gene (Brinster et al., 1982, Nature 296:39), etc. Any type of plasmid, cosmid, YAC or viral vector can be used to prepare the recombinant DNA construct which can be introduced directly into the tissue site. Alternatively, viral vectors can be used which selectively infect the desired tissue.

The effective dose of SGA-1M antisense oligonucleotide to be administered during a treatment cycle ranges from about 0.01 to 0.1, 0.1 to 1, or 1 to 10 mg/kg/day. The dose of SGA-1M antisense oligonucleotide to be administered can be dependent on the mode of administration. For example, intravenous administration of an SGA-1M antisense oligonucleotide would likely result in a significantly higher full body dose than a full body dose resulting from a local implant containing a pharmaceutical composition comprising SGA-1M antisense oligonucleotide. In one embodiment, an SGA-1M antisense oligonucleotide is administered subcutaneously at a dose of 0.01 to 10 mg/kg/day. In another embodiment, an SGA-1M antisense oligonucleotide is administered intravenously at a dose of 0.01 to 10 mg/kg/day. In yet another embodiment, an SGA-1M antisense oligonucleotide is administered locally at a dose of 0.01 to 10 mg/kg/day. It will be evident to one skilled in the art that local administrations can result in lower total body doses. For example, local administration methods such as intratumor administration, intraocular injection, or implantation, can produce locally high concentrations of SGA-1M antisense oligonucleotide, but represent a relatively low dose with respect to total body weight. Thus, in such cases, local administration of an SGA-1M antisense oligonucleotide is contemplated to result in a total body dose of about 0.01 to 5 mg/kg/day.

In another embodiment, a particularly high dose of SGA-1M antisense oligonucleotide, which ranges from about 10 to 50 mg/kg/day, is administered during a treatment cycle.

Moreover, the effective dose of a particular SGA-1M antisense oligonucleotide may depend on additional factors, including the type of disease, the disease state or stage of disease, the oligonucleotide's toxicity, the oligonucleotide's rate of uptake by cancer cells, as well as the weight, age, and health of the individual to whom the antisense oligonucleotide is to be administered. Because of the many factors present in vivo that may interfere with the action or biological activity of an SGA-1M antisense oligonucleotide, one of ordinary skill in the art can appreciate that an effective amount of an SGA-1M antisense oligonucleotide may vary for each individual.

In another embodiment, an SGA-1M antisense oligonucleotide is at a dose which results in circulating plasma concentrations of an SGA-1M antisense oligonucleotide which is at least 50 nM (nanomolar). As will be apparent to the skilled artisan, lower or higher plasma concentrations of an SGA-1M antisense oligonucleotide may be preferred depending on the mode of administration. For example, plasma concentrations of an SGA-1M antisense oligonucleotide of at least 50 nM can be appropriate in connection with intravenous, subcutaneous, intramuscular, controlled release, and oral administration methods, to name a few. In another example, relatively low circulating plasma levels ofan SGA-1M antisense oligonucleotide can be desirable, however, when using local administration methods such as, for example, intratumor administration, intraocular administration, or implantation, which nevertheless can produce locally high, clinically effective concentrations of SGA-1M antisense oligonucleotide.

The high dose may be achieved by several administrations per cycle. Alternatively, the high dose may be administered in a single bolus administration. A single administration of a high dose may result in circulating plasma levels of SGA-1M antisense oligonucleotide that are transiently much higher than 50 nM.

Additionally, the dose of an SGA-1M antisense oligonucleotide may vary according to the particular SGA-1M antisense oligonucleotide used. The dose employed is likely to reflect a balancing of considerations, among which are stability, localization, cellular uptake, and toxicity of the particular SGA-1M antisense oligonucleotide. For example, a particular chemically modified SGA-1M antisense oligonucleotide may exhibit greater resistance to degradation, or may exhibit higher affinity for the target nucleic acid, or may exhibit increased uptake by the cell or cell nucleus; all of which may permit the use of low doses. In yet another example, a particular chemically modified SGA-1M antisense oligonucleotide may exhibit lower toxicity than other antisense oligonucleotides, and therefore can be used at high doses. Thus, for a given SGA-1M antisense oligonucleotide, an appropriate dose to administer can be relatively high or relatively low. Appropriate doses would be appreciated by the skilled artisan, and the invention contemplates the continued assessment of optimal treatment schedules for particular species of SGA-1M antisense oligonucleotides. The daily dose can be administered in one or more treatments.

A “low dose” or “reduced dose” refers to a dose that is below the normally administered range, i.e., belowthe standard dose as suggested by the Physicians' Desk Reference, 54^{th} Edition (2000) or a similar reference. Such a. dose can be sufficient to inhibit cell proliferation, or demonstrates ameliorative effects in a human, or demonstrates efficacy with fewer side effects as compared to standard cancer treatments. Normal dose ranges used for particular therapeutic agents and standard cancer treatments employed for specific diseases can be found in the Physicians' Desk Reference. 54^{th} Edition (2000) or in Cancer: Principles & Practice of Oncology, DeVita, Jr., Hellman, and Rosenberg (eds.) 2nd edition, Philadelphia, Pa.: J. B. Lippincott Co., 1985.

Reduced doses of SGA-1M nucleic acid molecule, SGA-1M polypeptide, SGA-1M antagonist, and/or combination therapeutic can demonstrate reduced toxicity, such that fewer side effects and toxicities are observed in connection with administering an SGA-1M antagonist and one or more cancer therapeutics for shorter duration and/or at lower dosages when compared to other treatment protocols and dosage formulations, including the standard treatment protocols and dosage formulations as described in the Physicians' Desk Reference, 54^{th} Edition (2000) or in Cancer: Principles & Practice of Oncology, DeVita, Jr., Hellman, and Rosenberg (eds.) 2nd edition, Philadelphia, Pa.: J.B. Lippincott Co., 1985.

A “treatment cycle” or “cycle” refers to a period during which a single therapeutic or sequence of therapeutics is administered. In some instances, one treatment cycle may be desired, such as, for example, in the case where a significant therapeutic effect is obtained after one treatment cycle. The present invention contemplates at least one treatment cycle, generally preferably more than one treatment cycle.

Other factors to be considered in determining an effective dose of an SGA- I M antisense oligonucleotide include whether the oligonucleotide will be administered in combination with other therapeutics. In such cases, the relative toxicity of the other therapeutics may indicate the use of an SGA-1M antisense oligonucleotide at low doses. Alternatively, treatment with a high dose of SGA-1M antisense oligonucleotide can result in combination therapies with reduced doses of therapeutics. In a specific embodiment, treatment with a particularly high dose of SGA-1M antisense oligonucleotide can result in combination therapies with greatly reduced doses of cancer therapeutics. For example, treatment of a patient with 10, 20, 30, 40, or 50 mg/kg/day of an SGA-1M antisense oligonucleotide can further increase the sensitivity of a subject to cancer therapeutics. In such cases, the particularly high dose of SGA-1M antisense oligonucleotide is combined with, for example, a greatly shortened radiation therapy schedule. In another example, the particularly high dose of an SGA-1M antisense oligonucleotide produces significant enhancement of the potency of cancer therapeutic agents.

Additionally, the particularly high doses of SGA-1M antisense oligonucleotide may further shorten the period of administration of a therapeutically effective amount of SGA-1M antisense oligonucleotide and/or additional therapeutic, such that the length of a treatment cycle is much shorter than that of the standard treatment.

The invention contemplates other treatment regimens depending on the particular SGA-1M antisense oligonucleotide to be used, or depending on the particular mode of administration, or depending on whether an SGA-1M antisense oligonucleotide is administered as part of a combination therapy, e.g., in combination with a cancer therapeutic agent. The daily dose can be administered in one or more treatments.

### 5.6.2. Ribozyme Molecules

Ribozyme molecules which are complementary to RNA sequences coded for by the SGA-1M gene as shown in FIG. 2 can be used to treat cancer, including breast cancer, ovarian cancer, skin cancer, a cancer of the lymphoid system, thyroid cancer, pancreatic cancer, stomach cancer, or lung cancer.

Ribozymes are enzymatic RNA molecules capable of catalyzing the specific cleavage of RNA (For a review see, for example Rossi, J., 1994, Current Biology 4:469). The mechanism of ribozyme action involves sequence specific or selective hybridization of the ribozyme molecule to complementary target RNA, followed by a endonucleolytic cleavage. The composition of ribozyme molecules must include one or more sequences complementary to the target gene mRNA, and must include the well known catalytic sequence responsible for MRNA cleavage (See U.S. Pat. No. 5,093,246). As such, within the scope of the invention are engineered hammerhead motif ribozyme molecules that specifically and efficiently catalyze endonucleolytic cleavage of RNA sequences encoding target gene proteins. Ribozyme molecules designed to catalytically cleave SGA-1M mRNA transcripts can also be used to prevent translation of SGA-1M mRNA and expression of target or pathway gene. (See, e.g, PCT International Publication WO90/11364, published Oct. 4, 1990; Sarver et al., 1990, Science 247:1222). While ribozymes that cleave mRNA at site specific recognition sequences can be used to destroy SGA-1M mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Haseloff and Gerlach, 1988, Nature 334:585. Preferably the ribozyme is engineered so that the cleavage recognition site is located near the 5′ end of the SGA-1M mRNA; i.e., to increase efficiency and minimize the intracellular accumulation of non-functional mRNA transcripts.

The ribozymes of the present invention also include RNA endoribonucleases (hereinafter “Cech-type ribozymes”) such as the one which occurs naturally in Tetrahymena Thermophila (known as the IVS, or L-19 IVS RNA) and which has been extensively described by Thomas Cech and collaborators (Zaug et al., 1984, Science 224:574; Zaug and Cech, 1986, Science 231:470; Zaug et al., 1986, Nature 324:429; published International patent application No. WO 88/04300 by University Patents Inc.; Been and Cech, 1986, Cell47:207). The Cech-type ribozymes have an eight base pair active site which hybridizes to a target RNA sequence whereafter cleavage of the target RNA takes place. The invention encompasses those Cech-type ribozymes which target eight base-pair active site sequences that are present in an SGA-1M gene.

As in the antisense approach, the ribozymes can be composed of modified oligonucleotides (e.g., for improved stability, targeting, etc.) and should be delivered to cells which express the SGA-1M gene in vivo. A preferred method of delivery involves using a DNA construct “encoding” the ribozyme under the control of a strong constitutive pol III or pol II promoter, so that transfected cells will produce sufficient quantities of the ribozyme to destroy endogenous SGA-1M gene messages and inhibit translation. Because ribozymes unlike antisense molecules, are catalytic, a lower intracellular concentration is required for efficiency.

Anti-sense RNA and DNA, ribozyme, and triple helix molecules of the invention can be prepared by any method known in the art for the synthesis of DNA and RNA molecules. These include techniques for chemically synthesizing oligodeoxyribonucleotides and oligoribonucleotides well known in the art such as for example solid phase phosphoramidite chemical synthesis. Alternatively, RNA molecules can be generated by in vitro and in vivo transcription of DNA sequences encoding the antisense RNA molecule. Such DNA sequences can be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Various well-known modifications to the DNA molecules can be introduced as a means of increasing intracellular stability and half-life. Possible modifications include, but are not limited to, the addition of flanking sequences of ribo- or deoxy- nucleotides to the 5′ and/or 3′ ends of the molecule or the use of phosphorothioate or 2′ O-methyl rather than phospho-diesterase linkages within the oligodeoxyribonucleotide backbone.

### 5.6.3. Therapeutic Antibodies

Antibodies exhibiting capability to downregulate SGA-1M gene product activity can be utilized to treat breast cancer and other cancers, e.g., ovarian cancer, skin cancer, a cancer of the Iymphoid system, thyroid cancer, pancreatic cancer, stomach cancer, or lung cancer, in which the SGA-1M expression levels are elevated. Such antibodies can be generated using standard techniques described in Section 5.3, above, against full length wild type or mutant SGA-1M proteins, or against peptides corresponding to portions of the proteins. The antibodies include but are not limited to polyclonal, monoclonal, Fab fragments, single chain antibodies, chimeric antibodies, and the like.

Antibodies that recognize any epitope on the SGA-1M protein can be used as therapy against cancer. Because SGA-1M(A) and SGA-1M(B) contain multiple hydrophobic domains and a signal sequence, they may be expressed as a membrane bound protein. Thus, antibodies that recognize surface expressed epitopes of the SGA-1M(A) or SGA-1M(B) are useful to treat or prevent cancer.

Because SGA-1M(A) and SGA-1M(B) can also be expressed as an intracellular proteins, it is preferred that internalizing antibodies be used. However, lipofectin or liposomes can be used to deliver the antibody or a fragment of the Fab region which binds to the SGA-1M epitope into cells. Where fragments of the antibody are used, the smallest inhibitory fragment which binds to the SGA-1M(A) or SGA-1M(B) is preferred. For example, peptides having an amino acid sequence corresponding to the domain of the variable region of the antibody that binds to SGA-1M(A) or SGA-1M(B) can be used. Such peptides can be synthesized chemically or produced via recombinant DNA technology using methods well known in the art (e.g., see Creighton, 1983, supra; and Sambrook et al., 1989, supra). Alternatively, single chain antibodies, such as neutralizing antibodies, which bind to intracellular epitopes can also be administered. Such single chain antibodies can be administered, for example, by expressing nucleotide sequences encoding single-chain antibodies within the target cell population by utilizing, for example, techniques such as those described in Marasco et al. (Marasco,. et al., 1993, Proc. Natl. Acad Sci USA 90:7889).

The invention also contemplates the use of antibodies that are conjugated to a cytostatic and/or a cytotoxic agent in the methods of the invention, e.g., the treatment of cancer. A useful class of cytotoxic or cytostatic agents for practicing the therapeutic regimens of the present invention, by conjugation to an antibody, include, but are not limited to, the following non-mutually exclusive classes of agents: alkylating agents, anthracyclines, antibiotics, antifolates, antimetabolites, antitubulin agents, auristatins, chemotherapy sensitizers, DNA minor groove binders, DNA replication inhibitors, duocarmycins, etoposides, fluorinated pyrimidines, lexitropsins, nitrosoureas, platinols, purine antimetabolites, puromycins, radiation sensitizers, steroids, taxanes, topoisomerase inhibitors, and vinca alkaloids.

Individual cytotoxic or cytostatic agents encompassed by the invention include but are not limited to an androgen, anthramycin (AMC), asparaginase, 5-azacytidine, azathioprine, bleomycin, busulfan, buthionine sulfoximine, camptothecin, carboplatin, carmustine (BSNU), CC-1065, chlorambucil, cisplatin, colchicine, cyclophosphamide, cytarabine, cytidine arabinoside, cytochalasin B, dacarbazine, dactinomycin (formerly actinomycin), daunorubicin, decarbazine, docetaxel, doxorubicin, an estrogen, 5-fluordeoxyuridine, 5-fluorouracil, gramicidin D, hydroxyurea, idarubicin, ifosfamide, irinotecan, lomustine (CCNU), mechlorethamine, melphalan, 6-mercaptopurine, methotrexate, mithramycin, mitomycin C, mitoxantrone, nitroimidazole, paclitaxel, plicamycin, procarbizine, streptozotocin, tenoposide, 6-thioguanine, thioTEPA, topotecan, vinblastine, vincristine, vinorelbine, VP-16 and VM-26.

In a preferred embodiment, the cytotoxic or cytostatic agent is an antimetabolite. The antimetabolite can be a purine antagonist (e.g., azothioprine) or mycophenolate mofetil), a dihydrofolate reductase inhibitor (e.g., methotrexate), acyclovir, gangcyclovir, zidovudine, vidarabine, ribavarin, azidothymidine, cytidine arabinoside, amantadine, dideoxyuridine, iododeoxyuridine, poscarnet, and trifluridine.

In certain preferred embodiments, the cytotoxic agent conjugated to an anti-SGA-1M antibody is selected from the group consisting of an enediyne, a lexitropsin, a duocarnmycin, a taxane, a puromycin, a dolastatin, an auristatin, a maytansinoid, and a vinca alkaloid. In certain, more specific embodiments, the cytotoxic agent is paclitaxel, docetaxel, CC-1065, SN-38, topotecan, morpholino-doxorubicin, rhizoxin, cyanomorpholino-doxorubicin, dolastatin-10, echinomycin, combretastatin, calicheamicin, maytansine, DM-1, auristatin E, AEB, AEVB, AEFP, MMAE, or netropsin. The structures of AEB, AEVB, AEFP and MMAE and methods of making conjugating these cytotoxic agents to an antibody are described in U.S. provisional application Nos. 60/400,403, filed Jul. 31, 2002, and 60/427,897, filed Nov. 20, 2002, each of which is incorporated herein in its entirety.

In other preferred embodiments, the cytotoxic agent of an anti-SGA-1M antibody-cytotoxic agent conjugate is an anti-tubulin agent. In more specific embodiments, the cytotoxic agent is selected from the group consisting of a vinca alkaloid, a podophyllotoxin, a taxane, a baccatin derivative, a cryptophysin, a maytansinoid, a combretastatin, a dolastatin and an auristatin. In more specific embodiments, the cytotoxic agent is vincristine, vinblastine, vindesine, vinorelbine, VP-16, camptothecin, paclitaxel, docetaxel, epithilone A, epithilone B, nocodazole, colchicine, colcimid, estramustine, cemadotin, discodermolide, maytansine, DM-1, AEFP, auristatin E, AEB, AEVB, AEFP, MMAE or eleutherobin.

In certain specific embodiments, the anti-SGA-1M antibody of an anti-SGA-1M antibody-cytotoxic agent conjugate of the invention is conjugated to the cytotoxic agent via a linker, wherein the linker is peptide linker. In specific embodiments, the anti-SGA-1M antibody of an anti-SGA-1M antibody-cytotoxic agent conjugate of the invention is conjugated to the cytotoxic agent via a linker, wherein the linker is a valine-citrulline (val-cit) linker, a phenylalanine-lysine (phe-lys) linker, a hydrazone linker, or a disulfide linker. In certain embodiments, the anti-SGA-1M antibody ofan anti-SGA-1M antibody-cytotoxic agent conjugate of the invention is conjugated to the cytotoxic agent via a peptide linker.

In certain embodiments, the anti-SGA-1M antibody of an anti-SGA-1M antibody-cytotoxic agent conjugate of the invention is conjugated to the cytotoxic agent via a linker, wherein the linker is hydrolyzable at a pH of less than 5.5. In a specific embodiment the linker is hydrolyzable at a pH of less than 5.0.

In certain embodiments, the anti-SGA-1M antibody of an anti-SGA-1M antibody-cytotoxic agent conjugate of the invention is conjugated to the cytotoxic agent via a linker, wherein the linker is cleavable by a protease. In a specific embodiment, the protease is a lysosomal protease. In other specific embodiments, the protease is, inter alia, a membrane-associated protease, an intracellular protease, or an endosomal protease.

Techniques for conjugating such therapeutic moieties to proteins, and in particular to antibodies, are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc., 1985); Hellstrom et al., “Antibodies For Drug Delivery”, in Controlled Drug Delivery (2nd ed.), Robinson etal. (eds.), pp.623-53 (Marcel Dekker, Inc., 1987); Thorpe, “Antibody Carriers Of Cytotoxic Agents In Cancer Therapy: A Review”, in Monoclonal Antibodies '84: Biological And Clinical Applications, Pinchera el al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., 1982, Immunol. Rev. 62:119-58.

### 5.6.4. Targeted Disruption of SGA-1M Expression

As briefly described in Section 5.2.4, supra, endogenous SGA-1M gene expression can also be reduced by inactivating or “knocking out” the gene or its promoter using targeted homologous recombination. (e.g., see Smithies et al., 1985, Nature 317:230; Thomas & Capecchi, 1987, Cell 51:503; Thompson et al., 1989 Cell 5:313). For example, a mutant, non-functional SGA-1M gene (or a completely unrelated DNA sequence) flanked by DNA homologous to the endogenous SGA-1M gene (either the coding regions or regulatory regions of the SGA-1M gene) can be used, with or without a selectable marker and/or a negative selectable marker, to transfect cells that express SGA-1M gene in vivo. Insertion of the DNA construct, via targeted homologous recombination, results in inactivation of the SGA-1M gene. Such approaches are particularly suited where modifications to ES (embryonic stem) cells can be used to generate animal offspring with an inactive SGA-1M gene homolog (e.g., see Thomas & Capecchi 1987 supra and Thompson 1989, supra). Such techniques can also be utilized to generate animal models of breast cancer and other types of cancer. It should be noted that this approach can be adapted for use in humans provided the recombinant DNA constructs are directly administered or targeted to the required site in vivo using appropriate vectors, e.g., herpes virus vectors, retrovirus vectors, adenovirus vectors, or adeno associated virus vectors.

Alternatively, endogenous SGA-1M gene expression can be reduced by targeting deoxyribonucleotide sequences complementary to the regulatory region of the SGA-1M gene (i. e., the SGA-1M gene promoter and/or enhancers) to form triple helical structures that prevent transcription of the SGA-1M gene in target cells in the body. (See generally, Helene, 1991, Anticancer Drug Des. 6(6):569; Helene et al., 1992, Ann, N.Y. Acad Sci. 660:27; and Maher, 1992, Bioassays 14(12):807).

### 5.6.5. Combinaiton Therapies

The administration of an SGA-1M antagonist can potentiate the effect of anti-cancer agents. In a preferred embodiment, the invention further encompasses the use of combination therapy to prevent or treat cancer. In one embodiment, the SGA-1M antagonist selectively or specifically antagonizes SGA-1M(A) relative to SGA-1M(B) expression oractivity. In another embodiment, the SGA-1M antagonist selectively or specifically antagonizes SGA-1M(B) relative to SGA-1M(A) expression or activity. In yet another embodiment, the SGA-1M antagonist antagonizes both SGA-1M(A) and SGA-1M(B) expression or activity.

In one embodiment, breast cancer and other cancers (e.g., breast cancer, ovarian cancer, skin cancer, a cancer of the lymphoid system, thyroid cancer, pancreatic cancer, stomach cancer, or lung cancer) can be treated with a pharmaceutical composition comprising an SGA-1M antagonist in combination with 5-fluorouracil, cisplatin, docetaxel, doxorubicin, Herceptin®, gemcitabine (Seidman, 2001, Oncology 15:11-14), IL-2, paclitaxel, and/or VP-16 (etoposide).

These combination therapies can also be used to prevent cancer, prevent the recurrence of cancer, or prevent the spread or metastasis or cancer.

Combination therapy also includes, in addition to administration of an SGA-1M antagonist, the use of one or more molecules, compounds or treatments that aid in the prevention or treatment of cancer (i.e., cancer therapeutics), which molecules, compounds or treatments includes, but is not limited to, chemoagents, immunotherapeutics, cancer vaccines, anti-angiogenic agents, cytokines, hormone therapies, gene therapies, and radiotherapies.

In one embodiment, one or more chemoagents, in addition to an SGA-1M antagonist, is administered to treat a cancer patient. A chemoagent (or “anti-cancer agent” or “anti-tumor agent” or “cancer therapeutic”) refers to any molecule or compound that assists in the treatment of tumors or cancer. Examples of chemoagents contemplated by the present invention include, but are not limited to, cytosine arabinoside, taxoids (e.g., paclitaxel, docetaxel), anti-tubulin agents (e.g., paclitaxel, docetaxel, epothilone B, or its analogues), macrolides (e.g., rhizoxin ) cisplatin, carboplatin, adriamycin, tenoposide, mitozantron, discodermolide, eleutherobine, 2-chlorodeoxyadenosine, alkylating agents (e.g., cyclophosphamide, mechlorethamine, thioepa, chlorambucil, melphalan, carmustine (BSNU), lomustine (CCNU), cyclothosphamide, busulfan, dibromomannitol, streptozotocin, mitomycin C, and cis-dichlorodiamine platinum (II) (DDP) cisplatin, thio-tepa), antibiotics (e.g., dactinomycin (formerly actinomycin), bleomycin, mithramycin, anthramycin), antimetabolites (e.g., methotrexate, 6-mercaptopurine, 6-thioguanine, cytarabine, flavopiridol, 5-fluorouracil, fludarabine, gemcitabine, dacarbazine, temozolamide), asparaginase, Bacillus Calmette and Guerin, diphtheria toxin, hexamethylmelamine, hydroxyurea, LYSODREN®, nucleoside analogues, plant alkaloids (e.g., Taxol, paclitaxel, camptothecin, topotecan, irinotecan (CAMPTOSAR, CPT-11), vincristine, vinca alkyloids such as vinblastine), podophyllotoxin (including derivatives such as epipodophyllotoxin, VP-16 (etoposide), VM-26 (teniposide)), cytochalasin B, coichine, gramicidin D, ethidium bromide, emetine, mitomycin, procarbazine, mechlorethamine, anthracyclines (e.g., daunorubicin (formerly daunomycin), doxorubicin, doxorubicin liposomal), dihydroxyanthracindione, mitoxantrone, mithramycin, actinomycin D, procaine, tetracaine, lidocaine, propranolol, puromycin, anti-mitotic agents, abrin, ricin A, pseudomonas exotoxin, nerve growth factor, platelet derived growth factor, tissue plasminogen activator, aldesleukin, allutamine, anastrozle, bicalutamide, biaomycin, busulfan, capecitabine, carboplain, chlorabusil, cladribine, cylarabine, daclinomycin, estramnusine, floxuridhe, gamcitabine, gosereine, idarubicin, itosfamide, lauprolide acetate, levamisole, lomusline, mechlorethanine, magestrol, acetate, mercaptopurino, mesna, rnitolanc, pegaspergase, pentoslatin, picamycin, riuxlmab, campath-1, straplozocin, thioguanine, tretinoin, vinorelbine, or any fragments, family members, or derivatives thereof, including pharmaceutically acceptable salts thereof. Compositions comprising one or more chemoagents (e.g., FLAG, CHOP) are also contemplated by the present invention. FLAG comprises fludarabine, cytosine arabinoside (Ara-C) and G-CSF. CHOP comprises cyclophosphamide, vincristine, doxorubicin, and prednisone.

In one embodiment, said chemoagent is gemcitabine at a dose ranging from 100 to 1000 mg/m²/cycle. In one embodiment, said chemoagent is dacarbazine at a dose ranging from 200 to 4000 mg/m²/cycle. In a preferred embodiment, said dose ranges from 700 to 1000 mg/m²/cycle. In another embodiment, said chemoagent is fludarabine at a dose ranging from 25 to 50 mg/m²/cycle. In another embodiment, said chemoagent is cytosine arabinoside (Ara-C) at a dose ranging from 200 to 2000 mg/m²/cycle. In another embodiment, said chemoagent is docetaxel at a dose ranging from 1.5 to 7.5 mg/kg/cycle. In another embodiment, said chemoagent is paclitaxel at a dose ranging from 5 to 15 mg/kg/cycle. In yet another embodiment, said chemoagent is cisplatin at a dose ranging from 5 to 20 mg/kg/cycle. In yet another embodiment, said chemoagent is 5-fluorouracil at a dose ranging from 5 to 20 mg/kg/cycle. In yet another embodiment, said chemoagent is doxorubicin at a dose ranging from 2 to 8 mg/kg/cycle. In yet another embodiment, said chemoagent is epipodophyllotoxin at a dose ranging from 40 to 160 mg/kg/cycle. In yet another embodiment, said chemoagent is cyclophosphamide at a dose ranging from 50 to 200 mg/kg/cycle. In yet another embodiment, said chemoagent is irinotecan at a dose ranging from 50 to 75, 75 to 100, 100 to 125, or 125 to 150 mg/m²/cycle. In yet another embodiment, said chemoagent is vinblastine at a dose ranging from 3.7 to 5.4, 5.5 to 7.4, 7.5 to 11, or 11 to 18.5 mg/m²/cycle. In yet another embodiment, said chemoagent is vincristine at a dose ranging from 0.7 to 1.4, or 1.5 to 2 mg/m²/cycle. In yet another embodiment, said chemoagent is methotrexate at a dose ranging from 3.3 to 5, 5 to 10, 10 to 100, or 100 to 1000 mg/m²/cycle.

In a preferred embodiment, the invention further encompasses the use of low doses of chemoagents when administered as part of an SGA-1M antagonist treatment regimen. For example, initial treatment with an SGA-1M antagonist increases the sensitivity of a tumor to subsequent challenge with a dose of chemoagent, which dose is near or below the lower range of dosages when the chemoagent is administered without an SGA-1M antagonist. In one embodiment, an SGA-1M antagonist and a low dose (e.g., 6 to 60 mg/m²/day or less) of docetaxel are administered to a cancer patient. In another embodiment, an SGA-1M antagonist and a low dose (e.g., 10 to 135 mg/m²/day or less) of paclitaxel are administered to a cancer patient. In yet another embodiment, an SGA-1M antagonist and a low dose (e.g., 2.5 to 25 mg/m²/day or less) of fludarabine are administered to a cancer patient. In yet another embodiment, an SGA-1M antagonist and a low dose (e.g., 0.5 to 1.5 g/m²/day or less) of cytosine arabinoside (Ara-C) are administered to a cancer patient.

The invention, therefore, contemplates the use of one or more SGA-1M antagonists, which is administered prior to, subsequently, or concurrently with low doses of chemoagents, for the prevention or treatment of cancer.

In one embodiment, said chemoagent is gemcitabine at a dose ranging from 10 to 100 mg/m²/cycle.

In one embodiment, said chemoagent is cisplatin, e.g., PLATFNOL™ or PLATINOL-AQ™(Bristol Myers), at a dose ranging from 5 to 10, 10 to 20, 20 to 40, or 40 to 75 mg/m²/cycle. In another embodiment, a dose of cisplatin ranging from 7.5 to 75 mg/m²/cycle is administered to a patient with ovarian cancer or other cancer. In another embodiment, a dose of cisplatin ranging from 5 to 50 mg/m²/cycle is administered to a patient with bladder cancer or other cnacer.

In another embodiment, said chemoagent is carboplatin, e.g., PARAPLATIN™(Bristol Myers), at a dose ranging from 2 to 4, 4 to 8, 8 to 16, 16 to 35, or 35 to 75 mg/m²/cycle. In another embodiment, a dose of carboplatin ranging from 7.5 to 75 mg/m²/cycle is administered to a patient with ovarian cancer or other cancer. In another embodiment, a dose of carboplatin ranging from 5 to 50 mg/m²/cycle is administered to a patient with bladder cancer or other cancer. In another embodiment, a dose of carboplatin ranging from 2 to 20 mg/m²/cycle is administered to a patient with testicular cancer or other cnacer.

In another embodiment, said chemoagent is docetaxel, e.g., TAXOTERE™ (Rhone Poulenc Rorer) at a dose ranging from 6 to 10, 10 to 30, or 30 to 60 mg/m²/cycle.

In another embodiment, said chemoagent is paclitaxel, e.g., TAXOLT™ (Bristol Myers Squibb), at a dose ranging from 10 to 20, 20 to 40, 40 to 70, or 70 to 135 mg/kg/cycle.

In another embodiment, said chemoagent is 5-fluorouracil at a dose ranging from 0.5 to 5 mg/kg/cycle.

In another embodiment, said chemoagent is doxorubicin, e.g., ADRIAMYCIN™ (Pharmacia & Upjohn), DOXIL (Alza), RUBEX™ (Bristol Myers Squibb), at a dose ranging from 2 to 4,4 to 8,8 to 15, 15 to 30, or 30 to 60 mg/kg/cycle.

In another embodiment, an SGA-1M antagonist is administered in combination with one or more immunotherapeutic agents, such as antibodies and immunomodulators, which includes, but is not limited to, Herceptin®, Retuxan®, OvaRex, Panorex, BEC2, IMC-C225, Vitaxin, Campath I/H, Smart MI95, LymphoCide, SmartID10, and Oncolym, rituxan, rituximab, gemtuzumab, or trastuzumab.

In another embodiment, an SGA-1M antagonist is administered in combination with one or more anti-angiogenic agents, which includes, but is not limited to, angiostatin, thalidomide, kringle 5, endostatin, Serpin (Serine Protease Inhibitor) anti-thrombin, 29 kDa N-terminal and a 40 kDa C-terminal proteolytic fragments of fibronectin, 16 kDa proteolytic fragment of prolactin, 7.8 kDa proteolytic fragment of platelet factor-4, a 13-amino acid peptide corresponding to a fragment ofplatelet factor-4 (Maione et al., 1990, Cancer Res. 51:2077), a 14-amino acid peptide corresponding to a fragment of collagen I (Tolma et al., 1993, J. Cell Biol. 122:497), a 19 amino acid peptide corresponding to a fragment of Thrombospondin I (Tolsma et al., 1993, J. Cell. Biol. 122:497), a 20-amino acid peptide corresponding to a fragment of SPARC (Sage et al., 1995, J Cell. Biochem. 57:1329-), or any fragments, family members, or derivatives thereof, including pharmaceutically acceptable salts thereof.

Other peptides that inhibit angiogenesis and correspond to fragments of laminin, fibronectin, procollagen, and EGF have also been described (See the review by Cao, 1998, Prog. Mol. Subcell. Biol. 20:161). Monoclonal antibodies and cyclic pentapeptides, which block certain integrins that bind RGD proteins (i.e., possess the peptide motif Arg-Gly-Asp), have been demonstrated to have anti-vascularization activities (Brooks et al., 1994, Science 264:569; Hamnmes et al., 1996, Nature Medicine 2:529). Moreover, inhibition of the urokinase plasminogen activator receptor by receptor antagonists inhibits angiogenesis, tumor growth and metastasis (Min et al., 1996, Cancer Res. 56:2428-33; Crowley et al., 1993, Proc Natl Acad Sci. USA 90:5021). Use of such anti-angiogenic agents is also contemplated by the present invention.

In another embodiment, an SGA-1M antagonist is administered in combination with a regimen of radiation.

In another embodiment, an SGA-1M antagonist is administered in combination with one or more cytokines, which includes, but is not limited to, lymphokines, tumor necrosis factors, tumor necrosis factor-like cytokines, lymphotoxin-a, lymphotoxin-b, interferon-a, interferon-b, macrophage inflammatory proteins, granulocyte monocyte colony stimulating factor, interleukins (including, but not limited to, interleukin-1, interleukin-2, interleukin-6, interleukin-12, interleukin-15, interleukin-18), OX40, CD27, CD30, CD40 or CD137 ligands, Fas-Fas ligand, 4-1BBL, endothelial monocyte activating protein or any fragments, family members, or derivatives thereof, including pharmaceutically acceptable salts thereof.

In yet another embodiment, an SGA-1M antagonist is administered in combination with a cancer vaccine. Examples of cancer vaccines include, but are not limited to, autologous cells or tissues, non-autologous cells or tissues, carcinoembryonic antigen, alpha-fetoprotein, human chorionic gonadotropin, BCG live vaccine, melanocyte lineage proteins (e.g., gp100, MART-1/MelanA, TRP-1 (gp75), tyrosinase, widely shared tumor-associated, including tumor-specific, antigens (e.g., BAGE, GAGE-1, GAGE-2, MAGE-1, MAGE-3, N-acetylglucosaminyltransferase-V, p15), mutated antigens that are tumor-associated (β-catenin, MUM-1, CDK4), nonmelanoma antigens (e.g., HER-2/neu (breast and ovarian carcinoma), human papillomavirus-E6, E7 (cervical carcinoma), MUC-1 (breast, ovarian and pancreatic carcinoma). For human tumor antigens recognized by T cells, see generally Robbins and Kawakami, 1996, Curr. Opin. Immunol. 8:628. Cancer vaccines may or may not be purified preparations.

In yet another embodiment, an SGA-1M antagonist is used in association with a hormonal treatment. Hormonal therapeutic treatments comprise hormonal agonists, hormonal antagonists (e.g., flutamide, tamoxifen, leuprolide acetate (LUPRON), LH-RH antagonists), inhibitors of hormone biosynthesis and processing, and steroids (e.g., dexamethasone, retinoids, betamethasone, cortisol, cortisone, prednisone, dehydrotestosterone, glucocorticoids, mineralocorticoids, estrogen, testosterone, progestins), antigestagens (e.g., mifepristone, onapristone), and antiandrogens (e.g., cyproterone acetate).

In yet another embodiment, an SGA-1M antagonist is used in association with a gene therapy program in the treatment of cancer. In one embodiment, gene therapy with recombinant cells secreting interleukin-2 is administered in combination with an SGA-1M antagonist to prevent or treat cancer, particularly breast cancer (See, e.g., Deshmukh et al., 2001, J. Neurosurg. 94:287).

In one embodiment, an SGA-1M antagonist is administered, in combination with at least one cancer therapeutic agent, for a short treatment cycle to a cancer patient to treat cancer. The duration of treatment with the cancer therapeutic agent may vary according to the particular cancer therapeutic agent used. The invention also contemplates discontinuous administration or daily doses divided into several partial administrations. An appropriate treatment time for a particular cancer therapeutic agent will be appreciated by the skilled artisan, and the invention contemplates the continued assessment of optimal treatment schedules for each cancer therapeutic agent.

The present invention contemplates at least one cycle, preferably more than one cycle during which a single therapeutic or sequence oftherapeutics is administered. An appropriate period of time for one cycle will be appreciated by the skilled artisan, as will the total number of cycles, and the interval between cycles. The invention contemplates the continued assessment of optimal treatment schedules for each SGA-1M antagonist and cancer therapeutic agent.

### 5.7. Pharmaceutical Preparations and Methods of Administration

The compounds, proteins, peptides, nucleic acid sequences and fragments thereof, described herein can be administered to a patient at therapeutically effective doses to treat cancer, e.g., breast cancer, ovarian cancer, skin cancer, a cancer of the lymphoid system, thyroid cancer, pancreatic cancer, stomach cancer, or lung cancer, in which the expression level of the SGA-1M gene is elevated compared to a non-cancerous sample or a predetermined non-cancerous standard. A therapeutically effective dose refers to that amount of a compound sufficient to result in a healthful benefit in the treated subject.

### 5.7.1. Effective Dose

Toxicity and therapeutic efficacy of compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred. While compounds that exhibit toxic side effects can be used, care should be taken to design a delivery system that targets such compounds to the site of affected tissue in order to minimize potential damage to unaffected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma can be measured by any technique known in the art, for example, by high performance liquid chromatography.

### 5.7.2. Formulations and Use

The invention relates to pharmaceutical compositions, including, but not limited to pharmaceutical compositions comprising an SGA-1M gene product, or antagonists thereof, for the treatment or prevention of cancer.

Pharmaceutical compositions for use in accordance with the present invention, e. g., methods to treat or prevent cancer, can be formulated in a conventional manner using one or more physiologically acceptable carriers or excipients.

Thus, the compounds and their physiologically acceptable salts and solvents can be formulated for administration by inhalation or insufflation (either through the mouth or the nose) or oral, buccal, parenteral or rectal administration.

For oral administration, the pharmaceutical compositions can take the form of, for exarnple, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g. sodium lauryl sulphate). The tablets can be coated by methods well known in the art. Liquid preparations for oral administration can take the form of, for example, solutions, syrups or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g, methyl or propyl-p-hydroxybenzoates or sorbic acid). The preparations can also contain buffer salts, flavoring, coloring and sweetening agents as appropriate.

Preparations for oral administration can be suitably formulated to give controlled release of the active compound.

For buccal administration the compositions can take the form oftablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

The compounds can be formulated for parenteral administration (ie., intravenous or intramuscular) by injection, via, for example, bolus injection or continuous infusion. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents. Alternatively, the active ingredient can be in powder form for constitution with a suitable vehicle, e.g., sterile pyrogen-free water, before use. It is preferred that the TH cell subpopulation cells be introduced into patients via intravenous administration.

The compounds can also be formulated in rectal compositions such as suppositories or retention enemas, e.g., containing conventional suppository bases such as cocoa butter or other glycerides.

In addition to the formulations described previously, the compounds can also be formulated as a depot preparation. Such long acting formulations can be administered by implantation (forexample subcutaneously or intramuscularly) or by intramuscularinjection. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

### 5.8. Vaccine Therapy

Peptides and proteins encoded for by the SGA-1M gene and nucleic acids which encode an SGA-1M polypeptide or fragments thereof, can be used as vaccines by administering to an individual at risk of developing cancer an amount of said protein, peptide, or nucleic acid that effectively stimulates an inunune response against an SGA-1M-encoded polypeptide and protects that individual from cancer. The invention thus contemplates a method of vaccinating a subject against cancer wherein said subject is at risk of developing cancer.

Many methods may be used to introduce the vaccine formulations described above, these include but are not limited to intranasal, intratracheal, oral, intradermal, intramuscular, intraperitoneal, intravenous, and subcutaneous route. Various adjuvants may be used to increase the immunological response, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum. Such adjuvants are also well known in the art.

The nucleotides of the invention, including variants and derivatives, can be used as vaccines, e.g., by genetic immunization. Genetic immunization is particularly advantageous as it stimulates a cytotoxic T-cell response but does not utilize live attenuated vaccines, which can revert to a virulent form and infect the host causing complications from infection. As used herein, genetic immunization comprises inserting the nucleotides of the invention into a host, such that the nucleotides are taken up by cells of the host and the proteins encoded by the nucleotides are translated. These translated proteins are then either secreted or processed by the host cell for presentation to immune cells and an immune reaction is stimulated. Preferably, the immune reaction is a cytotoxic T cell response, however, a humoral response or macrophage stimulation is also useful in preventing initial or additional tumor growth and metastasis or spread of the cancer. The skilled artisan will appreciate that there are various methods for introducing foreign nucleotides into a host animal and subsequently into cells for genetic immunization, for example, by intramuscular injection of about 50 mg of plasmid DNA encoding the proteins of the invention solubilized in 50 ml of sterile saline solution, with a suitable adjuvant (See, e.g., Weiner and Kennedy, 1999, Scientific American 7:50-57; Lowrie et al., 1999, Nature 400:269-271).

The invention thus provides a vaccine formulation for the prevention of cancer comprising an immunogenic amount of an SGA-1M gene product. The invention further provides for an immunogenic composition comprising a purified SGA-1M gene product.

### 5.9. Kits

The invention includes a kit for assessing the presence of cancer cells including breast cancer cells, ovarian cancer cells, skin cancer cells, cancerous cells of the lymphoid system, thyroid cancer cells, pancreatic cancer cells, stomach cancer cells, or lung cancer cells (e.g., in a sample such as a patient sample). The kit comprises a plurality of reagents, each of which is capable of binding specifically with a nucleic acid or polypeptide corresponding to a marker of the invention, e.g., the SGA-1M gene or gene product or fragment thereof Suitable reagents for binding with a polypeptide corresponding to a marker of the invention include antibodies, antibody derivatives, labeled antibodies, antibody fragments, and the like. Suitable reagents for binding with a nucleic acid (e.g., a genomic DNA, an mRNA, a spliced mRNA, a cDNA, or the like) include complementary nucleic acids. For example, the nucleic acid reagents may include oligonucleotides (labeled or non-labeled) fixed to a substrate, labeled oligonucleotides not bound with a substrate, pairs of PCR primers, molecular beacon probes, and the like.

The kit of the invention may optionally comprise additional components usefil for performing the methods of the invention. By way of example, the kit may comprise fluids (e.g., SSC buffer) suitable for annealing complementary nucleic acids or for binding an antibody with a protein with which it specifically binds, one or more sample compartments, an instructional material which describes performance of a method of the invention, a sample of normal cells, a sample of cancer cells, and the like.

### 6. EXAMPLES

The isolation of an uncharacterized breast cancer-associated antigen SGA-1M (Seattle Genetics Antigen isolated from MCF-7 cells) is described. MCF-7 is an estrogen receptor positive (ER+) breast adenocarcinoma cell-line. Suppression Subtractive Hybridization (SSH) and high-throughput cDNA microassays were combined in analyzing genes over-expressed in breast cancer. The results detail the effectiveness ofcombining SSH and cDNA microassays in providing breast cancer-specific expression profiles. Sequence analysis revealed several previously identified cancer-specific genes and additional uncharacterized molecules, including SGA-1M. A full-length cDNA for SGA-1M was isolated using conventional Rapid Amplification ofcDNA Ends (RACE) and cDNA library cloning methods. SGA-1M was determined to be cancer-selective by expression array, northern analysis, semi-quantitative Reverse Transcriptase PCR (RT-PCR), Fluorescence Activated Cell Sorting (FACS), and Immunohistochemistry (IHC). SGA-1M also displayed breast tumor selectivity in a Cancer Profiling Array (CPA). This array showed elevated SGA-1M expression in 22/50 (44%) of the breast cancer patients analyzed using a 2-fold cutoff. SGA-1M polyclonal peptide antibodies were produced against hydrophilic regions and used to confirm selective tumor reactivity by IHC on breast sections containing both primary breast tumor and adjacent normal tissue. SGA-1M expression was also detected in ovarian cancer, skin cancer, a cancer of the lymphoid system, thyroid cancer, pancreatic cancer, stomach cancer, or lung cancer. SGA-1M, based on its tumor selectivity, can be useful as a potential therapeutic target or diagnostic marker in the treatment of breast cancer and other SGA-1M positive cancers.

### 6.1. Introduction

Breast cancer arises from a malignancy of epithelial cells in the female, and occasionally the male, usually of adenocarcinoma origin initiated in the ductal breast epithelium. The majority of breast cancer cases are estrogen-dependent adenocarcinomas. The MCF-7 breast cancer-derived tumor cell line is an estrogen-dependent example. Breast Cancer is the most common non-dermal malignancy in women and 192,200 cases are anticipated in the U.S. for the upcoming year. Despite recent advances in early diagnosis and treatment, 40,200 U.S. women have succumbed to this disease in the year 2000 (Greenlee et al., 2001, Cancer Statistics 51(1):15). Breast cancer, second only to lung cancer in mortality rates annually, requires continued discovery of additional uncharacterized antigens and innovative utility of these molecules to improve overall therapy and intervention.

In total, 10% of all breast cancers are initiated by a genetic mutation similar to BRCA-1 and BRCA-2 (Nathanson et al., 2001, Nature Med. 7(5):552). The transformation of normal epithelium and progression to metastatic breast cancer arises from a cascade of genetic alterations which translate to global changes in cellular protein composition and expression. Some of these changes, detected in the form of cell-surface markers, comprise important diagnostic and tumor targeting efforts being studied currently. For example, the HER-2/neu oncogene, which encodes a 185-kDa protein transmembrane protein, is overexpressed in 10-30% of invasive breast cancers, 40-60% of intraductal breast carcinomas, as well as other cancer types (Koeppen et al., 2001, Histopathology 38(2):96). Antibodies to HER2-neu (Herceptin®) have been shown to identify and selectively sensitize antigen positive cells to anti-cancer therapy (Baselga et al., 1998, Cancer Res. 58:2825).

The sex steroid estrogen has been shown to play a major role in tissue development as well as other physiological processes. In addition, it has been reported to play a critical role in the progression of both breast and gynecological cancers (Pike et al., 1993, Epidemiol. Rev. 15:17). MCF-7 is a well-established tumor cell-line which is an ER+ adenocarcinoma. Despite its existence in cell-culture for nearly three decades, it remains likely that many durable alterations in gene expression patterns still persist since its isolation and initial characterization in 1973 (Brooks et al., 1973, J. Biol. Chem. 248(17):625 1). Some of the above mentioned stabile genes, and specifically SGA-1 M, could provide potential targets for diagnostic or therapeutic strategies for breast cancer. To evaluate this hypothesis, tumor-enriched SSH libraries were constructed and arrayed to selectively screen for tumor-specific genes. SSH is a technique well known in the art for its effectiveness in characterizing and prioritizing differentially expressed genes: (Chu et al., 1997, Proc. Natl. Acad. Sci. 94(19):10057; Gurskaya et al., 1996, Anal. Biochem. 240:90; Kuang et al., 1998, Nuc. Acid Res. 26:1116; von Stein et al., 1997, Nuc. Acid Res. 25:2598; Wong et al., 1997, J. Biol. Chem. 272(40):25190; and Yokomizo et al., 1997, Nature 387:620). SGA-1M, an uncharacterized breast cancer-associated protein, was discovered utilizing these techniques. The initial tumor-enriched MCF-7-specific SSH libraries were evaluated in a higher density format with minimal redundancy, demonstrating that the overall complexity of the libraries had not been compromised.

Intensive and systematic evaluation of gene expression patterns is crucial in understanding the physiological mechanisms associated with cellular transformation and metastasis. Currently, several technical platforms are being used to accomplish this goal. They include: Serial Analysis of Gene Expression (SAGE) (Velculescu et al., 1995, Science 270:484), Restriction Enzyme Analysis of Differentially Expressed Sequences (READS) (Prasher et al., 1999, Methods Enzymol. 303:258), Amplified Fragment Length Polymorphism (AFLP) (Bachem et al., 1996, Plant J. 9:745), Representational Difference Analysis (RDA) (Hubank. et al., 1994, Nucleic Acid Res. 22(25):5640), Differential Display (Liang et al., 1992, Cancer Res. 52(24):6966) and SSH (Diatchenko et al., 1996, Proc. Natl. Acad Sci. 93:6025) as detailed in this text. SSH is very similar to RDA with the exception of an additional normalization step that is included to increase the relative abundance of rare transcripts. The combination of SSH and cDNA microarrays offers several advantages vs. the aforementioned technologies in the discovery of novel tumor-associated proteins and antigens (TAA's). The use of SSH is an attractive approach to identifying novel cancer targets because it does not rely on previously characterized cDNA sets. SSH efficiently normalizes both frequent and rare transcripts at equivalent levels and preferentially amplifies only those which are differentially expressed. The use of expression arrays further increases the chances of identifying lead targets by examining thousands of genes in a single experiment.

### 6.2. Materials and Methods

### 6.2.1. Cell Culture

Breast tumor cell-lines MCF-7, SKBR-3, MDA-MB-231, MDA-MBA35S, Hs578T and BT-549 (ATCC, Manasas, Va.) were grown in RPMl 1640 medium® supplemented with 10% fetal bovine sernm plus 100 U/mL penicillin G and 100 μg/mL streptomycin sulfate. All tumor cell-lines were passaged once per week by trypsinization: replated at 2500-3000 cells/cm². Normal human mammary epithelial cells (HMEC) were maintained in MEGM® (Clonetics, San Diego, Calif.). HMEC's were passaged once per week by trypsinization and replating at 2500-3000 cells/cm².

### 6.2.2. RNA Isolation

Total RNA was isolated from cultured cells using RNAzol B® (Tel-Test, Inc., Friendswood, Tex.). Poly A+ RNA was extracted using the Oligotex mRNA Midi kit ®(Qiagen, Inc., Valencia, Calif.).

### 6.2.3. Generation of Tumor-Enriched SSH cDNA Libraries

Two MCF-7 breast cancer-specific SSH cDNA libraries were constructed essentially as described by Diatchenko et al., 1996, Proc. Natl. Acad Sci. 93:6025, using a PCR-Select™ kit (BD Biosciences-Clontech, Palo Alto, Calif.) with modifications. Library one was constructed using the breast tumor ER+ cell-line MCF-7 (tester) vs. HMEC (driver). Library two was constructed using the breast tumor ER+cell-line MCF-7 (tester) vs. a pool of 5 ER− cell lines (SKBR-3, MDA-MB-231, MDA-MB-435s, Hs578T, and BT-549) (driver).

Driver cDNA was synthesized from 2 ug of poly A+ RNA using 1 ul of 10 uM cDNA synthesis primer 5′-TTTTGTACAAGCTT₃₀NIN-3′ (SEQ ID NO:6) and 1 ul of 200 u/ul Superscript II Reverse Transcriptase® (Invitrogen, Carlsbad, Calif.). The resulting cDNA pellet was digested with 1.5 ul of 10 u/ul of Rsa I restriction enzyme. Driver cDNA's were then precipitated with 100 ul of 10M Ammonium Acetate (Sigma, St. Louis, Mo.), 3 ul of 20 mg/ml glycogen (Roche Molecular Biochemicals, Indianapolis, Ind.) and 1 ml of ethanol (Sigma, St. Louis, Mo.). The cDNA preparations were then resuspended in 5 ul of diethyl pyrocarbonate (DEPC) treated water.

Tester cDNA was synthesized from 2 ug of poly A+ RNA as described above for the driver. Rsa I digested tester cDNA was diluted in 5 ul of DEPC treated water prior to adaptor ligation. Diluted tester cDNA (2 ul) was ligated to 2 ul of 10 uM adaptor 1 (5′-CTAATACGACTCACTATAGGGCTCGAGCGGCCGCCCGGGCAGGT-3′) (SEQ ID NO:7) and 2 ul of 10 uM adaptor 2 R (5′-CTAATACGACTCACTATAGGGCAGCGTGGTCGCGGCCGAGGT-3′) (SEQ ID NO:8) in separate reactions using 0.5 units of T4 DNA ligase (Invitrogen, Carlsbad, Calif.).

Driver cDNA (600 ng) was added separately to each of the two tubes containing adaptor-1 ligated tester (20 ng) and adaptor 2R ligated tester (20 ng). The samples were mixed, ethanol precipitated as described above, and resuspended in 1.5 ul of hybridization buffer (50 mM Hepes pH 8.3, 0.5 M NaCl/0.0.2 mM EDTA pH 8.0). The reaction mixture was placed in hot start PCR tubes, (Molecular BioProducts, San Diego, Calif.), denatured at 95° C. for 1.5 min. and then incubated at 68° C. for 8 hrs. After this initial hybridization, the samples were combined and excess heat denatured driver cDNA (150 ng) was added. This secondary reaction mixture was incubated overnight at 68° C. The final hybridization mixture was diluted in 200 ul of dilution buffer (20 mM Hepes pH 8.3, 50 mM NaCl, 0.2 mM EDTA) and stored at −20° C.

Two rounds of PCR amplification were performed for each SSH library. The primary PCR was performed in 25 ul. The reaction mixture contained 1 ul of diluted subtracted cDNA, 1 ul of 10 uM PCR primer 1 (5′-CTAATACGACTCACTATAGGGC-3′) (SEQ ID NO:9), 10× PCR buffer consisting of (166 mM (NH₄)2504, 670 mM Tris pH 8.8, 67 mM MgCl₂, and 100 mM 2-Mercaptoethanol), 1.5 ul of 10 mM dNTP's, 1.5 ul Dimethyl Sulfoxide (DMSO) (Sigma, St. Louis, Mo.), and 0.25 ul of 5 u/ul of Taq polymerase (Brinkrnann, Westbury, N.Y.). PCR was performed with the following cycling conditions:

75° C. for 7 min.; 94° C. for 2 min.; 27 cycles at 94° C. for 30 sec., 66° C. for 30 sec., and 72° C. for 1.5 min.; and a final extension at 72° C. for 5 min. A secondary PCR was performed using 1 ul of the primary PCR as template with the same reaction components as above. Nested PCR primers NP1 (5′-TCGAGCGGCCGCCCGGGCAGGT-3′) (SEQ ID NO:10) and NP2R (5′-AGCGTGGTCGCGGCCGAGGT-3′) (SEQ ID NO: 11) were used in place of PCR primer 1. The secondary PCR was performed with the following cycling conditions: 94° C. for 2 min.; 15 cycles at 94° C. for 30 sec., 68° C. for 30 sec., and 72° C. for 1.5 min.; and a final extension at 72° C. for 5 min. The PCR products were analyzed on 1.5% ultrapure agarose gels (Invitrogen, Carlsbad, Calif.) and visualized by ethidium bromide (Fisher Chemical, Fair Lawn, N.J.).

Subtraction efficiency was confirmed by PCR depletion of EF-1 and Tubulin. EF-1 primers were EF-1 (5′-CTGTTCCTGTTGGCCGAGTC-3′) (SEQ ID NO:12) and EF-2 (5′-CGATGCATTGTTATCATTAAC-3′) (SEQ ID NO:13) corresponding to GenBank# T40408 (Hillier el al., 1995). Tubulin primers were Tul (5′-CACCCTGAGCAGCTCATCAC-3′) (SEQ ID NO:14) and Tu2 (5′-GGCCAGGGTCACATTTCACC-3′) (SEQ ID NO:15) corresponding to GenBank# H22238 (Hillier el al., 1995).

### 6.2.4. Cloning of SSH Tumor-Enriched Pools into PCR4-TOPO

The SSH-cDNA pools were cloned into the pCR4-TOPO® vector (Invitrogen, Carlsbad, Calif.) and transformed into chemically competent TOP 10 cells® (Invitrogen, Carlsbad, Calif.). The library was plated on LB agar plates (Becton Dickinson, Sparks, Md.) containing 50 μg/ul kanamycin (Sigma, St. Louis, Mo.). Cloning efficiency and size distribution for each library was determined by amplification using M13 (−20) (5′-GTAAAACGACGGCCAGT-3′) (SEQ ID NO:16) and M13R (5′-CAGGAAACAGCTATGACC-3′) (SEQ ID NO:17) universal primers.

### 6.2.5. Custom Array Generation

SSH clones containing cDNA sequences of interest were amplified using M13 (−20) (SEQ ID NO:16) and M13R (SEQ ID NO:17) universal primers. PCR products were purified using 96-well MultiScreen PCR Purification Plates (Millipore, Bedford, Mass.). Microarrays were prepared by spotting targets in duplicate on positively charged nylon membranes (Hybond-XL®, Amersham Pharrnacia Biotech, Piscataway, N.J.) at concentrations of 2 ng DNA/spot using a Biomek 2000 Robot® (Beckman Coulter Inc., Fullerton, Calif.). For probe construction, mRNA was isolated from cell lines as described above. Poly A+ RNA (500 ng) was converted to cDNA and labeled with (α-P32) dCTP (Amersham Pharmacia Biotech, Piscataway, N.J.) by reverse transcription using Superscript II RT® (Invitrogen, Carlsbad, Calif.). Hybridizations were performed overnight at 42° C. in 6× Saline Sodium Citrate (SSC), 0.1% Sodium Dodecyl Sulfate (SDS), 50% Deionized Formarnide, and 5× Denhardt's solution (1% Ficoll Type 400, 1% polyvinylpyrrolidone, and 1% bovine serum albumin) (Research Genetics, Huntsville, Alab.). Wash conditions were 4 times in 2×SSC/0.1% SDS for 10 min. each at room temperature, followed by 4 high stringency washes in 0.1×SSC/0.1% SDS at 65° C. for 30 min. each.

### 6.2.6. Array Data Analysis

Hybridization Intensities were quantitated on the Phosphorimager SI® (Molecular Dynamics, Sunnyvale, Calif.) using ArrayVision 5.1 Software® (Imaging Research, St. Catharines, ON, Calif.). An average signal intensity was determined for each set of duplicate spots. For each membrane analyzed, relative quantitative values were determined based on normalization to multiple housekeeping genes spotted at various locations on each membrane. This enabled us to make blot-to-blot comparisons in determining differential expression. Two independent micro array experiments were performed for each comparison to ensure overall validity and reproducibility of the results. Targets >2 fold over-expressed were considered for further evaluation.

### 6.2.7. Construction of an MCF-7 Lambda ZAP-CMV XR CDNA Library

An MCF-7 cDNA library was generated to facilitate full-length cloning of breast cancer candidate genes from 5 μg mRNA using the Lambda ZAP-CMV XR Library Construction Kit® with modifications (Stratagene, La Jolla, Calif.). The disaccharide, trehalose (Sigma, St. Louis, Mo.), was added during the first strand cDNA synthesis to minimize the tendancy for secondary structure formation (Carnici et al., 1999, Methods in Enzymology 303:19). The cDNA was size fractionated using size sep 400 CL-4B® spin columns (Amersham Pharmacia Biotech, Piscataway, N.J.) and directionally cloned into the EcoRI/XhoI predigested Lambda ZAP-CMV XR® vector. Efficiency of cloning, average insert size distribution, and overall library quality was evaluated by PCR using universal primers T7 (5′-TAATACGACTCACT ATAGGG-3′) (SEQ ID NO: 18) and T3 (5′-ATTAACCCTCACTAAAGGGA-3′) (SEQ ID NO:19) and primers specific to the 5′-ends of various genes known to be expressed in MCF-7.

### 6.2.8. Northern Analysis

Aliquots of 1 ug mRNA were resolved on 1.2% agarose formaldehyde gels in 1× 3-N-morpholino propanesulfonic acid (MOPS) buffer (5× stock of 0.1M MOPS (pH 7.0), 40 mM sodium acetate (Sigma, St. Louis, Mo.), and 5 mM EDTA (Ambion, Austin, Tex.)) and then transferred to Hybond-XL Nylon Membranes® (Amersham Pharmacia Biotech, Piscataway, N.J.). Universal primers M13F (−20) (SEQ ID NO:16) and M13R (SEQ ID NO:17) were used to amplify the commercially available clone GenBank# T40408 (Hillier et al., 1995) specific for EF-1 (Incyte Genomics, St. Louis, Mo.). Amplified probes spanning the region from 225-706 bp in SGA-1M, approximately 50 ng, were labeled using Ready-to-go Beads® and α-P32 dCTP at 3000 Ci/mmol (both from Amersham Pharnacia Biotech, Piscataway, N.J.). Northern blots were pre-hybridized and hybridized for 1 hour using ExpressHyb® hybridization solution (Clontech, Palo Alto, Calif.). Blots were washed in 2×SSC and 0.1% SDS for 1 hour at room temperature, followed by an additional hour at 65° C. in 0.1×SSC and 0.1% SDS. Northern blots were quantified using ImageQuant Sottware® (Molecular Dynamics, Sunnyvale, Calif.).

### 6.2.9. Semi-Quantitative RT-PCR

cDNA was synthesized from 100 ng of poly A+ RNA and 5 ug total RNA using the Superscript First-Strand cDNA Synthesis System for RT-PCR® (Invitrogen, Carlsbad, Calif.). Gene specific primers were selected for SGA-1M and EF-1 to obtain semi-quantitative mRNA levels. Primers for SGA-1M were as follows: SGA-1M-1F (5′-GGCTGCAGGTGATGCTCCTCCACC-3′) (SEQ ID NO:20), and SGA-1M-1R (5′-ATCATCCCGACCCACAAAATCCTCATC-3′) (SEQ ID NO:21) spanning the region from 272bp-482 in the 1905 bp SGA-1M cDNA. Primers for EF-1 were as follows: EF-1 (5′-CTGTTCCTGTTGGCCGAGTC-3′) (SEQ ID NO:22) and EF-2 (5′ CGATGCATTGTTATCATTAAC-3′) (SEQ ID NO:23) corresponding to GenBank# T40408 (Hillier et al., 1995).

### 6.2.10. Multiple Tissure Expression Array (MTE)

The MTE® (Clontech, Palo Alto, Calif.) array was used to determine relative expression of SGA-1M in various normal populations. 50 ng of an SGA-1M PCR product spanning 225-706 bp was labeled using Ready-to-go Beads® and α-P32 dCTP at 3000 Ci/mmol. The housekeeping control, EF-1, was used to evaluate the spot-to-spot variability within the experiment.

### 6.2.11. Cancer Profiling Array (CPA)

The CPA® (Clontech, Palo Alto, Calif.) was used to determine the expression of SGA-1M in numerous tumor/normnal paired patient samples. Fifty ng of an SGA-1M PCR product spanning 225-706 bp was labeled using Ready-to-go Beads and α-P32 dCTP at 3000 Ci/mnuol. A total of 241 paired cDNA samples were synthesized and spotted onto nylon membranes for 13 different tumor types. The tumor types included: Breast, Cervix, Colon, Kidney, Lung, Ovarian, Pancreas, Prostate, Rectum, Thyroid Gland, Small Intestine, Stomach, and Uterus.

### 6.2.12. Bioinformatics Analysis

After completion of the array data analysis sorting process, interesting novel targets were retained and analyzed ftirther using several computational programs. SGA-1M was analyzed using Compugen's Proprietary LEADS Software® in association with their Lab on Web service® (Compugen Ltd., Tel-Aviv, Israel). Lab on Web provides a means of facilitating the characterization of novel cDNA sequences.

The derived full-length cDNA for SGA-1M was analyzed using Vector NTI Suite 6.0® (InforMax, Inc., Bethesda, Md.). Transmembrane domain and protein localization analysis was performed using the ExPASy Proteomics Tools Programs® (Swiss Institute of Bioinformatics, Geneve, Switzerland). Amino acid sequence prediction programs used included: HMMTOP (Tusnady et al., 1998, J. Mol. Bio. 283:489), TM pred (Hofinann et al., 1993, J. Biol. Chem. 347:166), TMHMM v1.0 (Sonnhamrner et al., 1998, Proc. of Sixth Int. Conf on Intelligent Systems for Mol. Bio., AAAI Press, pp. 175-182), TMAP, and PSORT (Nakai et al., 1999, Trends Biochem. Sci. 24(1):34).

### 6.2.13. Polyclonal Antibodies Against SGA-1M

Polyclonal antibodies against two peptides derived from SGA-1M were generated. Anti-SGA-1M(1-2) was directed against the peptide KVRKMPETFSNLPRT (SEQ ID NO:28) corresponding to amino acid residues 201-215 (FIG. 10A). Anti-SGA-1M(1-4) was directed against the peptide PGRDEDFVGRDD (SEQ ID NO:28) corresponding to amino acid residues 92-103 (FIG. 10A). Peptides with the above sequences with an additional cysteine residue at their N-terminal end were synthesized, purified by high performance liquid chromatography, and then lyophilized (Bethyl Labs, Montgomery, Tex.). The peptides were separately conjugated to Keyhole Limpet Hemocyanin (KLH) as carrier, using maleimide chemistry, linking the sulfhydryl of the peptide to the carrier. Each of the isolated preps was injected into separate animals as immunogen. Immunosorbents were prepared by linking SGA-1M peptides to agarose using cyanogen bromide. Affinity purification was determined using hyperimmune serum from rabbits immunized with KLH-SGA-1M peptides and then processed using appropriate immunosorbents to capture antibodies specific for each SGA-1M peptide. Overall potency and performance was evaluated by Enzyme-Linked Immunoadsorbent Assay (ELISA). SGA-1M peptides were coated on microtiter plates, reacted with dilutions of antibody, then with Goat anti-rabbit IgG/Horse Radish Peroxidase substrate.

### 6.2.14. Expression of SGA-1M and Specificity of Anti-SGA-1M Antibodies

An SGA-1M/Myc-His fusion protein construct was generated to determine the specificity of the anti-SGA-1M antisera. A cDNA consisting of the complete coding sequence of SGA-1M was amplified by PCR using an SGA-1M cDNA clone as template. Polymerase chain reaction (PCR) forward primer (5′-GATCGAAAGCTTGCCACCATGGCGTTGGCGTTG GCGGCGCTG-3′) (SEQ ID NO:24) , and reverse primer (5′-GATCGAGAATTCATAAATAAAGAG AACTCTGGTCCTGGG-3′) (SEQ ID NO:25) were synthesized (Sigma Genosys, St. Louis, Mo.) and included a HindIII restriction site (underlined) and a EcoRI restriction site (underlined) in the forward and reverse primers, respectively. The PCR product was cut with the above restriction enzymes and cloned in-frame into HindIII/EcoRI-cut pcDNA4MycHisA (Invitrogen, Carlsbad, Calif.). Expression of this plasmid in eukaryotic cells resulted in the synthesis of an SGA-1M/Myc-His fusion protein. This construct was transiently expressed in COS-7 cells using DEAE-dextran (Sigma, St. Louis, Mo.). Tranfected cells were incubated for 72 hours prior to harvesting. Harvested cells were lysed in a buffer containing 150 mM NaCl, 50 mM Tris, pH 8.0, 5 mM EDTA, 0.5% NP-40, and 2 mM PMSF. Cell lysates were then incubated with anti-Myc (Santa Cruz, San Diego, Calif.), anti-SGA-1M(1-2), or anti-SGA-1M(1-4) to immunoprecipitate the SGA-1M/Myc-His fusion protein. Normal rabbit IgG and normal mouse IgG were used as negative controls. Immunoprecipitates were resolved by non-reducing Tris-glycine polyacrylamide gels, and proteins were transferred onto PVDF membranes (Invitrogen, Carlsbad, Calif.). Membranes were blocked with 5% BSA in Tris-buffered saline containing 0.5% Tween 20 (TBST) (Sigma, St. Louis, Mo.) before immunoblotted with either anti-Myc or anti-SGA-1M(1-4). Protein bands were visualized using horseradish peroxidase conjugated goat anti-mouse IgG or goat anti-rabbit IgG (Jackson IrrununoResearch Laboratories, West Grove, Pa.) and DAB (Vector Labs, Burlingame, Calif.).

### 6.2.15. Immunohistochemistry

Immunohistochemistry (IHC) was performed using breast tumor tissue isolates and adjacent normal sections (Biogenix, San Ramon, Calif.) and multi-tumor tissue grids (Biogenix, San Ramon, CA; Biomeda, Foster City, Calif.). Tissue sections were cleared in Histoclear Solution (National Diagnostics, Atlanta, Ga.) and rehydrated in graded ethanol (100%-70%) (Sigma, St. Louis, Mo.). Endogenous peroxidase activity was quenched by incubation with 0.3% Hydrogen Peroxide/Methanol (Sigma, St. Louis, Mo.). Non-specific binding was blocked by incubation with 10% normal goat serum (Jackson Labs, Bar Harbor, Me.). Primary antibody, either anti-SGA-1M (1-2) or anti-SGA-1M (1-4), was applied at 10 ug/ml in 5% Bovine Serum Albumin (BSA) (Sigma, St. Louis, Mo.). For peptide blocking experiments, SGA-1M peptide antibodies were incubated with corresponding peptides at a 10:1 (peptide:antibody) ratio for 1 hour. Biotinylated goat anti-rabbit IgG (Jackson Labs, Bar Harbor, Me.) secondary antibody was applied at 10 ug/ml in 5% BSA (Sigma, St. Louis, Mo.). Sections were then incubated with an avidin/biotinylated enzyme complex (Vectastain ABC Elite Kit, Vector Labs, Burlingame, Calif.). Slides were developed using a 3,3-Diaminobenzidine (DAB) substrate (Vector Labs, Burlingame, Calif.) and a methyl green counterstain (Vector Labs, Burlingame, Calif.).

### 6.2.16. Subcellular Localization of SGA-1M

The subcellular localization of SGA-1M was determined using an SGA-1M/green fluorescence protein (GFP) fusion protein construct. A cDNA consisting of the complete coding sequence of SGA-1M was amplified by PCR using a full length SGA-1M cDNA clone as template, and the forward primer ( 5 ′-GATCGAGCTAGCGCCACCATGGCGTTGGCGTTGGCGGCGCTG-3′) (SEQ ID NO:26),and the reverse primer (5′-GATCGAAAGCTTATAAATAAAGAGAACTCTGGTCCTGGG-3′) (SEQ ID NO:27). An NheI restriction site (underlined) and a HindIII restriction site (underlined) are present in the forward and reverse primers, respectively. The PCR product was cut with the above restriction enzymes and cloned in-frame into NheI/HindIII-cut pGFP²-N3 (BioSignal Packard, Montréal, Canada). Expression of this plasmid in eukaryotic cells resulted in the synthesis of an SGA-1M/GFP fusion protein. This construct was transiently transfected into COS-7 and Vero cells by electroporation. The subcellular localization of green fluorescence signals, which indicate the localization of the SGA-1M/GFP protein, was monitored by fluorescence microscopy.

### 6.3. Results

### 6.3.1. MCF-7 VS. HMEC Expression Array

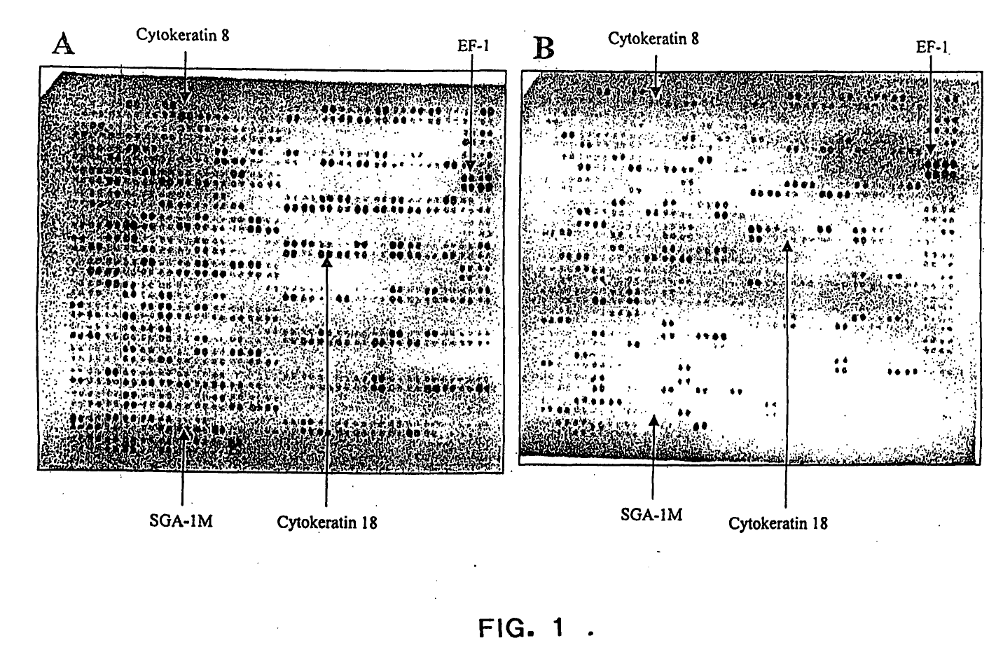

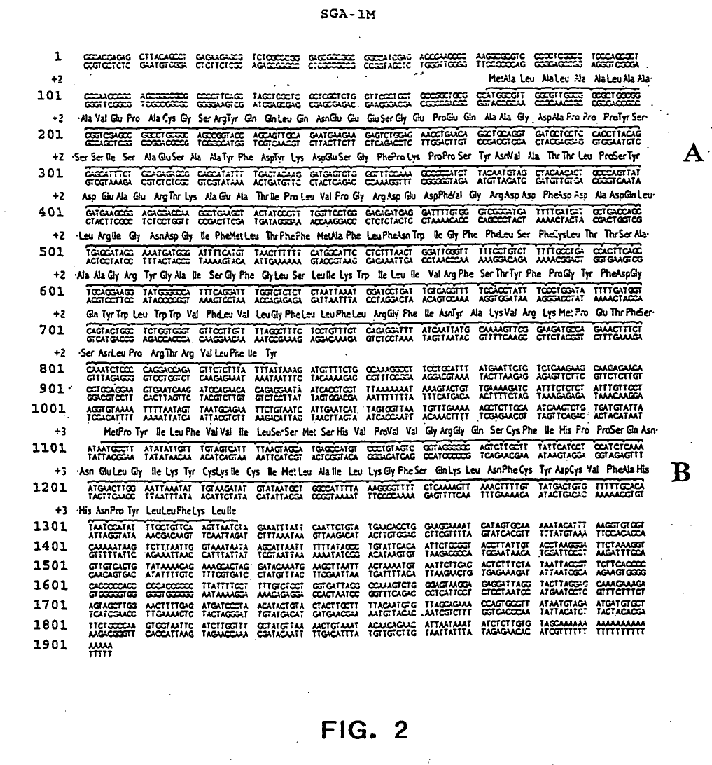

To enrich for genes preferentially expressed in the ER+ breast cancer cell line MCF-7, two separate SSH libraries were generated as described previously (Diatchenko et al. supra). Initially, a total of 576 SSH clones (288 from each library) were evaluated in duplicate by microarray experiments format (FIG. 1). Initial sequencing results revealed several known genes previously implicated in breast cancer as well as the uncharacterized protein, SGA-1M. As previously reported in the literature, breast cancer-specific markers cytokeratin 8 (GenBank# X12882)(Franke et al. 1996) and cytokeratin 18 (GenBank# M26326) (Oshima et al. 1995) were found to be significantly over-expressed in our MCF-7 vs. HMEC comparison (FIG. 1) (Trask et al., 1990, Proc. Natl. Acad. Sci. 87(6):2319). Hybridization intensities were normalized and quantified on a Phosphorimager SI® (Molecular Dynamics, Sunnyvale, Calif.) and analyzed using ArrayVision 5.1 Software® (Imaging Research, St. Catharines, ON, Calif.). Duplicate hybridizations were performed to validate quantitative predictions. Quantitative values of over-expression in the MCF-7 vs. HMEC comparison for cytokeratin 8, cytokeratin 18, and SGA-1M were: 11-fold, 6-fold, and 5-fold, respectively (FIG. 1).

### 6.3.2. Full-Length Cloning of SGA-1M cDNA

### 6.3.2.1. RACE-PCR

The original SGA-1M SSH clone (FIG. 1), as spotted on the microarray, spanned the region within the full-length cDNA from 225-706 bp. The Swithing Mechanism at 5′ ends of RNA Transcripts (SMART) RACE® cDNA amplification kit (Clontech, Palo Alto, Calif.) was used to extend the cDNA sequence for SGA-1M from 249-1905 bp (Matz et al., 1999, Nuc. Acids. Res. 27:1558). SGA-1M specific PCRprimers were used to amplifyregions ofinterestand were cloned into the TOPO TA pCR 4.0® vector (Invitrogen, Carlsbad, Calif.).

### 6.3.2.2. Lambda ZAP cDNA Library

The MCF-7 cDNA library was used to isolate the 5′-most portion of the full-length SGA-1M cDNA spanning 1-248 bp. The nucleotide region of SGA-1M from 249-1905 was isolated by RACE-PCR methods as previously described. The 3′ RACE product and the 5′ library clone were combined to form the full-length CDNA of 1905 bp for SGA-1M (FIG. 2). The complete full-length CDNA sequence for SGA-1M was determined by automated fluorescent sequencing (PE Applied Biosystems, Foster City, Calif.) using custom primers (Sigma-Genosys, Woodlands, Tex.).

### 6.3.3. Cancer Selectivity by Northerns, RT-PCR, and Commercial Arrays

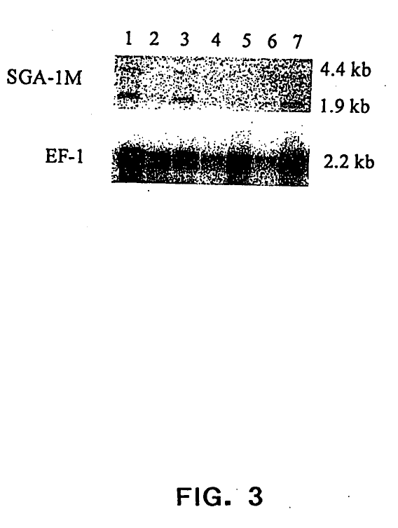

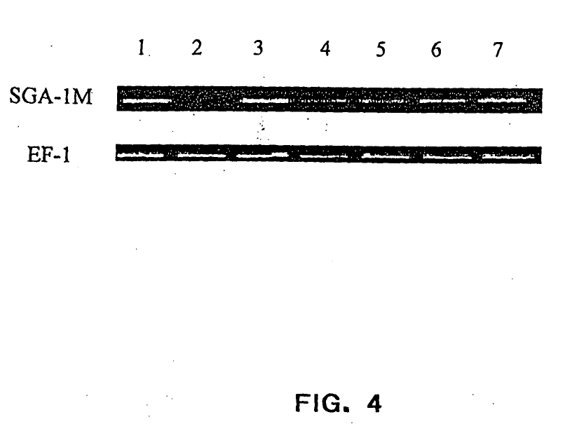

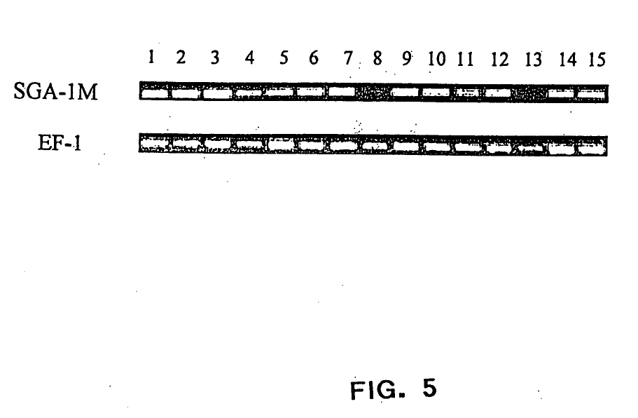

SGA-1M was initially determined to be over-expressed by both microarray and northern analysis in an MCF-7 vs. HMEC comparison (FIG. 1 and FIG. 3). After further evaluation, SGA-1M displayed preferential expression in MCF-7 (ER+ Breast Adenocarcinoma), SKBR-3 (ER− Breast Adenocarcinoma), MDA-MB23 1 (ER− Breast Adenocarcinoma), MDA-MB4355 (ER− Breast Ductal Carcinoma), Hs578T (ER− Breast Adenocarcinoma), and BT549 (ER− Breast Adenocarcinoma) tumor cell-lines (ATCC, Manasas, Va.) while exhibiting minimal expression in normal HMEC's (FIGS. 3 and 4). SGA-1M was also significantly expressed in other ATCC tumor cell-lines, including: WM266-4 (Metastatic Melanoma), NIH:OVCAR3 (Ovarian Adenocarcinoma), SKOV3 (Ovarian Adenocarcinoma), PA-1 (Ovarian Teratocarcinoma), Raji (Burkitt's Lymphoma), and Ramos (Burkitt's Lymphoma) as detected by semi-quantitative RT-PCR (FIG. 5). Significant levels of SGA-1M expression were determined based on comparative analysis using the housekeeping gene EF-1 (19) as a reference standard (FIGS. 4 and 5).

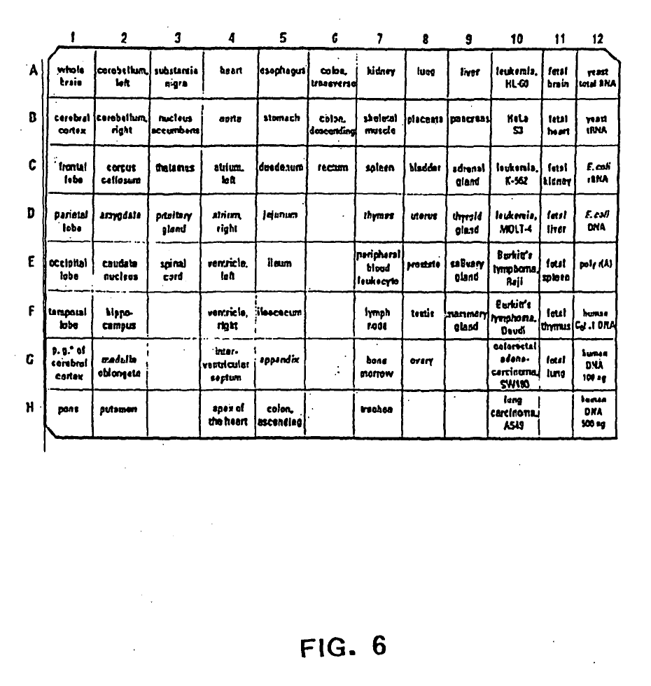

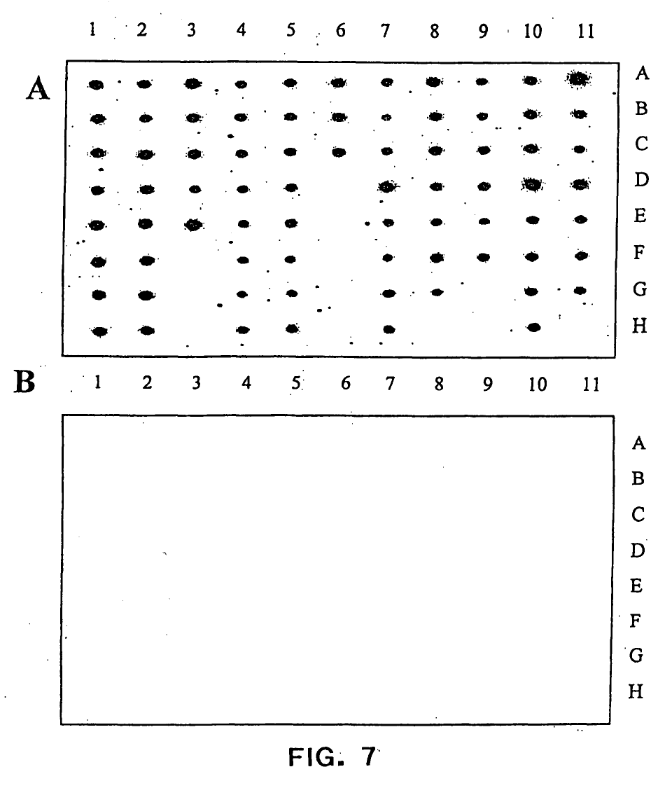

To confirm minimal normal tissue expression of SGA-1M, the MTE Array (Clontech, Palo Alto, Calif.) was hybridized using a SGA-1M-specific probe from 225-706 bp. The SGA-1M transcript was minimally expressed and limited to a few of the normal tissues tested (FIGS. 6 and 7). Of particular interest was the minimal degree of expression observed in both breast and ovary normal isolates (FIG. 7). As detailed above, SGA-1M exhibited significant levels of breast and ovarian tumor expression in multiple cell-lines by semi-quantitative RT-PCR (FIG. 5). Overall, SGA-1M is significantly expressed in breast cancer, ovarian cancer, and melanoma (FIG. 5).

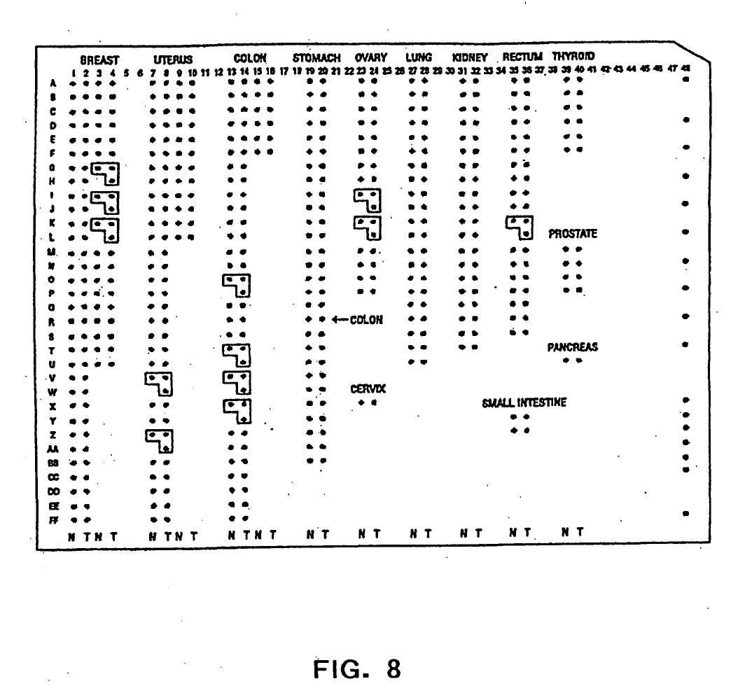

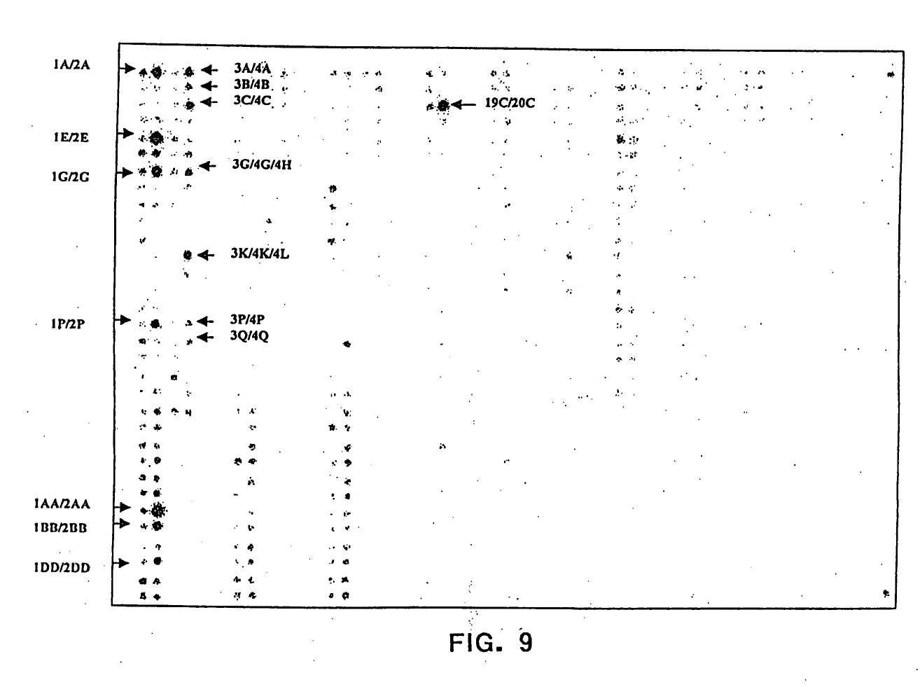

To confirm SGA-1M over-expression in patient tumor isolates, the CPA was hybridized using a SGA-1M-specific probe from 225-706 bp of the full-length cDNA. In total, 50 paired breast tumor/normal isolates were analyzed (FIGS. 8 and 9). The array results displayed SGA-1M over-expression in 22/50 (44%) of breast tumor isolates using a 2-fold differential (Table 3). SGA-1M was also over-expressed in 11/50 (22%) at a 5-fold differential, and in 8/50 (16%) at a 10-fold differential (Table 3). SGA-1M also displayed high differentials in breast cancer isolates reported to have lymph node metastases at positions 2H (2-fold), 2N (13-fold), 2P (11-fold), 4P (3-fold), 2D (4-fold), and 2E (28-fold) (Table 3). Based on its high percentage of differential expression (44% of the breast samples tested), SGA-1M is useful as a diagnostic marker for breast cancer progression to metastasis.

TABLE 3
Table 3. SGA-1M breast cancer over-expression on the Cancer Profiling
Array (FIG. 9). A total of fifty breast patient isolates were
analyzed in this experiment. Twenty-two of fifty paired isolates
displayed >2-fold over-expression in a tumor vs. normal quantitative
comparison as listed in the T/N column.

| Tissue Source | Array Position | T/N |
|---|---|---|
| Non-infiltrating Intraductal Carcinoma (n = 1) | 1A/2A | 7 |
| Infiltrating Ductal Carcinoma (n = 14) | 3A/4A | 22 |
| | 3B/4B | 4 |
| | 3F/4F | 4 |
| | 1G/2G | 7 |
| | 3G/4G/4H | 3 |
| | 1H/2H (met.) | 2 |
| | 3K/4K/4L | 17 |
| | 3M/4M | 4 |
| | 1N/2N (met.) | 13 |
| | 1P/2P (met.) | 11 |
| | 3P/4P (met.) | 3 |
| | 1U/2U | 2 |
| | 1Z/2Z | 3 |
| | 1AA/2AA | 17 |
| Tubular Adenocarcinoma (n = 1) | 3C/4C | 9 |
| Infiltrating Intraductal Carcinoma (n = 2) | 1D/2D (met.) | 4 |
| | 1E/2E (met.) | 28 |
| Medullary Carcinoma (n = 1) | 3Q/4Q | 12 |
| Fibrosarcoma (n = 1) | 1X/2X | 2 |
| Mixed Lobular Carcinoma (n = 1) | 1BB/2BB | 11 |
| Infiltrating Lobular Carcinoma (n = 1) | 1DD/2DD | 6 |

### 6.3.4. Expression of the SGA-1M Protein in Tumors

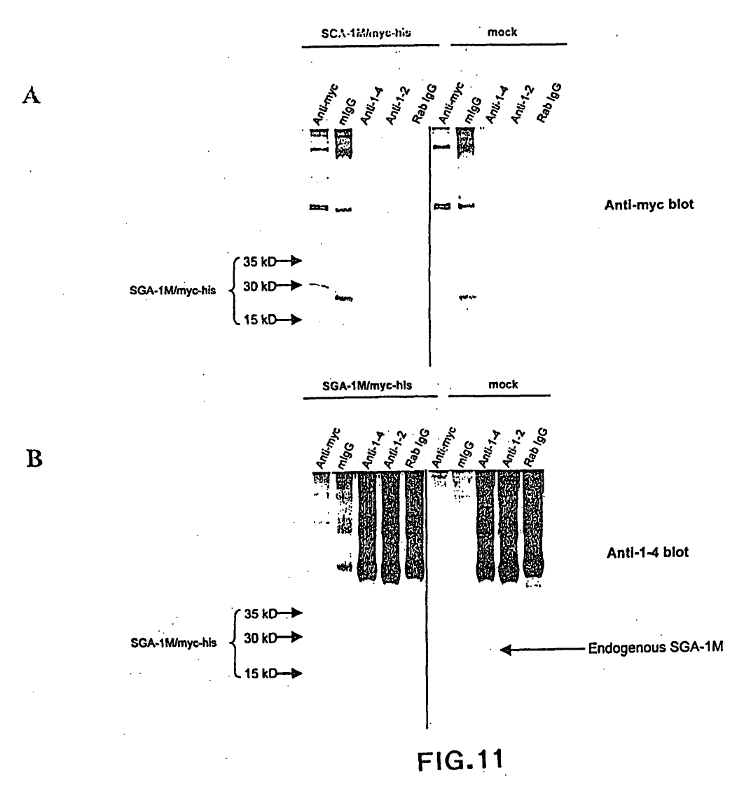

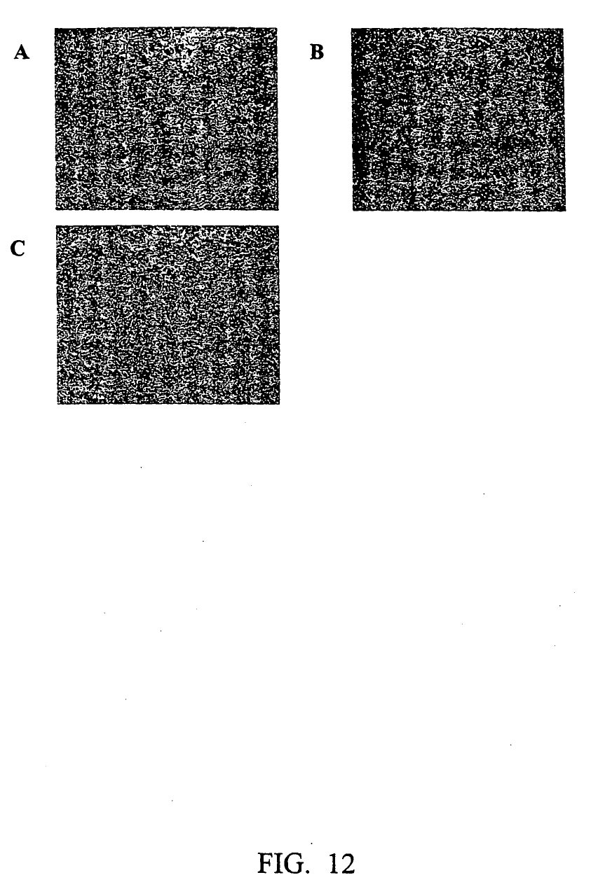

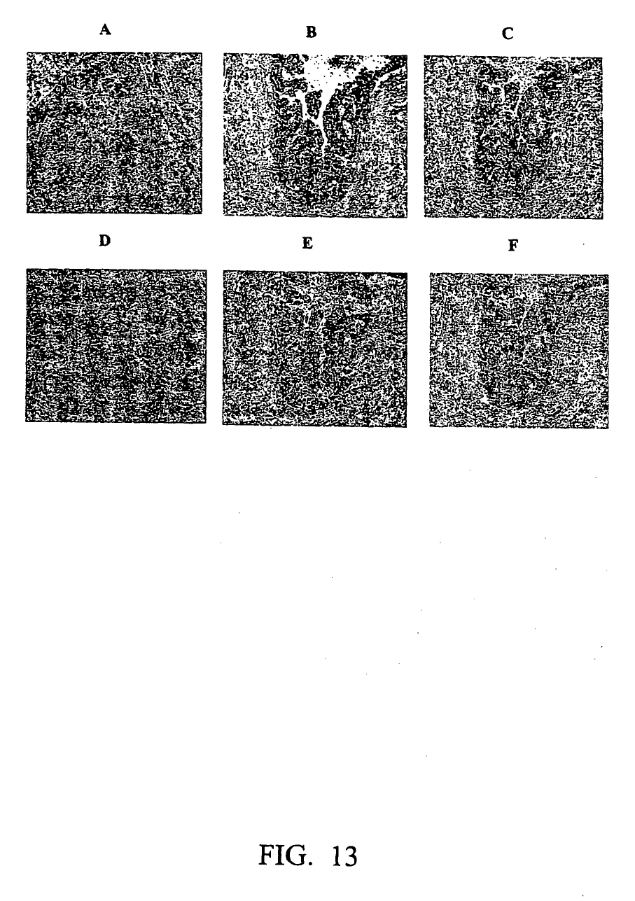

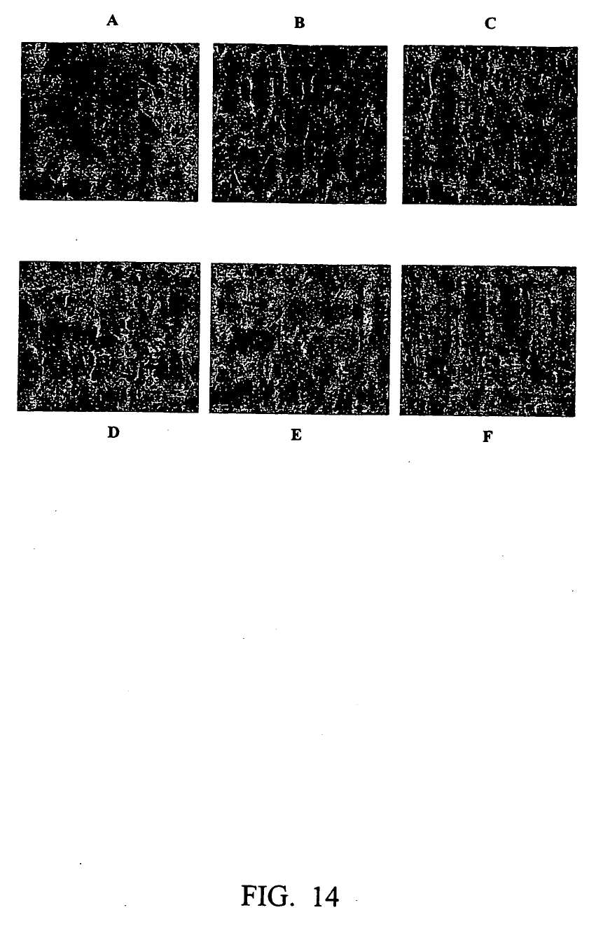

Antibodies against SGA-1M were generated in order to examine the SGA-1M protein expression in normal and breast carcinoma tissues according to procedures described in 6.2.13. The specificity of anti-SGA-1M (1-2) and anti-SGA-1M (1-4) was determined by methods described in section 6.2.14. FIG. 11 shows that the anti-SGA-1M antibodies recognized both the SGA-1M/yc-His fusion protein and endogenous SGA-1M are expressed in COS-7 cells. Immunohistochemistry (IHC) was performed on paraffin embedded tissue sections using SGA-1M (1-2) and anti-SGA-1M (1-4) rabbit polyclonal antibodies (FIG. 12 and FIG. 13). Tumor-selective staining was observed using either anti-SGA-1M (1-2) or anti-SGA-1M (1-4) antibodies (FIG. 13). The well-characterized breast cancer-specific antibody BR96 (Helistrom et al., 1990, Cancer Res. 50(7):2183) was used as a positive control to confirm tumor selectivity (FIG. 12). Reactivity of both anti-SGA-1M (1-2) and anti-SGA-1M (1-4) was blocked with the immunizing peptides by pre-incubation at ratios of 10:1 (peptide:antibody) (FIG. 13). Tumor specific IHC staining using anti-SGA-1M (1-4) was observed in multiple tumor types, including: breast adenocarcinoma, melanoma, thyroid carcinoma, lymphoma, pancreatic adenocarcinoma, and stomach adenocarcinoma (FIG. 14).

### 6.3.5. Subcellular Localization of SGA-1M

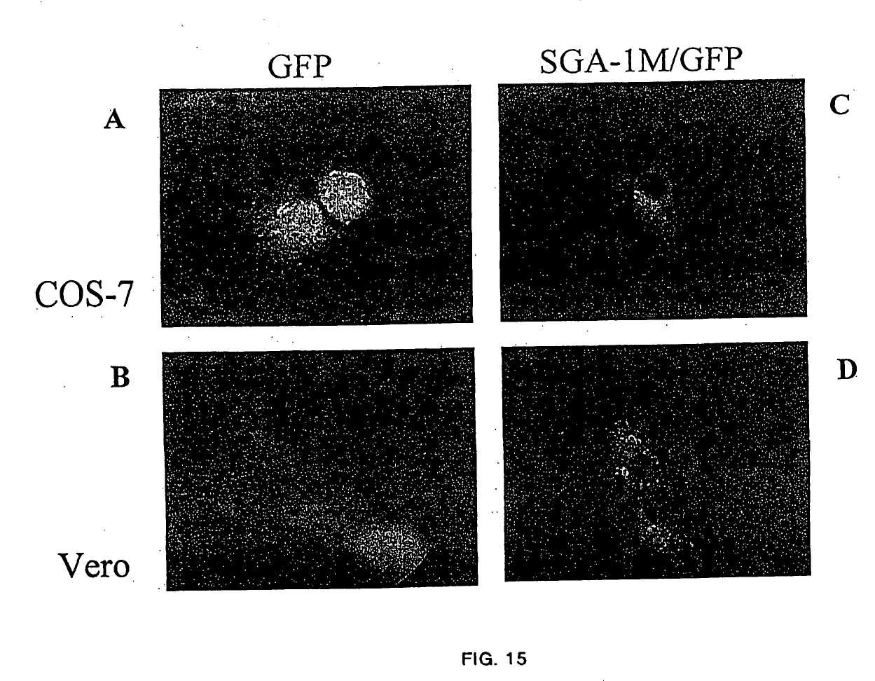

Subcellular localization of SGA-1M was determined using transient expression of SGA-1M/GFP constructs in both COS-7 and Vero cells. Expression of GFP alone resulted in diffused green fluorescence signals throughout the cells (FIG. 15A and 15B). On the other hand, expression of SGA-1M/GFP resulted in fluorescence signals localized mostly outside the nuclei of cells in the perinuclear region and in the form of vesicles (FIG. 15C and 15D). Such a pattern indicates that SGA-1M/GFP most likely localizes to the endoplasmic reticulum (ER) and Golgi apparatus, as previously reported (Simpson et al., 2000, EMBO Reports 1(3):287-292). The ER and Golgi apparatus constitute part of the cellular protein secretory and plasma membrane biogenesis pathway. SGA-1M, containing multiple putative transmembrane sequences, is potentially associated with expression on cells as an integral plasma membrane protein.

### 6.3.6 Comparison of SGA-1M with Genbank Sequences

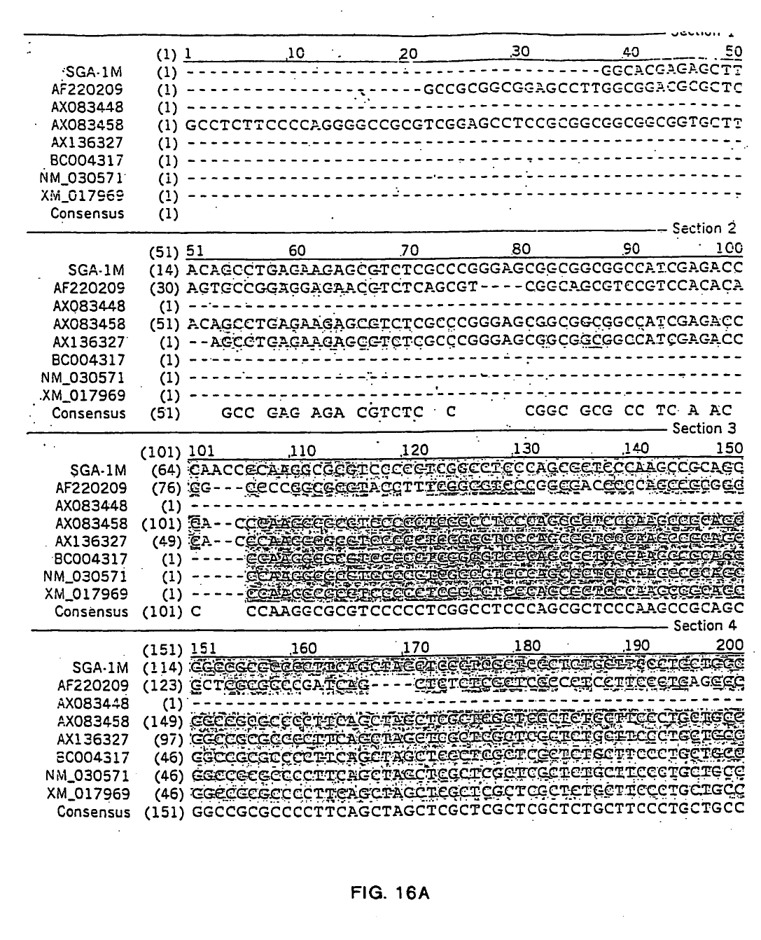

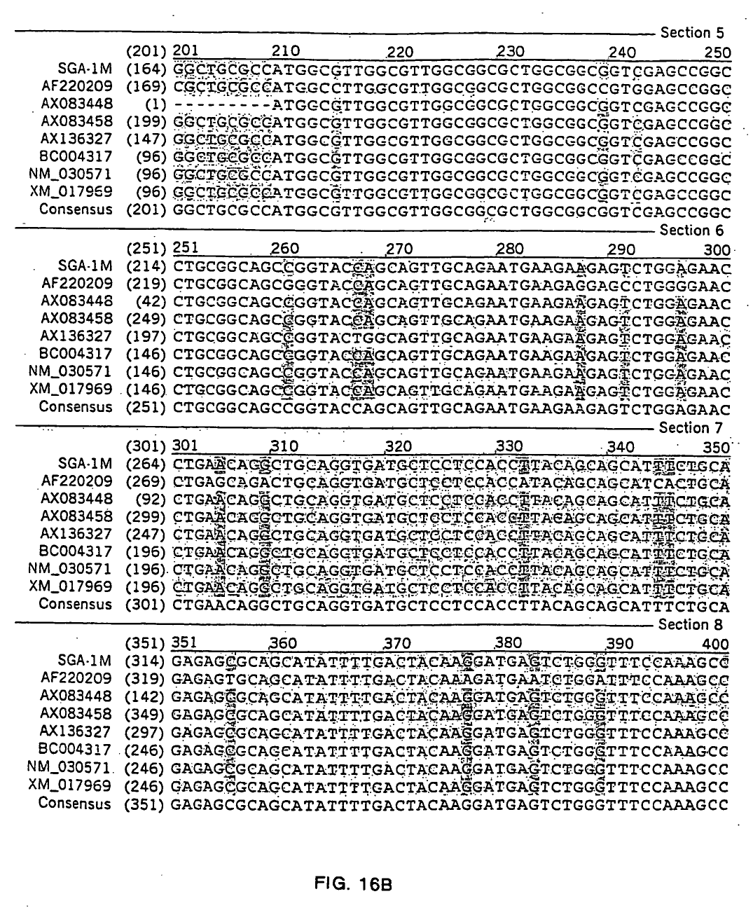

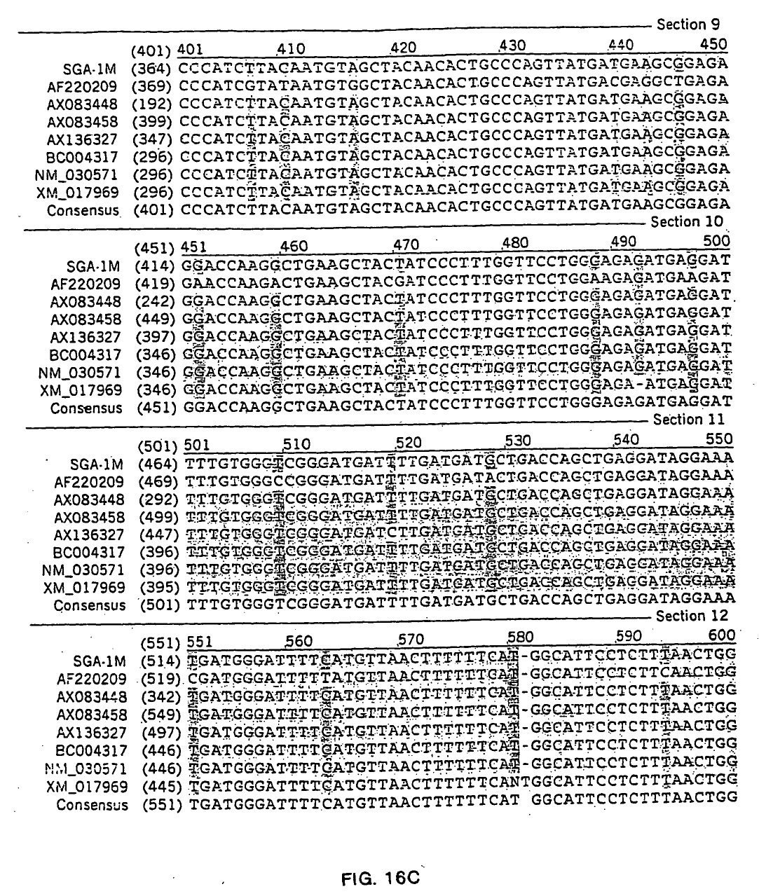

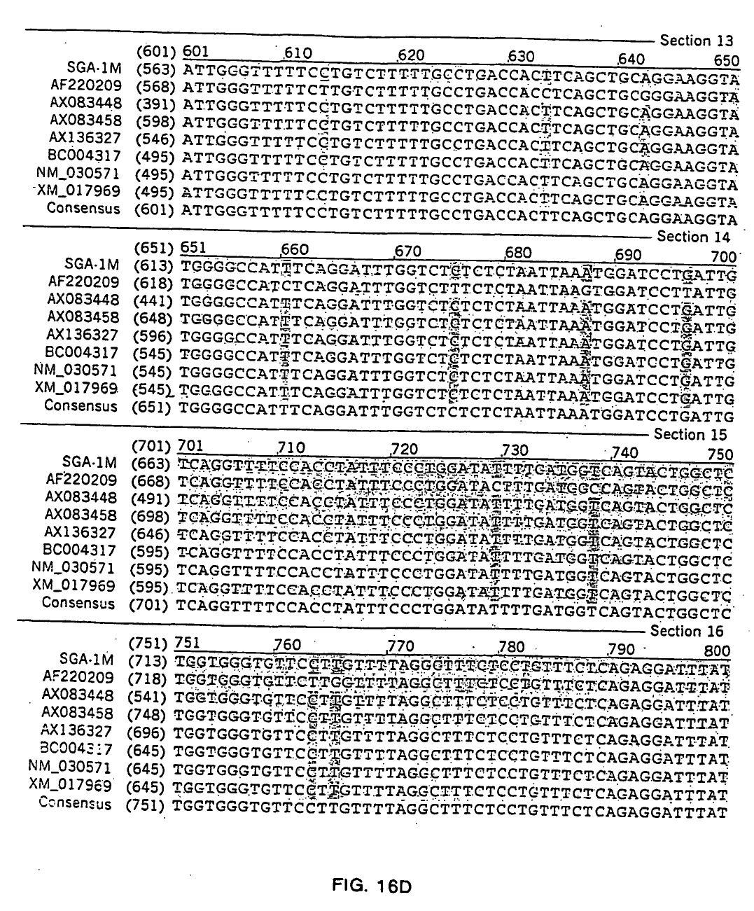

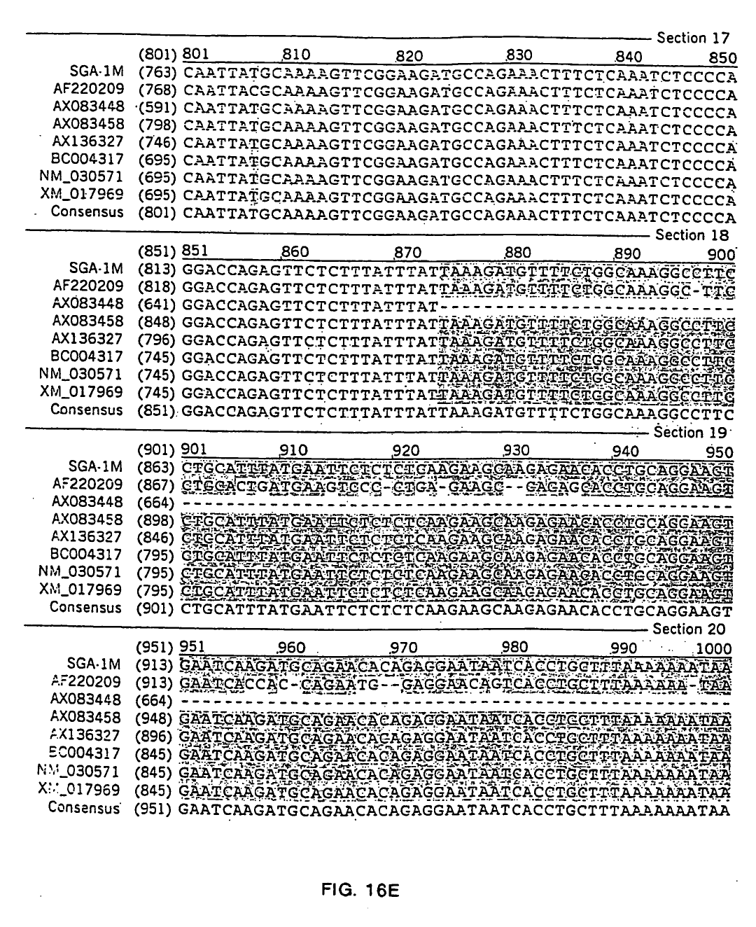

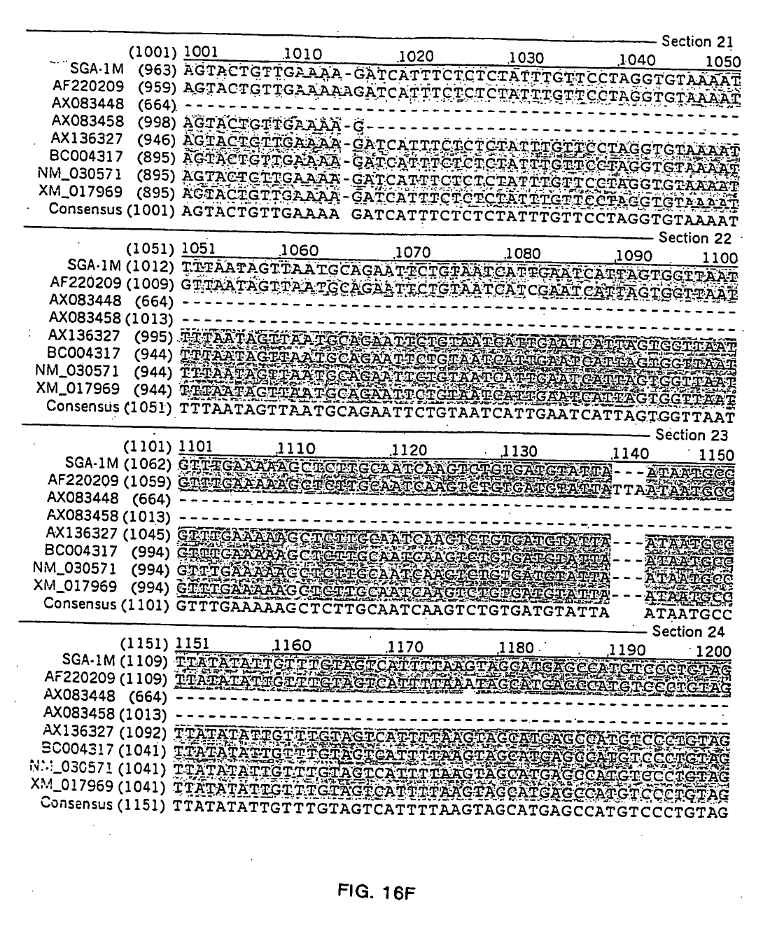

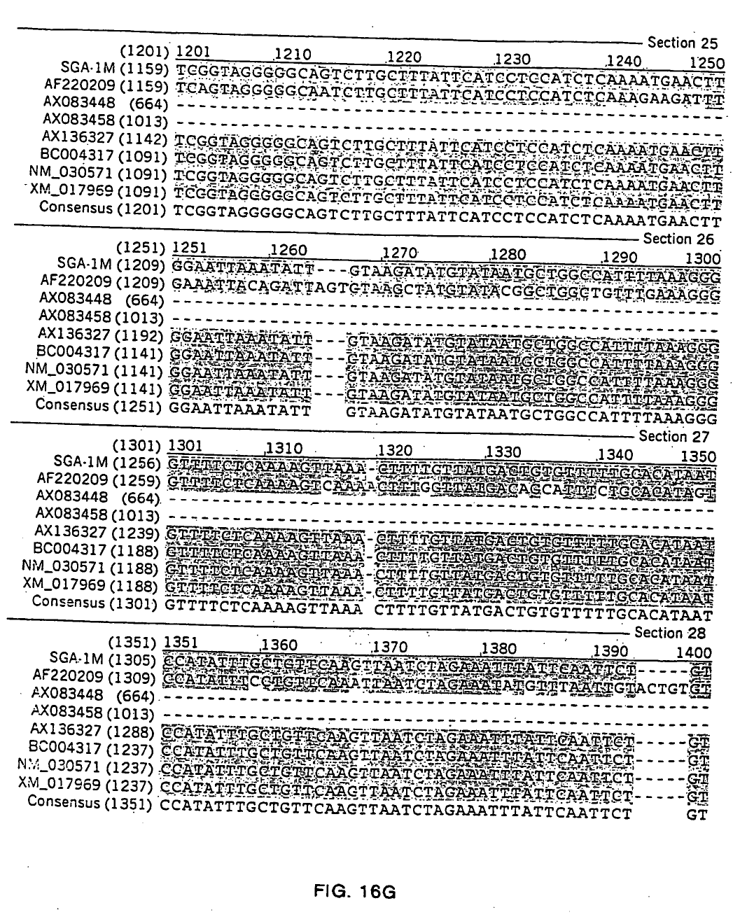

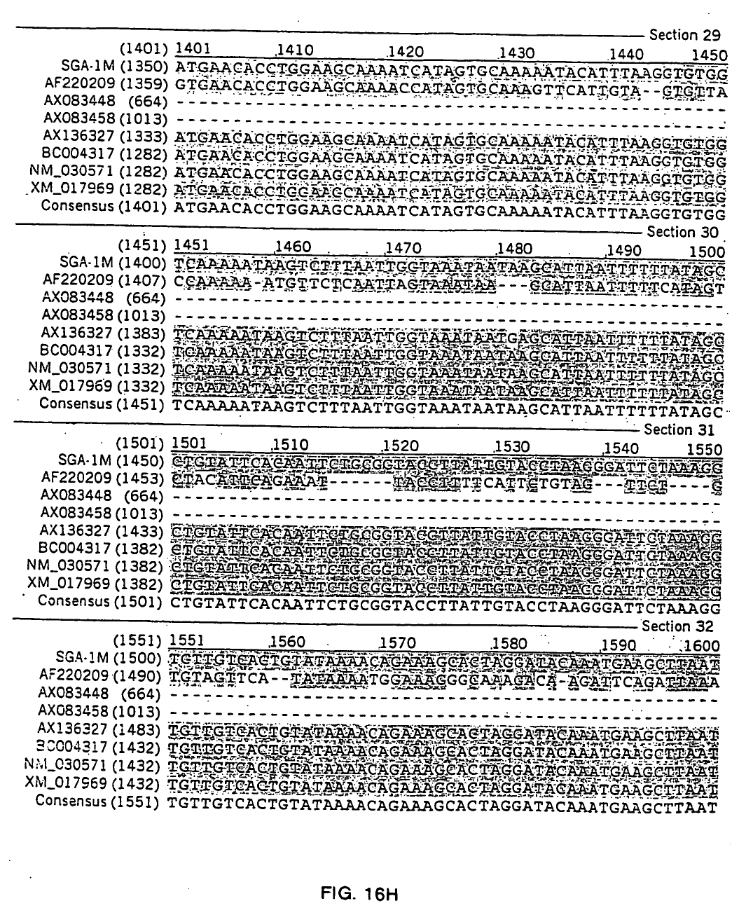

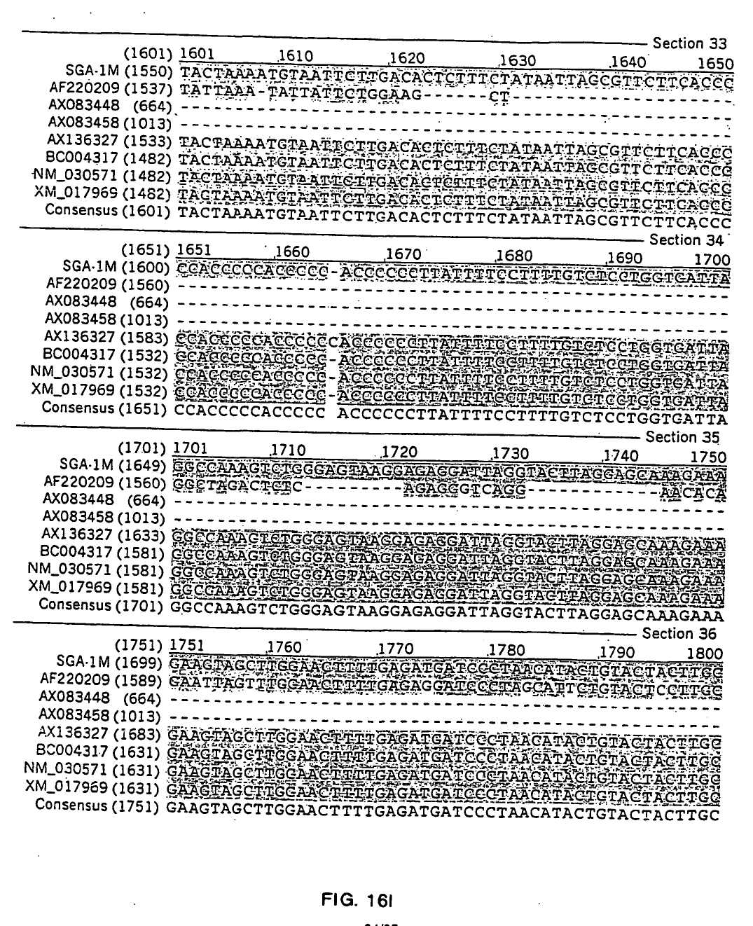

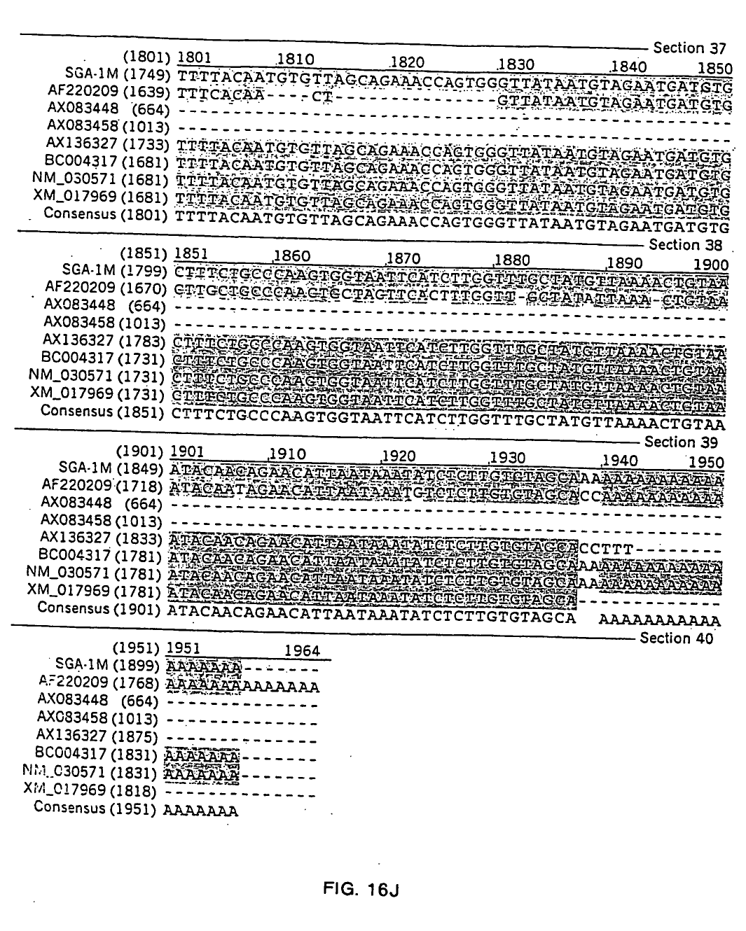

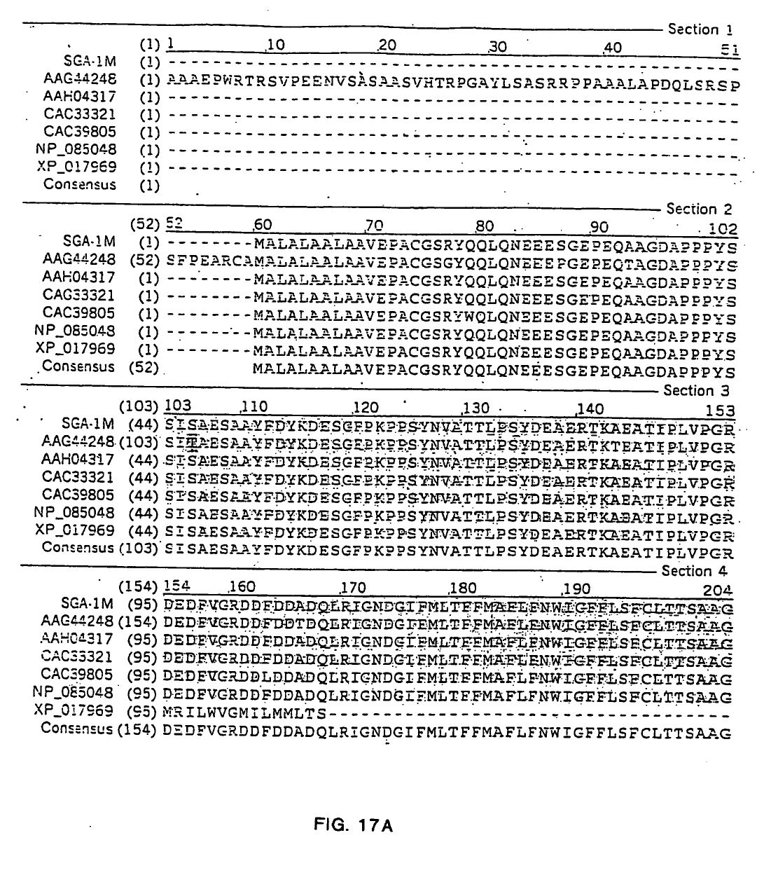

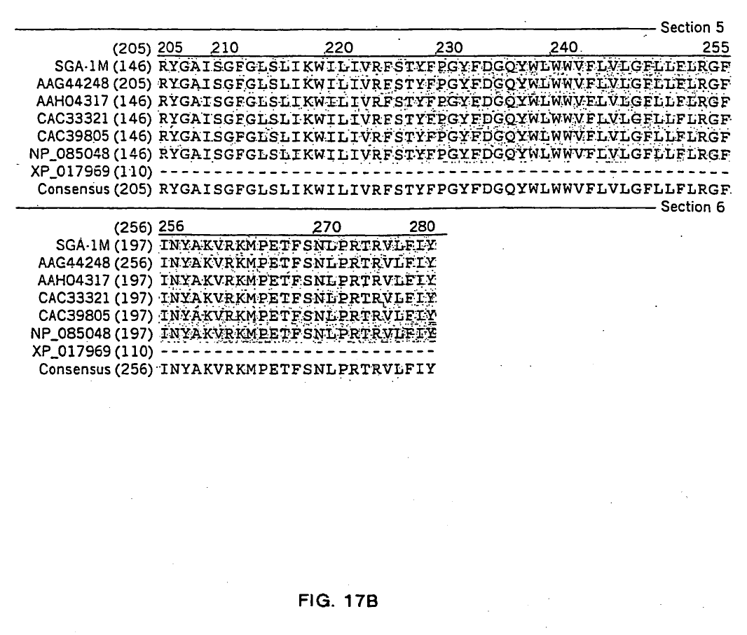

SGA-1M full-length cDNA was analyzed using Vector NTI Suite 6.0 (InforMax, Inc., Bethesda, Md.). A search of GenBank revealed several recent nucleic acid (FIG. 16) with varying degrees of similarity to SGA-1M (SEQ ID NO:1), as well as amino acid (FIG. 17) entries similar to SGA-1M(A) (SEQ ID NO:3) (See European Patent Application No. EP 1 067 182 A2, GenBank #AX136327 and PCT Publication No. WO 01/12660, GenBank #AX083448, AX083458, and PCT Publication No. WO 02/06312). SGA-1M remains uncharacterized in the context of a cancer-selective target prior to the present disclosure.

GenBank entry AF220209 corresponds to the mouse homolog for the Nedd4 binding protein and is 80% similar to SGA-1M(A) (SEQ ID NO:3; FIG. 17). Nedd4 is a ubiquitin-ligase which facilitates turnover of membrane-associated proteins (Jolliffe et al., 2000, J. Biochem. 351:557). The numberofidentified proteins, and specifically membraneproteins, associatedwith the regulation of ubiquitin-stimulated endocytosis is increasing (Hicke et aL, 1999, Trends Cell Biol. 9:107). The WW domains of Nedd-4 family members are proposed to interact with PY motifs of their binding partners (Jolliffe et aL, supra). SGA-1M(A) contains three such PY motifs, PPPY at 39-42aa, PPSY at 64-67aa, and PSY at 74-76aa (FIG. 17).

More recently, Nedd4-like proteins have been shown to interact with the latent membrane protein 2A (LMP2A) of Epstein-Barr Virus (EBV) and it is not yet clear whether Nedd4 regulates its turnover or whether this interaction disables the normal regulatory functions of Nedd4 (Ikeda et al., 2000, Virology 268:178). Studies focusing on the role of inappropriately expressed Nedd4 family members, and their ability to mediate protein turnover through ubiquitin-dependent regulation, can provide insight into a variety of different disease states.

There is no reported homology to the coding sequence (CDS) detailed as SGA-1M(B)(SEQ ID NO:5) (FIG. 2B and FIG. 10B) within GenBank. Homology to the nucleic acid region corresponding to SGA-1M(B), spanning 1104-1328 bp of the SGA-1M cDNA (SEQ ID NO:4; FIG. 2), is detailed in GenBank #AX136327 (European Patent Application No. EP 1 067 182 A2). No reference to the protein corresponding to SGA-1M(B) is provided. Thus, the 75 amino acid protein corresponding to SGA-1M(B) has not been disclosed within GenBank prior to the present disclosure.

The non-overlapping ORF's corresponding to SGA-1M(A)(SEQ ID NO:2) and SGA-1M(B)(SEQ ID NO:4) are similar to examples of dicistronic mRNA's (Kozak et al., 2001, Molecular and Cellular Biology 21(6):1899-1907). Alternative methods for initiation of internal translation within long mRNA transcripts are detailed in the literature (Pestova et al., 2001, Proc. Natl. Acad. Sci. USA 98(13):7029-7036; Vagner et al., 2001, EMBO Reports 2(10):893-898).

The predicted amino acid sequence corresponding to SGA-1M(B) (SEQ ID NO:5, FIG. 10B) includes a signal peptide sequence from (1-20) amino acids using the Signal IP program (Nielson et al., 1997, Protein Engineering 10:1-6). In addition, the amino acid sequence for SGA-1M(B) (FIG. 10B) contains two CXXC motifs. The CXXC motif is very similar to the various chemokine receptor classifications (Zlotnik et al., 2000, Immunity, 12(2):121-127). Chemokines are cited in the literature in association with progression to cancer metastasis (Murphy et al., 2001, N. Engl. J. Med. 345(11):833-835; Moore et al., 2001, Bioessays 23(8):674-676).

The active site CXXC motifs within oxidoreductases are essential for catalyzing redox reactions (Chivers et al., 1997, Biochemistry 36(14):4061-4066). Thioredoxin (Trx) is an oxidoreductase containing a CXXC active site that can be released by various cell types upon activation (Bertini et al., 1999, Journal of Experimental Medicine 189(11): 1783-1789). The chemotactic action of Thioredoxin differs from that of known chemokine receptors in that it is G-coupled independent. The hypothesis that a chemo-attractant acts via its enzymatic activity, instead of via classical receptor binding, allows for the possibility of using enzymatic inhibitors to illicit function instead of receptor antagonists or antibodies (Bertini et al., 1999, Journal of Experimental Medicine, 189(11): 1783-1789). The variations within CXXC motifs alter the overall capacity to assist in electron flow and thus influence the role of oxidoredectases in varying disease states. In addition, the forniation of native disulfide bonds in cellular proteins is often catalyzed in vitro by protein disulfide isomerase (PDI), which contains the active site CXXC motif (Chivers et al., 1996, EMBO J. 15(11):2659-2667). The formation of native disulfide bonds is necessary for maintaining efficient cellular response mechanisms. Therapeutic targeting of novel chemokines can assist in the prevention, diagnosis, prognosis, and overall treatment of cancer.

### 6.4. Discussion

Gene expression profiling provides a systematic approach to studying the mechanisms associated with progression from normal to metastatic disease. In this application, we have combined SSH and cDNA microarrays to identify the uncharacterized breast cancer-associated antigen, SGA-1M. Combining SSH and cDNA microarrays provides a rapid and effective approach to high-throughput screening for novel tumor targets. The principle of SSH allows for the preferential amplification of differentially expressed sequences while suppressing those present at equal abundance within the initial mRNA (Diatchenko et al., supra). The high level of enrichment, low level of background, and efficient normalization of sequences makes this an attractive approach for the rapid identification of novel targets. SGA-1M cDNA, identified by this method, provides a new target for breast cancer therapy, as well as a breast cancer marker for diagnosis and prognosis. SGA-1M is over-expressed in breast cancer, and other cancers, while displaying minimal expression in normal tissues. SGA-1M displayed tumor over-expression in 44% of the breast cancer patients analyzed by expression array. SGA-1M exhibited breast cancer-selective expression by differential array screening, RT-PCR, northern, FACS, and IHC staining. SGA-1M also stained positive by IHC for the following tumor types: breast adenocarcinoma, melanoma, thyroid cancer, lymphoma, pancreatic adenocarcinoma, and stomach adenocarcinoma. SGA-1M, based on its tumor-selective expression is an appropriate target for therapeutic intervention in breast cancer and other cancers.

### 7. REFERENCES CITED

All references cited herein are incorporated herein by reference in their entirety and for all purposes to the same extent as if each individual publication or patent or patent application was specifically and individually indicated to be incorporated by reference in its entirety for all purposes.

Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

What is claimed is:

1. A method of diagnosing cancer in a subject comprising detecting or measuring an SGA-1M gene product in a sample derived from said subject, wherein said SGA-1M gene product is:
a. an RNA corresponding to SEQ ID NO:1, or a nucleic acid derived therefrom;
b. a protein comprising SEQ ID NO:3;
c. a protein comprising SEQ ID NO:5;
d. a nucleic acid comprising a sequence hybridizable to SEQ ID NO:1 or its complement under conditions of high stringency, or a protein comprising a sequence encoded by said hybridizable sequence;
e. a nucleic acid comprising a sequence hybridizable to SEQ ID NO:2 or its complement under conditions of high stringency, or a protein comprising a sequence encoded by said hybridizable sequence;
f. a nucleic acid comprising a sequence hybridizable to SEQ ID NO:4 or its complement under conditions of high stringency, or a protein comprising a sequence encoded by said hybridizable sequence;
g. a nucleic acid at least 90% homologous to SEQ ID NO:1 or its complement as determined using the NBLAST algorithm;
h. a nucleic acid at least 90% homologous to SEQ ID NO:2 or its complement as determined using the NBLAST algorithm, or a protein encoded thereby; or
i. a nucleic acid at least 90% homologous to SEQ ID NO:4 or its complement as determined using the NBLAST algorithm, or a protein encoded thereby;
in which elevated levels of the SGA-1M gene product compared to a non-cancerous sample or a pre-determined standard value for a noncancerous sample, indicates the presence of cancer in the subject.

2. The method of claim 1 wherein the subject is a human.

3. The method of claim 1 or 2 wherein the cancer is breast cancer, ovarian cancer, skin cancer, a cancer of the lymphoid system, thyroid cancer, pancreatic cancer, stomach cancer, or lung cancer.

4. The method of claim 1 or 2 in which the sample is a tissue sample.

5. The method of claim 1 or 2 in which the sample is a plurality of cells.

6. The method of claim 1 or 2 in which the sample is a bodily fluid.

7. The method of claim 1 or 2 wherein said SGA-1M gene product is said protein comprising SEQ ID NO:3.

8. The method of claim 1 or 2 wherein said SGA-1M gene product is said protein comprising SEQ ID NO:5.

9. The method of claim 1 or 2 wherein said SGA-1M gene product is an mRNA corresponding to SEQ ID NO:1.

10. The method of claim 9 wherein said SGA-1M gene product is an mRNA corresponding to SEQ ID NO:2.

11. The method of claim 9 wherein said SGA-1M gene product is an mRNA corresponding to SEQ ID NO:4.

12. The method of claim 1 or 2 wherein an antibody that is specific to the SGA-1M gene product is used for detecting or measuring the SGA-1M gene product.

13. The method of claim 12, wherein the antibody immunospecifically binds to SEQ ID NO:3.

14. The method of claim 12, wherein the antibody immunospecifically binds to SEQ ID NO:5.

15. The method of claim 1 or 2 wherein an oligonucleotide that is specific to the SGA-1M gene product is used for detecting or measuring the SGA-1M gene product.

16. The method of claim 15, wherein the oligonucleotide is a DNA oligonucleotide.

17. A method of staging cancer in a subject comprising detecting or measuring an SGA-1M gene product in a sample derived from said subject, wherein said SGA-1M gene product is:
a. an RNA corresponding to SEQ ID NO: 1, or a nucleic acid derived therefrom;
b. a protein comprising SEQ ID NO:3;
c. a protein comprising SEQ ID NO:5;
d. a nucleic acid comprising a sequence hybridizable to SEQ ID NO:1 or its complement under conditions of high stringency, or a protein comprising a sequence encoded by said hybridizable sequence;
e. a nucleic acid comprising a sequence hybridizable to SEQ ID NO:2 or its complement under conditions of high stringency, or a protein comprising a sequence encoded by said hybridizable sequence;
f. a nucleic acid comprising a sequence hybridizable to SEQ ID NO:4 or its complement under conditions of high stringency, or a protein comprising a sequence encoded by said hybridizable sequence;
g. a nucleic acid at least 90% homologous to SEQ ID NO:1 or its complement as determined using the NBLAST algorithm;
h. a nucleic acid at least 90% homologous to SEQ ID NO:2 or its complement as determined using the NBLAST algorithm, or a protein encoded thereby; or
i. a nucleic acid at least 90% homologous to SEQ ID NO:4 or its complement as determined using the NBLAST algorithm, or a protein encoded thereby;
in which elevated levels of the SGA-1M gene product compared to a non-cancerous sample or a predetermined standard value for a noncancerous sample, indicates an advanced stage of cancer in the subject.

18. The method of claim 17 wherein the subject is a human.

19. The method of claim 17 or 18 wherein the cancer is breast cancer, ovarian cancer, skin cancer, a cancer of the lymphoid system, thyroid cancer, pancreatic cancer, stomach cancer, or lung cancer.

20. The method of claim 17 or 18 wherein the cancer involves regional lymph nodes.

21. The method of claim 17 or 18 wherein the cancer involves distant metastases.

22. The method of claim 17 or 18 in which the sample is a tissue sample.

23. The method of claim 17 or 18 in which the sample is a plurality of cells.

24. The method of claim 17 or 18 in which the sample is a bodily fluid.

25. The method of claim 17 or 18 wherein said SGA-1M gene product is said protein comprising SEQ ID NO:3.

26. The method of claim 17 or 18 wherein said SGA-1M gene product is said protein comprising SEQ ID NO:5.

27. The method of claim 17 or 18 wherein said SGA-1M gene product is an mRNA corresponding to SEQ ID NO:1.

28. The method of claim 27 wherein said SGA-1M gene product is an mRNA corresponding to SEQ ID NO:2.

29. The method of claim 27 wherein said SGA-1M gene product is an mRNA corresponding to SEQ ID NO:4.

30. The method of claim 17 or 18 wherein an antibody that is specific to the SGA-1M gene product is used for detecting or measuring the SGA-1M gene product.

31. The method of claim 30, wherein the antibody immunospecifically binds a protein consisting essentially of the amino acid sequence of SEQ ID NO:3.

32. The method of claim 30, wherein the antibody immunospecifically binds a protein consisting essentially of the amino acid sequence of SEQ ID NO:5.

33. The method of claim 17 or 18 wherein an oligonucleotide that is specific to the SGA-1M gene product is used for detecting or measuring the SGA-1M gene product.

34. The method of claim 33, wherein the oligonucleotide is a DNA oligonucleotide.

35. A method of vaccinating a subject against cancer comprising administering to the subject a molecule that elicits an immune response to an SGA-1M gene product, wherein said SGA-1M gene product is:
a. an RNA corresponding to SEQ ID NO:1, or a nucleic acid derived therefrom;
b. a protein comprising SEQ ID NO:3;
c. a protein comprising SEQ ID NO:5;
d. a nucleic acid comprising a sequence hybridizable to SEQ ID NO:1 or its complement under conditions of high stringency, or a protein comprising a sequence encoded by said hybridizable sequence;
e. a nucleic acid comprising a sequence hybridizable to SEQ ID NO:2 or its complement under conditions of high stringency, or a protein comprising a sequence encoded by said hybridizable sequence;
f. a nucleic acid comprising a sequence hybridizable to SEQ ID NO:4 or its complement under conditions of high stringency, or a protein comprising a sequence encoded by said hybridizable sequence;
g. a nucleic acid at least 90% homologous to SEQ ID NO:1 or its complement as determined using the NBLAST algorithm;
h. a nucleic acid at least 90% homologous to SEQ ID NO:2 or its complement as determined using the NBLAST algorithm, or a protein encoded thereby; or
i. a nucleic acid at least 90% homologous to SEQ ID NO:4 or its complement as determined using the NBLAST algorithm, or a protein encoded thereby;
j. a DNA molecule comprising SEQ ID NO:1;
k. a DNA molecule comprising SEQ ID NO:2; or
l. a DNA molecule comprising SEQ ID NO:4.

36. The method of claim 35 wherein said subject is a human.

37. The method of claim 35 wherein the molecule is an isolated DNA molecule comprising SEQ ID NO:1.

38. The method of claim 35 wherein the molecule is an isolated DNA molecule comprising SEQ ID NO:2.

39. The method of claim 35 wherein the molecule is an isolated DNA molecule comprising SEQ ID NO:4.

40. The method of claim 35 wherein the molecule is an isolated protein comprising SEQ ID NO:3.

41. The method of claim 35 wherein the molecule is an isolated protein comprising SEQ ID NO:5.

42. The method of claim 35, 36, 37, 38, 39, 40, or 41, wherein the cancer is breast cancer, ovarian cancer, skin cancer, a cancer of the lymphoid system, thyroid cancer, pancreatic cancer, stomach cancer, or lung cancer.

43. The method of claim 35, 36, 37, 38, 39, 40, or 41 wherein the immune response is a cellular immune response.

44. The method of claim 35, 36, 37, 38, 39, 40, or 41 wherein the immune response is a humoral immune response.

45. The method of claim 35, 36, 37, 38, 39, 40, or 41 wherein the immune response is both a cellular and a humoral immune response.

46. A method of determining if a subject is at risk of developing cancer, said method comprising:
a. measuring an amount of an SGA-1M gene product in a sample derived from the subject, wherein said SGA-1M gene product is:
i. an RNA corresponding to SEQ ID NO:1, or a nucleic acid derived therefrom;
ii. a protein comprising SEQ ID NO:3;
iii. a protein comprising SEQ ID NO:5;
iv. a nucleic acid comprising a sequence hybridizable to SEQ ID NO:1 or its complement under conditions of high stringency, or a protein comprising a sequence encoded by said hybridizable sequence;
v. a nucleic acid comprising a sequence hybridizable to SEQ ID NO:2 or its complement under conditions of high stringency, or a protein comprising a sequence encoded by said hybridizable sequence;
vi. a nucleic acid comprising a sequence hybridizable to SEQ ID NO:4 or its complement under conditions of high stringency, or a protein comprising a sequence encoded by said hybridizable sequence;
vii. a nucleic acid at least 90% homologous to SEQ ID NO:1 or its complement as determined using the NBLAST algorithm;
viii. a nucleic acid at least 90% homologous to SEQ ID NO:2 or its complement as determined using the NBLAST algorithm, or a protein encoded thereby; or
ix. a nucleic acid at least 90% homologous to SEQ ID NO:4 or its complement as determined using the NBLAST algorithm, or a protein encoded thereby; and
b. comparing the amount of said SGA-1M gene product in the subject with the amount of SGA-1M gene product present in a non-cancerous sample or predetermined standard for a noncancerous sample, wherein an elevated amount of said SGA-1M gene product in the subject compared to the amount in the non-cancerous sample or predetermined standard for a noncancerous sample indicates a risk of developing cancer in the subject.

47. The method of claim 46 wherein said subject is a human.

48. The method of claim 46 wherein the cancer is breast cancer, ovarian cancer, skin cancer, a cancer of the lymphoid system, thyroid cancer, pancreatic cancer, stomach cancer, or lung cancer.

49. The method of claim 46 or 47 wherein said SGA-1M gene product is said protein comprising SEQ ID NO:3.

50. The method of claim 46 or 47 wherein said SGA-1M gene product is said protein comprising SEQ ID NO:5.

51. The method of claim 46 or 47 wherein said SGA-1M gene product is said mRNA corresponding to SEQ ID NO:1.

52. The method of claim 51, wherein said SGA-1M gene product is said mRNA corresponding to SEQ ID NO:2.

53. The method of claim 51, wherein said SGA-1M gene product is said mRNA corresponding to SEQ ID NO:4.

54. The method of claim 46 or 47 wherein an antibody that is specific to the SGA-1M gene product is used for measuring the amount of the SGA-1M gene product.

55. The method of claim 54, wherein the antibody immunospecifically binds to a protein consisting essentially of the amino acid sequence of SEQ ID NO:3.

56. The method of claim 54, wherein the antibody immunospecifically binds to a protein consisting essentially of the amino acid sequence of SEQ ID NO:5.

57. The method of claim 46 or 47 wherein an oligonucleotide that is specific to the SGA-1M gene product is used for measuring the amount of the SGA-1M gene product.

58. The method of claim 57, wherein the oligonucleotide is a DNA oligonucleotide.

59. A method of determining if a subject suffering from cancer is at risk of metastasis of said cancer, said method comprising measuring an amount of an SGA-1M gene product in a sample derived from the subject, wherein said gene product is:
a. an RNA corresponding to SEQ ID NO:1, or a nucleic acid derived therefrom;
b. a protein comprising SEQ ID NO:3;
c. a protein comprising SEQ ID NO:5;
d. a nucleic acid comprising a sequence hybridizable to SEQ ID NO:1 or its complement under conditions of high stringency, or a protein comprising a sequence encoded by said hybridizable sequence;
e. a nucleic acid comprising a sequence hybridizable to SEQ ID NO:2 or its complement under conditions of high stringency, or a protein comprising a sequence encoded by said hybridizable sequence;
f. a nucleic acid comprising a sequence hybridizable to SEQ ID NO:4 or its complement under conditions of high stringency, or a protein comprising a sequence encoded by said hybridizable sequence;
g. a nucleic acid at least 90% homologous to SEQ ID NO:1 or its complement as determined using the NBLAST algorithm;
h. a nucleic acid at least 90% homologous to SEQ ID NO:2 or its complement as determined using the NBLAST algorithm, or a protein encoded thereby; or
i. a nucleic acid at least 90% homologous to SEQ ID NO:4 or its complement as determined using the NBLAST algorithm, or a protein encoded thereby;
wherein an elevated amount of SGA-1M gene product in the subject compared to the amount in the non-cancerous sample, or in the sample from the subject with the non-metastasizing cancer, or the amount in the predetermined standard for a noncancerous or non-metastasizing sample, indicates a risk of developing metastasis of said cancer in the subject.

60. The method of claim 59 wherein said subject is a human.

61. The method of claim 59 or 60 wherein the cancer is breast cancer, ovarian cancer, skin cancer, a cancer of the lymphoid system, thyroid cancer, pancreatic cancer, stomach cancer, or lung cancer.

62. The method of claim 59 or 60 in which the sample is a tissue sample.

63. The method of claim 59 or 60 in which the sample is a plurality of cells.

64. The method of claim 59 or 60 in which the sample is a bodily fluid.

65. The method of claim 59 or 60 wherein said SGA-1M gene product is said protein comprising SEQ ID NO:2.

66. The method of claim 59 or 60 wherein said SGA-1M gene product is an mRNA corresponding to SEQ ID NO:1.

67. The method of claim 59 or 60 wherein an antibody that is specific to the SGA-1M gene product is used for detecting or measuring the SGA-1M gene product.

68. The method of claim 67, wherein the antibody immunospecifically binds to a protein consisting essentially of the amino acid sequence of SEQ ID NO:3.

69. The method of claim 67, wherein the antibody immunospecifically binds to a protein consisting essentially of the amino acid sequence of SEQ ID NO:5.

70. The method of claim 59 or 60 wherein an oligonucleotide that is specific to the SGA-1M gene product is used for detecting or measuring the SGA-1M gene product.

71. The method of claim 70, wherein the oligonucleotide is a DNA oligonucleotide.

72. A method of screening for a compound that binds with an SGA-1M molecule, said method comprising:
a. contacting the SGA-1M molecule with a candidate agent, wherein said SGA-1M molecule is:
i. an RNA corresponding to SEQ ID NO:1, or a nucleic acid derived therefrom;
ii. a protein comprising SEQ ID NO:3;
iii. a protein comprising SEQ ID NO:5;
iv. a nucleic acid comprising a sequence hybridizable to SEQ ID NO:1 or its complement under conditions of high stringency, or a protein comprising a sequence encoded by said hybridizable sequence;
v. a nucleic acid comprising a sequence hybridizable to SEQ ID NO:2 or its complement under conditions of high stringency, or a protein comprising a sequence encoded by said hybridizable sequence;
vi. a nucleic acid comprising a sequence hybridizable to SEQ ID NO:4 or its complement under conditions of high stringency, or a protein comprising a sequence encoded by said hybridizable sequence;
vii. a nucleic acid at least 90% homologous to SEQ ID NO:1 or its complement as determined using the NBLAST algorithm;
viii. a nucleic acid at least 90% homologous to SEQ ID NO:2 or its complement as determined using the NBLAST algorithm, or a protein encoded thereby; or
ix. a nucleic acid at least 90% homologous to SEQ ID NO:4 or its complement as determined using the NBLAST algorithm, or a protein encoded thereby and
b. determining whether or not the candidate agent binds the SGA-1M molecule.

73. The method of claim 72 wherein said SGA-1M molecule is said RNA molecule corresponding to SEQ ID NO:1.

74. The method of claim 72 wherein said SGA-1M molecule is a DNA molecule that is at least 90% homologous to SEQ ID NO:1 or its complement as determined using the NBLAST algorithm.

75. The method of claim 72 wherein said SGA-1M molecule is a protein comprising SEQ ID NO:2.

76. The method of claim 72 wherein the screening assay is performed in vitro.

77. The method of claim 72 or 76 wherein the SGA-1M molecule is anchored to a solid phase.

78. The method of claim 72 or 76 wherein the candidate agent is anchored to a solid phase.

79. The method of claim 72 or 76 wherein the screening assay is performed in the liquid phase.

80. The method of claim 72 wherein said SGA-1M molecule is expressed on the surface of a cell or in the cytosol of a cell in step (a).

81. The method of claim 80 wherein the cell is engineered to express the SGA-1M molecule.

82. The method of claim 81 wherein the SGA-1M molecule is said protein comprising SEQ ID NO:2.

83. The method of claim 82 wherein said protein is expressed on the surface of said cell.

84. The method of claim 82 wherein said protein is expressed in the cytosol of said cell.

85. The method of claim 72 wherein the candidate agent is labeled radioactively.

86. The method of claim 72 wherein the candidate agent is labeled enzymatically.

87. The method of claim 72 wherein said SGA-1M molecule is at least 80% homologous to SEQ ID NO:1 as determined using the NBLAST algorithm.

88. The method of claim 87 wherein the screening assay is performed in vitro.

89. The method of claim 87 or 88 wherein the SGA-1M molecule is anchored to a solid phase.

90. The method of claim 87 or 88 wherein the candidate agent is anchored to a solid phase.

91. The of claim 87 or 88 wherein the screening assay is performed in the liquid phase.

92. The method of claim 87 wherein said SGA-1M molecule is expressed on the surface of a cell or in the cytosol of a cell in step (a).

93. The method of claim 92 wherein the cell is engineered to express the SGA-1M molecule.

94. The method of claim 87, wherein the candidate agent is labeled radioactively.

95. The method of claim 87, wherein the candidate agent is labeled enzymatically.

96. A method of screening for an intracellular protein that interacts with an SGA-1M gene product, said method comprising
a. immunoprecipitating the SGA-1M gene product from a cell lysate,wherein said SGA-1M gene product is:
i. an RNA corresponding to SEQ ID NO:1, or a nucleic acid derived therefrom;
ii. a protein comprising SEQ ID NO:3;
iii. a protein comprising SEQ ID NO:5;
iv. a nucleic acid comprising a sequence hybridizable to SEQ ID NO:1 or its complement under conditions of high stringency, or a protein comprising a sequence encoded by said hybridizable sequence;
v. a nucleic acid comprising a sequence hybridizable to SEQ ID NO:2 or its complement under conditions of high stringency, or a protein comprising a sequence encoded by said hybridizable sequence;
vi. a nucleic acid comprising a sequence hybridizable to SEQ ID NO:4 or its complement under conditions of high stringency, or a protein comprising a sequence encoded by said hybridizable sequence;
vii. a nucleic acid at least 90% homologous to SEQ ID NO:1 or its complement as determined using the NBLAST algorithm;
viii. a nucleic acid at least 90% homologous to SEQ ID NO:2 or its complement as determined using the NBLAST algorithm, or a protein encoded thereby; or
ix. a nucleic acid at least 90% homologous to SEQ ID NO:4 or its complement as determined using the NBLAST algorithm, or a protein encoded thereby;
and
b. determining whether or not any intracellular proteins bind to or form a complex with the SGA-1M gene product in the immunoprecipitate.

97. A method of screening for a candidate agent that modulates expression level of an SGA-1M gene, said method comprising:
a. contacting said SGA-1M gene with a candidate agent, wherein said SGA-1M gene is a nucleic acid at least 80% homologous to SEQ ID NO:1 as determined using the NBLAST algorithm; and
b. measuring the level of expression of an SGA-1M gene product, said SGA-1M gene product selected from the group consisting of an mRNA corresponding to SEQ ID NO:1, a protein comprising SEQ ID NO:3, and a protein comprising SEQ ID NO:5,
wherein an increase or decrease in said level of expression relative to said level of expression in the absence of said candidate agent indicates that the candidate agent modulates expression of an SGA-1M gene.

98. The method of claim 97 wherein said SGA-1M gene product is an mRNA corresponding to SEQ ID NO:1.

99. The method of claim 97 wherein said SGA-1M gene product is aprotein comprising SEQ ID NO:3.

100. The method of claim 97 wherein said SGA-1M gene product is a protein comprising SEQ ID NO:5.

101. A method of screening for a compound that is a candidate cancer therapeutic agent, comprising:
a. contacting an SGA-1M polypeptide with a compound;
b. determining whether an SGA-1M activity is modulated by the compound;
wherein a compound that modulates an SGA-1M activity is a candidate cancer therapeutic agent,
thereby identifying a candidate cancer therapeutic agent.

102. The method of claim 101, wherein the SGA-1M polypeptide is an SGA-1M(A) polypeptide.

103. The method of claim 101, wherein the SGA-1M polypeptide is an SGA-1M(B) polypeptide.

104. The method of claim 101, wherein contacting the SGA-1M polypeptide with the compound comprising contacting a cell that expresses the SGA-1M polypeptide with the compound.

105. The method of claim 101, wherein the activity modulated is:
a. a subcellular localization of the SGA-1M polypeptide;
b. an interaction between the SGA-1M polypeptide and a binding partner;
c. a post-translational modification of the SGA-1M polypeptide;
d. an activity of a protein whose activity is regulated or modulated by the SGA-1M polypeptide.

106. The method of claim 105, wherein SGA-1M polypeptide is an SGA-1M(A) polypeptide and the post-translational modification is ubiquitination.

107. The method of claim 105, wherein SGA-1M polypeptide is an SGA-1M(A) polypeptide and the protein is a sodium channel.

108. The method of claim 105, wherein SGA-1M polypeptide is an SGA-1M(A) polypeptide and the binding partner is a Nedd-4 protein.

109. The method of claim 101, wherein the modulation is an increase in said SGA-1M activity.

110. The method of claim 101, wherein the modulation is an inhibition in said SGA-1M activity.

111. A vaccine formulation for the prevention of cancer comprising:
a. an immunogenic amount of an SGA-1M gene product, wherein said SGA-1M gene product is:
an RNA corresponding to SEQ ID NO:1, or a nucleic acid derived therefrom;
i. an RNA corresponding to SEQ ID NO:1, or a nucleic acid derived therefrom;
ii. a protein comprising SEQ ID NO:3;
iii. a protein comprising SEQ ID NO:5;
iv. a nucleic acid comprising a sequence hybridizable to SEQ ID NO:1 or its complement under conditions of high stringency, or a protein comprising a sequence encoded by said hybridizable sequence;
v. a nucleic acid comprising a sequence hybridizable to SEQ ID NO:2 or its complement under conditions of high stringency, or a protein comprising a sequence encoded by said hybridizable sequence;
vi. a nucleic acid comprising a sequence hybridizable to SEQ ID NO:4 or its complement under conditions of high stringency, or a protein comprising a sequence encoded by said hybridizable sequence;
vii. a nucleic acid at least 90% homologous to SEQ ID NO:1 or its complement as determined using the NBLAST algorithm;
viii. a nucleic acid at least 90% homologous to SEQ ID NO:2 or its complement as determined using the NBLAST algorithm, or a protein encoded thereby; or
ix. a nucleic acid at least 90% homologous to SEQ ID NO:4 or its complement as determined using the NBLAST algorithm, or a protein encoded thereby;
and
b. a pharmaceutically acceptable excipient.

112. The vaccine formulation of claim 111 wherein said SGA-1M gene product is said nucleic acid at least 90% homologous to SEQ ID NO:1 or its complement as determined using the NBLAST algorithm.

113. The vaccine formulation of claim 111 wherein said SGA-1M gene product is said protein comprising SEQ ID NO:2.

114. An immunogenic composition comprising:
a. a purified SGA-1M gene product in an amount effective at eliciting an immune response, wherein said gene product is:
i. an RNA corresponding to SEQ ID NO: 1, or a nucleic acid derived therefrom;
ii. a protein comprising SEQ ID NO:3;
iii. a protein comprising SEQ ID NO:5;
iv. a nucleic acid comprising a sequence hybridizable to SEQ ID NO:1 or its complement under conditions of high stringency, or a protein comprising a sequence encoded by said hybridizable sequence;
v. a nucleic acid comprising a sequence hybridizable to SEQ ID NO:2 or its complement under conditions of high stringency, or a protein comprising a sequence encoded by said hybridizable sequence;
vi. a nucleic acid comprising a sequence hybridizable to SEQ ID NO:4 or its complement under conditions of high stringency, or a protein comprising a sequence encoded by said hybridizable sequence;
vii. a nucleic acid at least 90% homologous to SEQ ID NO:1 or its complement as determined using the NBLAST algorithm;
viii. a nucleic acid at least 90% homologous to SEQ ID NO:2 or its complement as determined using the NBLAST algorithm, or a protein encoded thereby; or
ix. a nucleic acid at least 90% homologous to SEQ ID NO:4 or its complement as determined using the NBLAST algorithm, or a protein encoded thereby;
and
b. an excipient.

115. The immunogenic composition of claim 114 wherein said SGA-1M gene product is said nucleic acid at least 90% homologous as determined using the NBLAST algorithm.

116. The immunogenic composition of claim 114 wherein said SGA-1M gene product is a protein comprising SEQ ID NO 2.

117. A pharmaceutical composition comprising:
a. an antibody which specifically binds to a protein consisting essentially of SEQ ID NO:3; and
b. a pharmaceutically acceptable carrier.

118. A pharmaceutical composition comprising:
a. an antibody which specifically binds to a protein consisting essentially of SEQ ID NO:5; and
b. a pharmaceutically acceptable carrier.

119. The pharmaceutical composition of claim 117 or 118 wherein said composition is formulated for delivery as an aerosol.

120. The pharmaceutical composition of claim 117 or 118 wherein said composition is formulated for-delivery parenterally.

121. The pharmaceutical composition of claim 117 or 118 wherein said composition is formulated for delivery orally.

122. A pharmaceutical composition comprising:
a. an SGA-1M gene product, wherein said gene product is: an RNA corresponding to SEQ ID NO:1, or a nucleic acid derived therefrom;
i. an RNA corresponding to SEQ ID NO:1, or a nucleic acid derived therefrom;
ii. a protein comprising SEQ ID NO:3;
iii. a protein comprising SEQ ID NO:5;
iv. a nucleic acid comprising a sequence hybridizable to SEQ ID NO:1 or its complement under conditions of high stringency, or a protein comprising a sequence encoded by said hybridizable sequence;
v. a nucleic acid comprising a sequence hybridizable to SEQ ID NO:2 or its complement under conditions of high stringency, or a protein comprising a sequence encoded by said hybridizable sequence;
vi. a nucleic acid comprising a sequence hybridizable to SEQ ID NO:4 or its complement under conditions of high stringency, or a protein comprising a sequence encoded by said hybridizable sequence;
vii. a nucleic acid at least 90% homologous to SEQ ID NO:1 or its complement as determined using the NBLAST algorithm;
viii. a nucleic acid at least 90% homologous to SEQ ID NO:2 or its complement as determined using the NBLAST algorithm, or a protein encoded thereby; or
ix. a nucleic acid at least 90% homologous to SEQ ID NO:4 or its complement as determined using the NBLAST algorithm, or a protein encoded thereby;
and
b. a pharmaceutically acceptable carrier,

123. The pharmaceutical composition of claim 122 wherein said SGA-1M gene product is said MRNA corresponding to SEQ ID NO:1.

124. The pharmaceutical composition of claim 123 wherein said SGA-1M gene product is said mRNA corresponding to SEQ ID NO:2.

125. The pharmaceutical composition of claim 123 wherein said SGA-1M gene product is said MRNA corresponding to SEQ ID NO:4.

126. The pharmaceutical composition of claim 122 wherein said SGA-1M gene product is said protein comprising SEQ ID NO:3.

127. The pharmaceutical composition of claim 122 wherein said SGA-1M gene product is said protein comprising SEQ ID NO:5.

128. The pharmaceutical composition of claim 122, 123, 126, 126, or 127, wherein said gene product is purified.

129. The pharmaceutical composition of claim 122, 123, 126, 126, or 127, wherein said composition is formulated for delivery as an aerosol.

130. The pharmaceutical composition of claim 122, 123, 126, 126, or 127, wherein said composition is formulated for delivery parenterally.

131. The pharmaceutical composition of claim 122, 123, 126, 126, or 127, wherein said composition is formulated for delivery orally.

132. A pharmaceutical composition comprising:
a. a purified nucleic acid comprising SEQ ID NO:2 or SEQ ID NO:4; and
b. a pharmaceutically acceptable carrier.

133. The pharmaceutical composition of claim 132 wherein said composition is formulated for delivery as an aerosol.

134. The pharmaceutical composition of claim 132 wherein said composition is formulated for delivery parenterally.

135. The pharmaceutical composition of claim 132 wherein said composition is formulated for delivery orally.

136. A method of diagnosing cancer in a subject comprising:
a. administering to said subject a compound that specifically binds a protein consisting essentially of the amino acid sequence of SEQ ID NO:3, wherein said compound is bound to an imaging agent; and
b. obtaining an internal image of said subject by use of said imaging agent;
wherein the localization or amount of said image indicates whether or not cancer is present in said subject.

137. A method of diagnosing cancer in a subject comprising:
a. administering to said subject a compound that specifically binds a protein consisting essentially of the amino acid sequence of SEQ ID NO:5, wherein said compound is bound to an imaging agent; and
b. obtaining an internal image of said subject by use of said imaging agent;
wherein the localization or amount of said image indicates whether or not cancer is present in said subject.

138. The method of claim 136 or 137 wherein said compound is an antibody.

139. The method of claim 138 wherein said antibody is conjugated to a radioactive metal and said obtaining step comprises recording a scintographic image obtained from the decay of the radioactive metal.

140. A kit comprising:
a. in one or more containers, a pair of oligonucleotide primers, each primer comprising an at least 5 nucleotide sequence complementary to a different strand of a double-stranded nucleic acid comprising SEQ ID NO: 1; and
b. in a separate container, a purified double-stranded nucleic acid comprising SEQ ID NO:1.

141. A method of testing the effects of a candidate therapeutic compound comprising administering said compound to the transgenic non-human animal of any one of claims ?-?; and determining any effects of said compound upon said transgenic non-human animal.

142. An isolated polypeptide comprising at least 8 amino acids of SEQ ID NO:5.

143. The polypeptide of claim 8, which comprises at least 10 amino acids of SEQ ID NO:5.

144. The polypeptide of claim 8, which comprises at least 15 amino acids of SEQ ID NO:5.

145. The polypeptide of claim 8, which comprises at least 20 amino acids of SEQ ID NO:5.

146. The polypeptide of claim 8, which comprises at least 50 amino acids of SEQ ID NO:5.

147. The polypeptide of any of claims 142-146 which is purified.

148. An isolated polypeptide which is encoded by a nucleic acid molecule comprising a nucleotide sequence which is at least 90% identical to a nucleic acid consisting of the nucleotide sequence of any of SEQ ID NO:4.

149. An antibody which selectively binds with the polypeptide of claim 142 or 148.

150. A host cell comprising a recombinant nucleic acid encoding the polypeptide of claim 142 operably linked to a promoter.

151. A host cell comprising a recombinant nucleic acid encoding the polypeptide of claim 148 operably linked to a promoter.

152. A method of producing the polypeptide of claim 142, comprising culturing the host cell of claim 150 under conditions in which the nucleic acid molecule is expressed.

153. A method of producing the polypeptide of claim 148, comprising culturing the host cell of claim 151 under conditions in which the nucleic acid molecule is expressed.


SEQUENCE LISTING (29 sequences)

SEQ ID NO: 1 - DNA, 1905 bp, Homo sapiens
ggcacgagagcttacagcctgagaagagcgtctcgcccgggagcggcggcggccatcgagacccaaccccaaggcgcgtccccctcggcctcccagcgctcccaagccgcagcggccgcgccccttcagctagctcgctcgctcgctctgcttccctgctgccggctgcgccatggcgttggcgttggcggcgctggcggcggtcgagccggcctgcggcagccggtaccagcagttgcagaatgaagaagagtctggagaacctgaacaggctgcaggtgatgctcctccaccttacagcagcatttctgcagagagcgcagcatattttgactacaaggatgagtctgggtttccaaagcccccatcttacaatgtagctacaacactgcccagttatgatgaagcggagaggaccaaggctgaagctactatccctttggttcctgggagagatgaggattttgtgggtcgggatgattttgatgatgctgaccagctgaggataggaaatgatgggattttcatgttaacttttttcatggcattcctctttaactggattgggtttttcctgtctttttgcctgaccacttcagctgcaggaaggtatggggccatttcaggatttggtctctctctaattaaatggatcctgattgtcaggttttccacctatttccctggatattttgatggtcagtactggctctggtgggtgttccttgttttaggctttctcctgtttctcagaggatttatcaattatgcaaaagttcggaagatgccagaaactttctcaaatctccccaggaccagagttctctttatttattaaagatgttttctggcaaaggccttcctgcatttatgaattctctctcaagaagcaagagaacacctgcaggaagtgaatcaagatgcagaacacagaggaataatcacctgctttaaaaaaataaagtactgttgaaaagatcatttctctctatttgttcctaggtgtaaaattttaatagttaatgcagaattctgtaatcattgaatcattagtggttaatgtttgaaaaagctcttgcaatcaagtctgtgatgtattaataatgccttatatattgtttgtagtcattttaagtagcatgagccatgtccctgtagtcggtagggggcagtcttgctttattcatcctccatctcaaaatgaacttggaattaaatattgtaagatatgtataatgctggccattttaaaggggttttctcaaaagttaaacttttgttatgactgtgtttttgcacataatccatatttgctgttcaagttaatctagaaatttattcaattctgtatgaacacctggaagcaaaatcatagtgcaaaaatacatttaaggtgtggtcaaaaataagtctttaattggtaaataataagcattaattttttatagcctgtattcacaattctgcggtaccttattgtacctaagggattctaaaggtgttgtcactgtataaaacagaaagcactaggatacaaatgaagcttaattactaaaatgtaattcttgacactctttctataattagcgttcttcacccccacccccacccccaccccccttattttccttttgtctcctggtgattaggccaaagtctgggagtaaggagaggattaggtacttaggagcaaagaaagaagtagcttggaacttttgagatgatccctaacatactgtactacttgcttttacaatgtgttagcagaaaccagtgggttataatgtagaatgatgtgctttctgcccaagtggtaattcatcttggtttgctatgttaaaactgtaaatacaacagaacattaataaatatctcttgtgtagcaaaaaaaaaaaaaaaaaaaaa

SEQ ID NO: 2 - DNA, 663 bp, Homo sapiens
  CDS (1)..(663)
atggcgttggcgttggcggcgctggcggcggtcgagccggcctgcggcagccggtaccagcagttgcagaatgaagaagagtctggagaacctgaacaggctgcaggtgatgctcctccaccttacagcagcatttctgcagagagcgcagcatattttgactacaaggatgagtctgggtttccaaagcccccatcttacaatgtagctacaacactgcccagttatgatgaagcggagaggaccaaggctgaagctactatccctttggttcctgggagagatgaggattttgtgggtcgggatgattttgatgatgctgaccagctgaggataggaaatgatgggattttcatgttaacttttttcatggcattcctctttaactggattgggtttttcctgtctttttgcctgaccacttcagctgcaggaaggtatggggccatttcaggatttggtctctctctaattaaatggatcctgattgtcaggttttccacctatttccctggatattttgatggtcagtactggctctggtgggtgttccttgttttaggctttctcctgtttctcagaggatttatcaattatgcaaaagttcggaagatgccagaaactttctcaaatctccccaggaccagagttctctttatttat

SEQ ID NO: 3 - protein, 221 aa, Homo sapiens
MALALAALAAVEPACGSRYQQLQNEEESGEPEQAAGDAPPPYSSISAESAAYFDYKDESGFPKPPSYNVATTLPSYDEAERTKAEATIPLVPGRDEDFVGRDDFDDADQLRIGNDGIFMLTFFMAFLFNWIGFFLSFCLTTSAAGRYGAISGFGLSLIKWILIVRFSTYFPGYFDGQYWLWWVFLVLGFLLFLRGFINYAKVRKMPETFSNLPRTRVLFIY

SEQ ID NO: 4 - DNA, 225 bp, Homo sapiens
  CDS (1)..(225)
atgccttatatattgtttgtagtcattttaagtagcatgagccatgtccctgtagtcggtagggggcagtcttgctttattcatcctccatctcaaaatgaacttggaattaaatattgtaagatatgtataatgctggccattttaaaggggttttctcaaaagttaaacttttgttatgactgtgtttttgcacataatccatatttgctgttcaagttaatc

SEQ ID NO: 5 - protein, 75 aa, Homo sapiens [sequence omitted]

SEQ ID NO: 6 - DNA, 46 bp, Artificial
  Description of Artificial Sequence Primer
  modified_base (44)..(46): n= a, t, g, or c
  modified_base (45)..(45): n = i
ttttgtacaagctttttttttttttttttttttttttttttttnnn

SEQ ID NO: 7 - DNA, 44 bp, Artificial
  Description of Artificial Sequence Primer
ctaatacgactcactatagggctcgagcggccgcccgggcaggt

SEQ ID NO: 8 - DNA, 42 bp, Artificial
  Description of Artificial Sequence Primer
ctaatacgactcactatagggcagcgtggtcgcggccgaggt

SEQ ID NO: 9 - DNA, 22 bp, Artificial
  Description of Artificial Sequence Primer
ctaatacgactcactatagggc

SEQ ID NO: 10 - DNA, 22 bp, Artificial
  Description of Artificial Sequence Primer
tcgagcggccgcccgggcaggt

SEQ ID NO: 11 - DNA, 20 bp, Artificial
  Description of Artificial Sequence Primer
agcgtggtcgcggccgaggt

SEQ ID NO: 12 - DNA, 20 bp, Artificial
  Description of Artificial Sequence Primer
ctgttcctgttggccgagtc

SEQ ID NO: 13 - DNA, 21 bp, Artificial
  Description of Artificial Sequence Primer
cgatgcattgttatcattaac

SEQ ID NO: 14 - DNA, 20 bp, Artificial
  Description of Artificial Sequence Primer
caccctgagcagctcatcac

SEQ ID NO: 15 - DNA, 20 bp, Artificial
  Description of Artificial Sequence Primer
ggccagggtcacatttcacc

SEQ ID NO: 16 - DNA, 17 bp, Artificial
  Description of Artificial Sequence Primer
gtaaaacgacggccagt

SEQ ID NO: 17 - DNA, 18 bp, Artificial
  Description of Artificial Sequence Primer
caggaaacagctatgacc

SEQ ID NO: 18 - DNA, 20 bp, Artificial
  Description of Artificial Sequence Primer
taatacgactcactataggg

SEQ ID NO: 19 - DNA, 20 bp, Artificial
  Description of Artificial Sequence Primer
attaaccctcactaaaggga

SEQ ID NO: 20 - DNA, 24 bp, Artificial
  Description of Artificial Sequence Primer
ggctgcaggtgatgctcctccacc

SEQ ID NO: 21 - DNA, 27 bp, Artificial
  Description of Artificial Sequence Primer
atcatcccgacccacaaaatcctcatc

SEQ ID NO: 22 - DNA, 20 bp, Artificial
  Description of Artificial Sequence Primer
ctgttcctgttggccgagtc

SEQ ID NO: 23 - DNA, 21 bp, Artificial
  Description of Artificial Sequence Primer
cgatgcattgttatcattaac

SEQ ID NO: 24 - DNA, 42 bp, Artificial
  Description of Artificial Sequence Primer
gatcgaaagcttgccaccatggcgttggcgttggcggcgctg

SEQ ID NO: 25 - DNA, 39 bp, Artificial
  Description of Artificial Sequence Primer
gatcgagaattcataaataaagagaactctggtcctggg

SEQ ID NO: 26 - DNA, 42 bp, Artificial
  Description of Artificial Sequence Primer
gatcgagctagcgccaccatggcgttggcgttggcggcgctg

SEQ ID NO: 27 - DNA, 39 bp, Artificial
  Description of Artificial Sequence Primer
gatcgaaagcttataaataaagagaactctggtcctggg

SEQ ID NO: 28 - protein, 15 aa, Homo sapiens
KVRKMPETFSNLPRT

SEQ ID NO: 29 - protein, 12 aa, Homo sapiens
PGRDEDFVGRDD